(19) United States
(12) Patent Application Publication
(10) Pub. No.: US 2024/0173399 A1
(43) Pub. Date: May 30, 2024

(54) ADJUVANTED MUCOSAL SUBUNIT VACCINES FOR PREVENTING SARS-COV-2 TRANSMISSION AND INFECTION

(71) Applicant: The United States of America, as represented by the Secretary, Depart. of Health and Human Services, Bethesda, MD (US)

(72) Inventors: Jay A. Berzofsky, Bethesda, MD (US); Yongjun Sui, Potomac, MD (US)

(73) Assignee: The United States of America, as represented by the Secretary, Depart. of Health and Human Services, Bethesda, MD (US)

(21) Appl. No.: 18/275,925

(22) PCT Filed: Feb. 4, 2022
(86) PCT No.: PCT/US2022/015349

§ 371 Date: Aug. 4, 2023

Related U.S. Application Data
(60) Provisional application No. 63/146,279, filed on Feb. 5, 2021.

(51) Int. Cl. A61K 39/215; A61K 31/713; A61K 38/20; A61K 39/00; C12N 15/117

(52) U.S. Cl. CPC A61K 39/215; A61K 31/713; A61K 38/2086; C12N 15/117; A61K 2039/545; A61K 2039/55555; A61K 2039/55561; C12N 2770/20034

(57) ABSTRACT

Immunogenic compositions that include SARS-CoV-2 spike (S) protein, S1 protein, or S2 protein, and an adjuvant, such as alum, or a combination of CpG oligodeoxynucleotide, Poly I:C, and IL-15, and nanoparticle compositions that include SARS-CoV-2 S protein, S1 protein, or S2 protein, and CpG oligonucleotide, poly(I:C), and IL-15, are provided. Also provided are methods of using such compositions, for example a prime intramuscular administration followed by one or more intranasal boosters that include the disclosed nanoparticles, to generate an immune response to SARS-CoV-2 in a subject, for example respiratory mucosal immunity, for example to prevent SARS-CoV-2 infection or transmission to other subjects.

### CROSS-REFERENCE TO RELATED APPLICATION

This application claims priority to U.S. 63/146,279, filed Feb. 5, 2021, herein incorporated by reference in its entirety.

### ACKNOWLEDGMENT OF GOVERNMENT SUPPORT

This invention was made with government support under project number Z01 BC-011941 awarded by the National Institutes of Health. The government has certain rights in the invention.

### FIELD

This disclosure relates to immunogenic compositions that include a SARS-CoV-2 spike (S) protein, S1 protein, or S2 protein, and one or more adjuvants, which can be administered intramuscularly in a prime vaccination, and nanoparticles that include a SARS-CoV-2 S protein, S1 protein, or S2 protein and one or more adjuvants, which can be administered intranasally in one or more booster vaccinations, to elicit an immune response to SARS-CoV-2.

### BACKGROUND

Severe acute respiratory syndrome coronavirus-2 (SARS-CoV-2), the virus responsible for the COVID-19 pandemic, has caused an unprecedented public health crisis. The pandemic highlighted the need for effective vaccines to reduce the spread of virus. Multiple vaccine strategies including adenovirus-vectored, inactivated virus, DNA-, mRNA-based platforms, and recombinant viral subunits/protein are under study to develop safe and effective vaccines against viral transmission and COVID-19 disease.

Most strategies in clinical trials are focused on systemically administered vaccines. Even though two mRNA vaccines and several others show protection in phase III trials, the duration of protection, there is a need for future boosts, and the ability to induce respiratory mucosal immunity is unknown. Mucosal immunity is important for COVID-19 because the virus infects via the ACE 2 receptor primarily through the upper and lower respiratory tracts. There is thus an urgent need for an effective mucosal vaccine and related therapeutic agents that can be used as a more effective boost to complement immunity from existing vaccines.

### SUMMARY

Provided herein are immunogenic compositions, which include a SARS-CoV-2 Spike (S) protein, S1 protein, or S2 protein and an adjuvant, such as alum, CpG oligodeoxynucleotide, poly I:C (e.g., a mismatched double-stranded RNA with one strand being a polymer of inosinic acid, the other a polymer of cytidylic acid) and IL-15. In one example, the composition includes a SARS-CoV-2 S1 protein having at least 80%, least 85%, at least 90%, at least 95%, at least 96%, at least 97%, at least 98%, at least 99%, or 100% sequence identity to SEQ ID NO: 2, 3, 4, 5, 6, 12 or 15, and one or more adjuvants, such as alum, or a combination of CpG oligodeoxynucleotide, poly I:C and IL-15. In one example, the composition includes a SARS-CoV-2 S2 protein having at least 80%, least 85%, at least 90%, at least 95%, at least 96%, at least 97%, at least 98%, at least 99%, or 100% sequence identity to SEQ ID NO: 9, 13, or 16, and one or more adjuvants, such as alum, or a combination of CpG oligodeoxynucleotide, poly I:C and IL-15. In one example, the composition includes a SARS-CoV-2 S protein having at least 80%, least 85%, at least 90%, at least 95%, at least 96%, at least 97%, at least 98%, at least 99%, or 100% sequence identity to SEQ ID NO: 1, 11 or 14, amino acids 16-1208 of SEQ ID NO: 1, amino acids 16-1205 of SEQ ID NO: 11, or amino acids 16-1206 of SEQ ID NO: 14, and one or more adjuvants, such as alum, or a combination of CpG oligodeoxynucleotide, poly I:C and IL-15. In some examples, the one or more adjuvants include alum, such as aluminum phosphate gel or aluminum hydroxide gel. In some examples, the one or more adjuvants include CpG oligodeoxynucleotide, poly I:C, IL-15, or combinations thereof, such as all of CpG oligodeoxynucleotide, poly I:C and IL-15. Such immunogenic compositions can further include a pharmaceutically acceptable carrier, such as water or saline. Also provided are vials, syringes, or other containers containing such immunogenic compositions. In some examples, such compositions are liquid, freeze dried, or frozen.

Also provided herein are nanoparticles, which include a SARS-CoV-2 S protein, S1 protein, or S2 protein and an adjuvant, such as alum, CpG oligodeoxynucleotide, poly I:C and IL-15. In one example, the nanoparticles include a SARS-COV-2 S1 protein having at least 80%, least 85%, at least 90%, at least 95%, at least 96%, at least 97%, at least 98%, at least 99%, or 100% sequence identity to SEQ ID NO: 2, 3, 4, 5, 6, 12 or 15, and one or more adjuvants, such as alum, or a combination of CpG oligodeoxynucleotide, poly I:C and IL-15. In one example, the nanoparticles include a SARS-CoV-2 S2 protein having at least 80%, least 85%, at least 90%, at least 95%, at least 96%, at least 97%, at least 98%, at least 99%, or 100% sequence identity to SEQ ID NO: 9, 13, or 16, and one or more adjuvants, such as alum, or a combination of CpG oligodeoxynucleotide, poly I:C and IL-15. In one example, the nanoparticles include a SARS-CoV-2 S protein having at least 80%, least 85%, at least 90%, at least 95%, at least 96%, at least 97%, at least 98%, at least 99%, or 100% sequence identity to SEQ ID NO: 1, 11 or 14, amino acids 16-1208 of SEQ ID NO: 1, amino acids 16-1205 of SEQ ID NO: 11, or amino acids 16-1206 of SEQ ID NO: 14, and one or more adjuvants, such as alum, or a combination of CpG oligodeoxynucleotide, poly I:C and IL-15. In some examples, the one or more adjuvants comprise a TLR-9 agonist (e.g., CpG oligodeoxynucleotide), TLR-3 agonist (e.g., poly I:C), IL-15 (such as SEQ ID NO: 7), or combinations thereof. In some examples, the one or more adjuvants comprise TLR-9 agonist (e.g., CpG oligodeoxynucleotide), TLR-3 agonist (e.g., poly I:C), and IL-15 (such as SEQ ID NO: 7). In some examples, the nanoparticle comprises poly(d,1-lactide-co-glycolide) (PLGA). In some examples, the nanoparticle comprises 1,2-dioleoyl-sn-glycero-3-phosphoethanolamine (DOTAP). Such nanoparticles can be present in a composition that includes a pharmaceutically acceptable carrier, such as saline or water. Also provided are vials, syringes, or other containers containing such nanoparticles and nanoparticle-containing compositions. In some examples, such nanoparticle containing compositions are liquid, freeze dried, or frozen.

Methods of using the disclosed immunogenic compositions and nanoparticles to elicit an immune response against SARS-CoV-2 in a subject, such as a human or non-human mammal, are provided. In some examples, such methods include administering to the subject (for example intramuscularly) an effective amount of a primary dose of an immunogenic composition that includes a SARS-CoV-2 S1 protein comprising at least 80%, least 85%, at least 90%, at least 95%, at least 96%, at least 97%, at least 98%, at least 99%, or 100% sequence identity to SEQ ID NO: 2, 3, 4, 5, 6, 12 or 15, and one or more adjuvants, such as alum or a combination of CpG oligodeoxynucleotide, poly I:C and IL-15. In one example, such methods include administering to the subject (for example intramuscularly) an effective amount of a primary dose of an immunogenic composition that includes a SARS-CoV-2 S2 protein having at least 80%, least 85%, at least 90%, at least 95%, at least 96%, at least 97%, at least 98%, at least 99%, or 100% sequence identity to SEQ ID NO: 9, 13, or 16, and one or more adjuvants, such as alum, or a combination of CpG oligodeoxynucleotide, poly I:C and IL-15. In one example, such methods include administering to the subject (for example intramuscularly) an effective amount of a primary dose of an immunogenic composition that includes a SARS-CoV-2 S protein having at least 80%, least 85%, at least 90%, at least 95%, at least 96%, at least 97%, at least 98%, at least 99%, or 100% sequence identity to SEQ ID NO: 1, 11 or 14, amino acids 16-1208 of SEQ ID NO: 1, amino acids 16-1205 of SEQ ID NO: 11, or amino acids 16-1206 of SEQ ID NO: 14, and one or more adjuvants, such as alum, or a combination of CpG oligodeoxynucleotide, poly I:C and IL-15. In some examples, the primary dose instead includes an mRNA encoding a SARS-CoV-2 S protein, S1 protein, or S2 protein, such as one encoding a protein having at least 80%, least 85%, at least 90%, at least 95%, at least 96%, at least 97%, at least 98%, at least 99%, or 100% sequence identity to SEQ ID NO: 1, 2, 3, 4, 5, 6, 9, 11, 12, 13, 14, 15, 16, amino acids 16-1208 of SEQ ID NO: 1, amino acids 16-1205 of SEQ ID NO: 11, or amino acids 16-1206 of SEQ ID NO: 14, and one or more adjuvants (such as alum, or a combination of CpG oligodeoxynucleotide, poly I:C and IL-15). Subsequent to administering the primary dose, the method includes administering (such as intranasally) to the subject an effective amount of one or more booster doses of the disclosed nanoparticles that include a SARS-CoV-2 S protein, S1 protein, or S2 protein, such as one having at least 80%, least 85%, at least 90%, at least 95%, at least 96%, at least 97%, at least 98%, at least 99%, or 100% sequence identity to SEQ ID NO: 1, 2, 3, 4, 5, 6, 9, 11, 12, 13, 14, 15, 16, amino acids 16-1208 of SEQ ID NO: 1, amino acids 16-1205 of SEQ ID NO: 11, or amino acids 16-1206 of SEQ ID NO: 14, and one or more adjuvants that includes CpG oligodeoxynucleotide, poly I:C and/or IL-15, thereby eliciting the immune response. In some examples the nanoparticle includes PLGA. In some examples the nanoparticle includes DOTAP. In some examples, the primary dose is administered intramuscularly (IM), and the one or more booster doses are each administered intranasally (IN).

In some examples, a first booster dose is administered at least 2 weeks, at least 3 weeks, at least 4 weeks, at least 5 weeks, at least 6 weeks, at least 7 weeks, at least 8 weeks, at least 10 weeks, at least 12 weeks, at least 16 weeks, at least 6 months, at least 9 months, at least 12 months, at least 18 months, or at least 24 months after administering the primary dose. In some examples, the booster dose is at least one year after the primary dose. In some examples, the one or more booster doses comprises 2, 3, 4, 5, 6, 7, 8, 9 or 10 booster doses. In some examples, the immune response inhibits or prevents SARS-CoV-2 infection in the subject. In some examples, the immune response inhibits or prevents severe COVID19 disease in the subject. In some examples, the immune response inhibits replication of the SARS-CoV-2 in the subject. In some examples, the immune response increases production of dimeric IgA specific for SARS-CoV-2 S1 and IFNα. In some examples, the immune response provides sterilizing protection against SARS-CoV-2 to the subject, for example in the upper and lower respiratory tracts. In some examples, the immune response induces a neutralizing antibody titer of at least 100, at least 200, at least 300, at least 350, such as 374. In some examples, combinations of these effects are achieved.

In one example, the method of eliciting an immune response against SARS-CoV-2 in a subject, includes administering to the subject an effective amount of a primary dose an immunogenic composition including the SARS-CoV-2 S1 protein of SEQ ID NO: 2, and alum; and subsequently administering to the subject an effective amount of one or more booster doses of nanoparticles comprising the SARS-CoV-2 S1 protein of SEQ ID NO: 2, CpG oligodeoxynucleotide, poly I:C, and IL-15. In some examples, the first booster dose includes PLGA nanoparticles. In some examples, at least one booster dose administered after the first booster dose administered comprises DOTAP nanoparticles.

In some examples the methods further include administering to the subject a COVID-19 treatment, such as one or more of remdesivir, galidesivir, lenzilumab, molnupiravir, hydroxychloroquine, dexamethasone, arbidol, favipiravir, baricitinib, lopinavir/ritonavir, zinc ions, and interferon beta-1b.

The foregoing and other objects and features of the disclosure will become more apparent from the following detailed description, which proceeds with reference to the accompanying figures.

### BRIEF DESCRIPTION OF THE DRAWINGS

FIG. 1. Schematic diagram of immunization protocol and groups

FIGS. 2A-2E. Spike-specific humoral immune responses in PBMC and BAL samples of the vaccinated animals. The EC50 of S1-specific IgG in serum (A) and the area under the curve (AUC) of S1-specific IgG and IgA in BAL (B-C) were measured during the whole course of vaccination. Dimeric IgA responses in BAL at Day8 post last vaccination (D) and PRNT (neutralizing) titers against live virus (E) in the serum samples were measured. Mann-Whitney tests were used to compare the difference between groups in D. Short lines show medians. Dashed lines show the lower and upper assay limits.

FIG. 3. The kinetics of PRNT neutralizing titers (ID90) in the vaccinated groups. Short lines and dashed lines show medians, the lower and upper assay limits respectively.

FIG. 4. Spike-specific TNFα+ or IFNγ+CD4+ T cell responses in PBMC and BAL samples of the vaccinated animals

FIGS. 5A-5F. Spike-specific CD4+ T cell responses and trained immunity in PBMC and BAL samples of the vaccinated animals. Intracellular cytokine staining assays in responses to spike protein S1 were measured during the whole course of vaccination in PBMC (A) and BAL (B) samples. Spike-specific TNFα⁺CD4⁺ T cell responses of different groups in PBMC (C) and BAL (D) samples at week 2 post 2nd-, and Day 8 post last-vaccination were compared. The kinetics of CD14⁻/CD16+ (monocyte or possibly NK) subsets were measured in the BAL samples of the vaccinated animals after 18 hrs. of PMA+ ionomycin stimulation (E). IFN-α was measured in the supernatant of BAL samples after 18 hrs. of poly I:C+S1 stimulation (F). Medians are shown. Mann-Whitney tests were used to compare the differences between groups in C-F.

FIG. 6. Spike-specific Th subsets in PBMC and BAL samples of the vaccinated animals at Day8 post last vaccination. The dashed lines show the assay limit (bars are the medians). Medians are shown. Dashed lines are the threshold for positive responses.

FIG. 7. The kinetics of PMA-stimulated Th1, Th2 and Th17 subsets in PBMCs and Th1 subsets in BALs of the vaccinated animals. Means with SEMs are shown.

FIG. 8. The Tc1 immune responses stimulated by PMA in the BAL samples of the vaccinated animals. Medians are shown in the upper panels (group 1 on the left of each panel, group 2 on the right of each panel), means with SEMs are shown in the lower panels.

FIG. 9. Spike-specific TNFα+ and/or IFNγ+CD8+ T cell responses in PBMC and BAL samples of the vaccinated animals. Medians are shown in the upper panels, means with SEMs are shown in the lower panels.

FIGS. 10A-10B. Chemokine and cytokine production upon stimulation with poly I:C+S1 in the BAL samples from the vaccinated animals. BAL samples from 1-week post 2^{nd} vaccination were treated with poly I:C+S1 protein for 18 hrs. before supernatant was collected for measurement of a total of 13 chemokines and cytokines. Means with SEMs are shown.

FIGS. 11A-11D. Viral load in nasal swabs and BAL fluids after SARS-CoV-2 intranasal/intratracheal challenges. SARS-CoV-2 genomic RNA and subgenomic RNA were assessed in the nasal swabs (A and B) BAL fluid (C and D) collected at days 2 and 4 after viral challenges. AUC was calculated for each animal and plotted in the box and whisker plots, where the median, other quartiles, and minimum to maximum are shown. The assay lower limit (50 copies) is shown as dashed lines. In each panel, Mann-Whitney U tests corrected for multiple comparisons by the Hochberg method were used to compare the viral load AUC differences between vaccinated groups and the SARS-CoV-2 naive control group. n=6 for group 1, group 2, and naive group. Each animal has a unique symbol with different shape and color, which is consistent throughout (A-D).

FIGS. 12A-12D. Immune correlations after vaccination and viral challenges in group 2. The P (A) and R values (B) of the immune correlation matrix among antigen-specific humoral, cellular responses, innate immunity, and genomic RNA in BAL at day 2. The peripheral and BAL samples were collected at day 8 after last vaccination or early time points (noted). Prechallenge IgA titer in BAL (C) and IFN-α production in ex vivo-stimulated BAL cells (D) were correlated with day 2 postchallenge genomic RNA in BAL. Spearman's R and P values are shown. n=6 for group 2.

FIG. 13. Pathological Summary of the animals challenged with SARS-CoV-2 virus.

FIG. 14A-14C. Histopathological analysis and viral antigen detection in the lung. Seven or 10 days after challenge, lungs were harvested, and multiple sections of lung were evaluated histologically and immunohistochemically for the presence of SARS-CoV-2-related inflammation and SARS-CoV-2 virus antigen. Representative images from one animal in naïve group (A), and one animal in vaccinated group 1(B). Each animal was blindly scored by a pathologist based on the degree of inflammation in the lung. Mann-Whitney tests were used to compare the lung inflammation between naïve and vaccination groups (C). Scale bars represent 200 μm (4×) and 100 μm (10×). n=6 for groups 1 and 2 and naïve group.

FIG. 15. Schematic diagram of vaccination and viral challenge.

FIGS. 16A-16C. Humoral immune responses against SARS-CoV-2 spike protein 1 (S1) in vaccinated macaques. (A). Kinetics of S1-specific binding IgG titers in serum and BAL. Bars indicate geometric means of half-maximal binding titers and means of AUC. (B). PRNT titers in the serum samples of the vaccinated animals at 2-week after one-year boost. Geometric mean+geometric SD are shown. (C) S1-specific IgA and dimeric IgA responses in nasal swabs (NS) and BAL samples. Paired t-tests were used to compare the humoral responses after the booster. WA: WA1/2020 D614G SARS-CoV-2 strain; Wu: Wuhan original strain; Beta: B.1.351 variant. The dashed lines indicate the detection limits. Data are shown as mean+SEM. Blue color indicates the S1 protein or the virus from Wuhan or WA strain, and magenta color indicates from beta variant.

FIG. 17. T cell responses against SARS-COV-2 spike protein 1 (S1) in PBMC and BAL samples of the vaccinated macaques. The frequencies of IFNγ and/or TFNα-producing CD4⁺ and CD8⁺ T cells were stained and measured after stimulation with S1 either from Wuhan strain or from beta variant for 18 hrs in PBMC and BAL samples. The upper plots showed a representative cytokine gating of CD4+ T cells in medium-only control (left) and S1-stimulated (right) BAL sample from the same vaccinated animal. Dashed lines indicate the detection limits. Bars indicate medians. Blue color indicates the S1 protein from Wuhan strain, and magenta color indicates from beta variant.

FIG. 18A. T helper subsets in PBMC samples of the vaccinated macaques. The frequencies of IFNγ+, TFNα+, or IL4+CD4+ T cells were stained and measured after stimulation with PMA+ionomycin for 18 hrs in PBMCs from different time-points.

FIG. 18B. Inflammation and immunohistochemistry evaluation of the lung sections (necropsy at day 7 post SARS-CoV-2 challenge).

FIGS. 19A-19B. Viral burden in the nasal swabs (NS) and BAL samples after SARS-CoV-2 beta variant intranasal and intratracheal challenges. (A). TCID50 titer of the viral burdens in nasal swabs (NS) and BAL samples of individual animals (n=5 in the vaccine group and n=5 in the control group). (B). Area under curve (AUC) over time after challenge was calculated for each animal, representing total viral burdens. The total viral burdens were compared between vaccine and control groups in NS and BAL. Dashed lines indicate the detection limits. Box and whiskers with min to max were shown in the graph.

FIGS. 20A-20C. Histopathology in the lungs at day 7 post SARS-CoV-2 challenge. H & E and immunohistochemistry to detect SARS-COV-2 antigens were performed in the vaccinated (A) and naïve (B) animals. The upper rows of A-B were H & E staining, while the lower rows of a-b were immunohistochemistry of SARS-CoV-2 detection. All images 10× (scale bar=100 um). (C) Inflammation scores in the lung were compared between the vaccinated and naïve groups. Mann-Whitney test was used for comparison. Box and whiskers with min to max were shown in the graph.

FIGS. 21A-21C. Hamsters primed with S1 protein+alum and boosted with S1 protein+CP15 showed significant protection against weight loss after challenge with SARS-Cov-2 WA strain. (A) Schematic diagram of vaccination/challenge schedules, and vaccination groups. (B) Kinetics of body weight loss and (C) the area under curve of body weight loss in hamsters after challenged with SARS-CoV-2 WA strain. Two-way ANOVA and Mann-Whitney analysis were used to compare between the vaccinated groups and Naïve control group.

FIGS. 22A-22F. Hamsters IM primed and IN boosted with S1+CP15, showed significant oral swab viral load reduction after challenge with SARS-Cov-2 WA strain. Viral loads in oral swabs from (A) Group 1 primed and boosted with S1 protein adjuvanted with alum IM, (B) Group 2 primed with S1 protein adjuvanted with alum IM and then boosted with S1+CP15 IN, (C) Group 3 primed with S1+CP15 IM then boosted with S1+CP15 IN, (D) Group 3 administered PBS as a negative control. (E) Summary of the groups in (A)-(D). (F) Area under curve of oral swab viral loads from each group were shown. Two-way ANOVA and Mann-Whitney analysis were used to compare the vaccinated groups and control group.

FIGS. 23A-23I. Vaccine-induced neutralizing antibody titers did not correlate with protection after challenge with SARS-CoV-2 WA strain in Syrian golden hamsters. (A) PRNT titers against SARS-CoV-2 WA strain in the serum of vaccinated and naïve animals. (B-E) PRNT titers did not correlate with weight loss in the vaccinated animals. (F-I) PRNT titers did not correlate with viral load reduction in the oral swabs of the vaccinated animals. Spearman analyses were used for the correlations.

FIGS. 24A-24D. Gender difference on viral load reduction in oral swabs and weight loss after challenged with SARS-CoV-2 WA strain in Syrian golden hamsters. Kinetics of body weight loss and viral load reduction in vaccinated and naïve female (A) and male (B) hamsters after challenged with SARS-CoV-2 WA strain. Comparisons of area under curve of body weight loss (C) and viral load reduction (D) between female and male hamsters after SARS-CoV-2 WA strain infection. Two-way ANOVA analyses were used to compare the vaccinated groups and control group. Mann-Whitney test was used to compare the females and males.

FIGS. 25A-25C. Boosting with S1/IL15/CpG/poly:IC (S1+CP15) IN (group 2) provides significant protection against weight loss in hamsters. (A) Schematic diagram of vaccination/challenge schedules, and vaccination groups. (B) Kinetics of body weight loss and (C) the area under curve of body weight loss in hamsters after challenged with SARS-CoV-2 beta variant. Two-way ANOVA and Mann-Whitney analysis were used to compare between the vaccinated groups and Naïve control group.

FIGS. 26A-25D. Neither vaccine-induced nor virus-induced neutralizing antibody titers correlated with protection against weight loss after challenged with SARS-CoV-2 beta variant in Syrian golden hamsters. (A-B) PRNT titers against SARS-CoV-2 beta variant in the serum of the vaccinated and naïve animals (A) pre- and (B) post-beta variant challenge. (C-D) PRNT titers did not correlate weight loss (C) pre- and (D) post-beta variant challenge in the vaccinated and control animals. Spearman analyses were used for the correlations.

FIG. 27. Female mice boosted IN had more robust lung S1-specific IL-2+, IFNγ+, TNFα+CD4+ and CD8+ T cell responses. Dashed lines indicate the detection limits. Bars indicate medians.

### SEQUENCE LISTING

The nucleic and amino acid sequences provided herein are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing provided herewith, and generated on Feb. 4, 2022, is herein incorporated by reference.

SEQ ID NO: 1: Native SARS-CoV-2 S protein (including the signal peptide aa 1-15, native ectodomain and TM and CT domains) (NCBI Ref. No. YP_009724390.1, incorporated by reference herein). The S1 subunit sequence is underlined (and provided as SEQ ID NO: 2). The S2 subunit sequence is in bold (and provided as SEQ ID NO: 9).

MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTECSNLLLQYGSFC
TQLNRALTGIAVEQDKNTQEVFAQVKQIYKTPPIKDFGGF
NFSQILPDPSKPSKRSFIEDLLFNKVTLADAGFIKQYGDC
LGDIAARDLICAQKFNGLTVLPPLLTDEMIAQYTSALLAG
TITSGWTFGAGAALQIPFAMQMAYRFNGIGVTQNVLYENQ
KLIANQFNSAIGKIQDSLSSTASALGKLQDVVNQNAQALN
TLVKQLSSNFGAISSVLNDILSRLDKVEAEVQIDRLITGR
LQSLQTYVTQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHT
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYT

SEQ ID NO: 2 is a native SARS-CoV-2 S1 subunit protein sequence.

SEQ ID NO: 3 is a SARS-CoV-2 S1 subunit protein sequence from the B.1.1.7 variant, identified in the UK, which includes at least the following mutations in the S1 protein: N501Y substitution, 69-70del, and P681H substitution (numbering based on SEQ ID NO: 1).

SEQ ID NO: 4 is a SARS-CoV-2 S1 subunit protein sequence from the B.1.351 variant, identified in South Africa, which includes at least the following mutations in the S1 protein: L18F, D80A, D215G, R246I, K417N, E484K, N501Y, and D614G substitutions (numbering based on SEQ ID NO: 1, changes underlined). See for example Tegally et al, https://doi.org/10.1101/2020.12.21.20248640

SEQ ID NO: 5 is a native SARS-CoV-2 S1 subunit protein sequence from the P.1 variant, identified in Brazil, which includes at least the following mutations in the S1 protein: L18F, T20N, P26S, D138Y, R190S, K417T, E484K, N501Y, D614G and H655Y substitutions (numbering based on SEQ ID NO: 1, changes underlined). See for example Sabino et al., Lancet, https://doi.org/10.1016/S0140-6736(21)00183-5.

SEQ ID NO: 6 is a SARS-CoV-2 S1 subunit protein sequence, which includes the following mutations in the S1 protein E484K and N501Y substitutions (numbering based on SEQ ID NO: 1, changes underlined).

SEQ ID NO: 7 is a recombinant human IL-15 protein sequence, Asn49-Ser162 of GenBank Accession No. NP_000576.1.

SEQ ID NO: 8 is a SARS-CoV-2 S protein coding sequence. The coding sequence for SARS-CoV-2 S1 is nt 46 to 2055 (underlined), which can be used to generate an mRNA vaccine for SARS-CoV-2 S1. The coding sequence for SARS-CoV-2 S2 is nt 2059 to 3624 (bold), which can be used to generate an mRNA vaccine for SARS-CoV-2 S2.

atgtttgtttttcttgttttattgccactagtctctagtcagtgtgtta
atcttacaaccagaactcaattaccccctgcatacactaattctttcac
acgtggtgtttattaccctgacaaagttttcagatcctcagttttacat
tcaactcaggacttgttcttacctttcttttccaatgttacttggttcc
atgctatacatgtctctgggaccaatggtactaagaggtttgataaccc
tgtcctaccatttaatgatggtgtttattttgcttccactgagaagtct
aacataataagaggctggatttttggtactactttagattcgaagaccc
agtccctacttattgttaataacgctactaatgttgttattaaagtctg
tgaatttcaattttgtaatgatccatttttgggtgtttattaccacaaa
aacaacaaaagttggatggaaagtgagttcagagtttattctagtgcga
ataattgcacttttgaatatgtctctcagccttttcttatggaccttga
aggaaaacagggtaatttcaaaaatcttagggaatttgtgtttaagaat
attgatggttattttaaaatatattctaagcacacgcctattaatttag
tgcgtgatctccctcagggtttttcggctttagaaccattggtagattt
gccaataggtattaacatcactaggtttcaaactttacttgctttacat
agaagttatttgactcctggtgattcttcttcaggttggacagctggtg
ctgcagcttattatgtgggttatcttcaacctaggacttttctattaaa
atataatgaaaatggaaccattacagatgctgtagactgtgcacttgac
cctctctcagaaacaaagtgtacgttgaaatccttcactgtagaaaaag
gaatctatcaaacttctaactttagagtccaaccaacagaatctattgt
tagatttcctaatattacaaacttgtgcccttttggtgaagtttttaac
gccaccagatttgcatctgtttatgcttggaacaggaagagaatcagca
actgtgttgctgattattctgtcctatataattccgcatcattttccac
ttttaagtgttatggagtgtctcctactaaattaaatgatctctgcttt
actaatgtctatgcagattcatttgtaattagaggtgatgaagtcagac
aaatcgctccagggcaaactggaaagattgctgattataattataaatt
accagatgattttacaggctgcgttatagcttggaattctaacaatctt
gattctaaggttggtggtaattataattacctgtatagattgtttagga
agtctaatctcaaaccttttgagagagatatttcaactgaaatctatca
ggccggtagcacaccttgtaatggtgttgaaggttttaattgttacttt
cctttacaatcatatggtttccaacccactaatggtgttggttaccaac
catacagagtagtagtactttcttttgaacttctacatgcaccagcaac
tgtttgtggacctaaaaagtctactaatttggttaaaaacaaatgtgtc
aatttcaacttcaatggtttaacaggcacaggtgttcttactgagtcta
acaaaaagtttctgcctttccaacaatttggcagagacattgctgacac
tactgatgctgtccgtgatccacagacacttgagattcttgacattaca
ccatgttcttttggtggtgtcagtgttataacaccaggaacaaatactt
ctaaccaggttgctgttctttatcaggatgttaactgcacagaagtccc
tgttgctattcatgcagatcaacttactcctacttggcgtgtttattct
acaggttctaatgtttttcaaacacgtgcaggctgtttaataggggctg
aacatgtcaacaactcatatgagtgtgacatacccattggtgcaggtat
atgcgctagttatcagactcagactaattctcctcggcgggcacgtagt
gtagctagtcaatccatcattgcctacactatgtcacttggtgcagaaa
attcagttgcttactctaataactctattgccatacccacaaattttac
tattagtgttaccacagaaattctaccagtgtctatgaccaagacatca
gtagattgtacaatgtacatttgtggtgattcaactgaatgcagcaatc
ttttgttgcaatatggcagtttttgtacacaattaaaccgtgctttaac
tggaatagctgttgaacaagacaaaaacacccaagaagtttttgcacaa
gtcaaacaaatttacaaaacaccaccaattaaagattttggtggtttta
atttttcacaaatattaccagatccatcaaaaccaagcaagaggtcatt
tattgaagatctacttttcaacaaagtgacacttgcagatgctggcttc
atcaaacaatatggtgattgccttggtgatattgctgctagagacctca
tttgtgcacaaaagtttaacggccttactgttttgccacctttgctcac
agatgaaatgattgctcaatacacttctgcactgttagcgggtacaatc
acttctggttggacctttggtgcaggtgctgcattacaaataccatttg
ctatgcaaatggcttataggtttaatggtattggagttacacagaatgt
tctctatgagaaccaaaaattgattgccaaccaatttaatagtgctatt
ggcaaaattcaagactcactttcttccacagcaagtgcacttggaaaac
ttcaagatgtggtcaaccaaaatgcacaagctttaaacacgcttgttaa
acaacttagctccaattttggtgcaatttcaagtgttttaaatgatatc
ctttcacgtcttgacaaagttgaggctgaagtgcaaattgataggttga
tcacaggcagacttcaaagtttgcagacatatgtgactcaacaattaat
tagagctgcagaaatcagagcttctgctaatcttgctgctactaaaatg
tcagagtgtgtacttggacaatcaaaaagagttgatttttgtggaaagg
gctatcatcttatgtccttccctcagtcagcacctcatggtgtagtctt
cttgcatgtgacttatgtccctgcacaagaaaagaacttcacaactgct
cctgccatttgtcatgatggaaaagcacactttcctcgtgaaggtgtct
ttgtttcaaatggcacacactggtttgtaacacaaaggaatttttatga
accacaaatcattactacagacaacacatttgtgtctggtaactgtgat
gttgtaataggaattgtcaacaacacagtttatgatcctttgcaacctg
aattagactcattcaaggaggagttagataaatattttaagaatcatac
atcaccagatgttgatttaggtgacatctctggcattaatgcttcagtt
gtaaacattcaaaaagaaattgaccgcctcaatgaggttgccaagaatt
taaatgaatctctcatcgatctccaagaacttggaaagtatgagcagta
tataaaatggccatggtacatttggctaggttttatagctggcttgatt
gccatagtaatggtgacaattatgctttgctgtatgaccagttgctgta
gttgtctcaagggctgttgttcttgtggatcctgctgcaaatttgatga
agacgactctgagccagtgctcaaaggagtcaaattacattacacataa

SEQ ID NO: 9 is a native SARS-CoV-2 S2 subunit protein sequence.

SEQ ID NO: 10 is an exemplary CpG sequence that can be used in the compositions, nanoparticles, and methods herein.

SEQ ID NO: 11 is a SARS-CoV-2 S protein sequence from the omicron variant (variant B.1.1.529), including the signal peptide aa 1-15 which includes at least the following mutations A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F) (numbering based on SEQ ID NO: 1).

SEQ ID NO: 12 is a SARS-CoV-2 S1 protein sequence from the omicron variant (variant B.1.1.529).

SEQ ID NO: 13 is a SARS-CoV-2 S2 protein sequence from the omicron variant (variant B.1.1.529).

SEQ ID NO: 14 is a SARS-CoV-2 S protein sequence from the delta variant (variant B.1.617.2), including the signal peptide aa 1-15, which includes at least the following mutations T19R, G142D, E156G, 157-158 deletion, L452R, T478K, D614G, P681R, D950N) (numbering based on SEQ ID NO: 1). (NCBI Ref. No. MZ437368.1, incorporated by reference herein).

SEQ ID NO: 15 is a SARS-CoV-2 S2 protein sequence from the delta variant (variant B.1.617.2).

SEQ ID NO: 16 is a SARS-CoV-2 S2 protein sequence from the delta variant (variant B.1.617.2).

### DETAILED DESCRIPTION

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Adjuvant: A component of an immunogenic composition (or nanoparticle provided herein) used to enhance antigenicity. In some embodiments, an adjuvant can include a suspension of minerals (e.g., those that include aluminum (alum), such as aluminum sulfate, aluminum potassium sulfate, aluminum hydroxide, or aluminum phosphate, or include phosphate) on which antigen (such as a SARS-CoV-2 S1 subunit) is adsorbed; or water-in-oil emulsion, for example, in which antigen solution is emulsified in mineral oil (Freund's incomplete adjuvant), sometimes with the inclusion of killed mycobacteria (Freund's complete adjuvant) to further enhance antigenicity (inhibits degradation of antigen and/or causes influx of macrophages). In some embodiments, the adjuvant used in a disclosed immunogenic composition or nanoparticle is a combination of lecithin and carbomer homopolymer (such as the ADJUPLEX™ adjuvant available from Advanced BioAdjuvants, LLC; see also Wegmann, Clin Vaccine Immunol 22(9): 1004-1012, 2015). Additional adjuvants for use in the disclosed immunogenic compositions or nanoparticles include the QS21 purified plant extract, Matrix M, AS01, MF59, and ALFQ adjuvants.

Immunostimulatory oligonucleotides (such as those including a CpG motif) can also be used as adjuvants. In one example, adjuvants for use in the disclosed immunogenic compositions or nanoparticles include a CpG oligonucleotide, IL15 (such as human IL-15, such as SEQ ID NO: 7), and polyinosine-polycytidic acid (poly(I:C)). Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. Additional exemplary adjuvants can be found in Singh (ed.) Vaccine Adjuvants and Delivery Systems. Wiley-Interscience, 2007).

Administration: The introduction of an agent, such as a disclosed immunogenic composition and disclosed nanoparticles, into a subject by a chosen route. Administration can be local or systemic. For example, if the chosen route is intranasal (IN), the agent (such as an immunogen including a SARS-CoV-2 S protein, S1 protein, or S2 protein) is administered by introducing the composition into the nasal passages of the subject. In one example, administration is intramuscular (IM). Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide (such as a SARS-CoV-2 S protein, S1 protein, or S2 protein) with a different amino acid. In one example, a SARS-CoV-2 S protein includes K986P and V987P amino acid substitutions.

Carrier: An immunogenic molecule to which an antigen (such as a SARS-CoV-2 S protein, S1 protein, or S2 protein) can be linked. When linked to a carrier, the antigen may become more immunogenic. Carriers increase the immunogenicity of the antigen and/or to elicit antibodies against the carrier which are diagnostically, analytically, and/or therapeutically beneficial. Useful carriers include polymeric carriers, which can be natural (for example, proteins from bacteria or viruses), semi-synthetic or synthetic materials containing one or more functional groups to which a reactant moiety can be attached.

Conservative variants: “Conservative” amino acid substitutions are those substitutions that do not substantially affect or decrease a function of a protein (such as a SARS-CoV-2 S protein, S1 protein, or S2 protein), such as the ability of the protein to induce an immune response when administered to a subject. The term conservative variation also includes the use of a substituted amino acid in place of an unsubstituted parent amino acid. Furthermore, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some examples, non-conservative substitutions alter an activity or function of a SARS-CoV-2 S protein, S1 protein, or S2 protein, such as the ability to induce an immune response when administered to a subject. For instance, if an amino acid residue is essential for a function of the protein, even an otherwise conservative substitution may disrupt that activity. Thus, a conservative substitution does not alter the basic function of a protein of interest.

Control: A reference standard. In some embodiments, the control is a negative control sample obtained from a healthy patient. In other embodiments, the control is a positive control sample obtained from a patient diagnosed with a SARS-CoV-2 infection, such as COVID-19. In still other embodiments, the control is a historical control or standard reference value or range of values (such as a previously tested control sample, such as a group of patients infected with a SARS-CoV-2 with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Coronavirus: A large family of positive-sense, single-stranded RNA viruses that can infect humans and non-human animals. Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-CoV), SARS-CoV-2, and Middle East respiratory syndrome coronavirus (MERS-CoV). Other coronaviruses that infect humans include human coronavirus HKU1 (HKU1-CoV), human coronavirus OC43 (OC43-CoV), human coronavirus 229E (229E-CoV), and human coronavirus NL63 (NL63-CoV).

COVID-19: A contagious disease caused by severe acute respiratory syndrome coronavirus 2 (SARS-CoV-2). Symptoms of COVID-19 are variable, but often include fever, cough, fatigue, breathing difficulties, and loss of smell and taste. Symptoms can begin one to fourteen days after exposure to the virus. Some individuals do not develop any symptoms. While most people have mild symptoms, some people develop acute respiratory distress syndrome (ARDS). ARDS can be precipitated by cytokine storms, multi-organ failure, septic shock, and blood clots. Longer-term damage to organs (in particular, the lungs and heart) has been observed. There is concern about a significant number of patients who have recovered from the acute phase of the disease but continue to experience a range of effects—known as long COVID—for months afterwards. These effects include severe fatigue, memory loss and other cognitive issues, low-grade fever, muscle weakness, and breathlessness.

Effective amount: An amount of agent, such as an immunogenic composition as described herein, that is sufficient to elicit a desired response, such as an immune response in a subject. It is understood that to obtain a protective immune response against an antigen of interest (such as SARS-CoV-2 S protein, S1 protein, or S2 protein) can require multiple administrations of a disclosed immunogen, and/or administration of a disclosed immunogen as the “prime” in a prime boost protocol wherein the boost immunogen can be different from the prime immunogen. Accordingly, an effective amount of a disclosed immunogen can be the amount of the immunogen sufficient to elicit a priming immune response in a subject that can be subsequently boosted with the same or a different immunogen to elicit a protective immune response. In some examples, an effective amount of a disclosed immunogen is an amount effective to induce a “boost” response.

In one example, a desired response is to inhibit or reduce or prevent SARS-CoV-2 infection. The SARS-CoV-2 infection does not need to be completely eliminated or reduced or prevented for the method to be effective. For example, administration of an effective amount of the agent can induce an immune response that decreases the SARS-CoV-2 infection (for example, as measured by infection of cells, or by number or percentage of subjects infected by SARS-CoV-2), for example by at least 50%, at least 60%, at least 70%, at least 80%, at least 90%, at least 95%, at least 98%, or even at least 100% (elimination or prevention of detectable SARS-CoV-2 infection), as compared to a suitable control.

In one example, a desired response is to inhibit or reduce or prevent SARS-CoV-2 transmission to another subject. The SARS-CoV-2 transmission does not need to be completely eliminated or reduced or prevented for the method to be effective. For example, administration of an effective amount of the agent can induce an immune response in one subject, that decreases SARS-CoV-2 transmission to another subject (for example, as measured by infection of cells in the other subject, or by number or percentage of subjects infected by SARS-CoV-2), for example by at least 50%, at least 60%, at least 70%, at least 80%, at least 90%, at least 95%, at least 98%, or even at least 100% (elimination or prevention of detectable SARS-CoV-2 transmission), as compared to a suitable control.

In some examples, combinations of these effects are obtained.

Heterologous: Originating from a separate genetic source or species. For example, a heterologous polypeptide or polynucleotide refers to a polypeptide or polynucleotide derived from a different source or species.

Immune response: A response of a cell of the immune system, such as a B cell, T cell, or monocyte, to a stimulus. In some embodiments, the response is specific for a particular antigen (an “antigen-specific response”), such as a SARS-CoV-2 S1 protein (such as SEQ ID NO: 2, 3, 4, 5, 6, 12 or 15), SARS-CoV-2 S protein (such as SEQ ID NO: 1, 11 or 14, or to amino acids 16-1208 of SEQ ID NO: 1, amino acids 16-1205 of SEQ ID NO: 11, or amino acids 16-1206 of SEQ ID NO: 14, such as an S protein including K986P and V987P substitutions), or SARS-CoV-2 S2 protein (such as SEQ ID NO: 9, 13, or 16). In some embodiments, the immune response is a T cell response, such as a CD4+ response or a CD8+ response. In other embodiments, the response is a B cell response, and results in the production of specific antibodies. “Priming an immune response” refers to treatment of a subject with a “prime” immunogen/immunogenic composition to induce an immune response that is subsequently “boosted” with a boost immunogen/immunogenic composition, wherein in some examples the “prime” and “boost” immunogens are the same, but may include different adjuvants and may be administered differently (e.g., prime dose administered IM, and booster(s) administered IN). Together, the prime and boost immunizations produce the desired immune response in the subject.

Immunogen: A compound, composition, or substance (for example, a SARS-CoV-2 S1 protein, such as SEQ ID NO: 2, 3, 4, 5, 6, 12 or 15; SARS-CoV-2 S protein, such as SEQ ID NO: 1, 11 or 14, or to amino acids 16-1208 of SEQ ID NO: 1, amino acids 16-1205 of SEQ ID NO: 11, or amino acids 16-1206 of SEQ ID NO: 14, or an S protein including K986P and V987P substitutions; or SARS-CoV-2 S2 protein, such as SEQ ID NO: 9, 13 or 16) that can elicit an immune response in an animal, including compositions that are administered into an animal. Administration of an immunogen to a subject can lead to protective immunity against a pathogen of interest.

Immunogenic composition: A composition that includes an immunogen or a nucleic acid molecule or vector encoding an immunogen (such as a SARS-CoV-2 S protein, S1 protein, or S2 protein or mRNA encoding such), that elicits a measurable CTL response against the immunogen, and/or elicits a measurable B cell response (such as production of antibodies) against the immunogen, when administered to a subject. It further refers to isolated nucleic acids encoding an immunogen, such as a nucleic acid that can be used to express the immunogen (and thus be used to elicit an immune response against this immunogen). In some examples, for in vivo use, immunogenic compositions can include a SARS-CoV-2 S1 protein (such as one having at least 90% sequence identity to SEQ ID NO: 2, 3, 4, 5, 6, 12 or 15), a nucleic acid molecule encoding a SARS-CoV-2 S1 protein (such as a nucleic acid molecule encoding a protein having at least 90% sequence identity to SEQ ID NO: 2, 3, 4, 5, 6, 12 or 15, such as a nucleic acid molecule having at least 90% sequence identity to nt 46 to 2055 of SEQ ID NO: 8), a SARS-CoV-2 S protein (such as SEQ ID NO: 1, 11 or 14, or to amino acids 16-1208 of SEQ ID NO: 1, amino acids 16-1205 of SEQ ID NO: 11, or amino acids 16-1206 of SEQ ID NO: 14, such as an S protein including K986P and V987P substitutions), a nucleic acid molecule encoding a SARS-CoV-2 S protein (such as a nucleic acid molecule encoding a protein having at least 90% sequence identity to SEQ ID NO: 1, 11 or 14, or to amino acids 16-1208 of SEQ ID NO: 1, amino acids 16-1205 of SEQ ID NO: 11, or amino acids 16-1206 of SEQ ID NO: 14, such as a nucleic acid molecule having at least 90% sequence identity to SEQ ID NO: 8), SARS-CoV-2 S2 protein (such as SEQ ID NO: 9), or a nucleic acid molecule encoding a SARS-CoV-2 S2 protein (such as a nucleic acid molecule encoding a protein having at least 90% sequence identity to SEQ ID NO: 9, 13, or 16, such as a nucleic acid molecule having at least 90% sequence identity to nt 2059 to 3624 of SEQ ID NO: 8), in a pharmaceutically acceptable carrier and may also include other agents, such as one or more adjuvants.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Lyophilized: Lyophilization (also known as freeze drying) is a process by which water is removed from a material (such as a nucleic acid molecule or protein, such as a SARS-CoV-2 protein or nucleic acid molecule) after it is frozen and placed under a vacuum, allowing the ice to change directly from solid to vapor without passing through a liquid phase. The lyophilization process can include three separate processes: freezing, primary drying (sublimation), and secondary drying (desorption). Lyophilization is commonly used to preserve perishable materials, such as nucleic acids and proteins, to extend shelf life or make the material more convenient for transport.

Nanoparticle (NP): A particle of matter that is about 1 to 200 nanometres (nm) in diameter, such as about 50 to 90 nm or 70 to 90 nm in diameter. Nanoparticles are similar in size to viruses, such that when they are endocytosed into dendritic cells, molecules associated with the nanoparticle (such as a SARS-CoV-2 S protein, S1 protein, or S2 protein, or mRNA encoding such) are presented to B cell and T cells. Thus, nanoparticles can thus be loaded with antigen, such as a SARS-CoV-2 S protein, S1 protein, or S2 protein, and other reagents, such as an adjuvant.

In some examples, a nanoparticle is polymeric, such as a nanocapsule or nanosphere. In some examples, nanoparticles are composed of PLGA, poly(F-caprolactone) (PCL), poly(ethylene glycol) (PEG), poly(lactic acid) (PLA) or combinations thereof. In some examples, nanoparticles are composed of lipids (LNPs), such as 1,2-dilinoleyloxy-3-dimethylaminopropane (DOPE), 1,2-dioleoyl-sn-glycero-3-phosphoethanolamine (DOTAP), 1,2-Dioleyl-3-trimethylammonium-propane chloride salt (DSPC), 1,2-Distearoyl-sn-glycero-3-phosphocholine; Dipalmitoylphosphatidylcholine (PC), phosphatidylserine (PS), cholesterol, or combinations thereof. In a specific example, nanoparticles are composed of PLGA. In another specific example, nanoparticles are composed of DOTAP.

Nucleic acid molecule: A polymeric form of nucleotides, which may include both sense and anti-sense strands of RNA, mRNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens, (such as a SARS-CoV-2 S protein, S1 protein, S2 protein, or mRNA encoding such) and immunogenic compositions and nanoparticles containing such immunogens.

The nature of the carrier can depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate. In particular embodiments, suitable for administration to a subject the carrier may be sterile, and/or suspended or otherwise contained in a unit dosage form containing one or more measured doses of the composition suitable to elicit the desired anti-SARS-CoV-2 immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage. In some examples, the unit dosage form is in a sealed vial that contains sterile contents for intranasal administration to a subject.

Polypeptide: A chain of at least 10 amino acids, regardless of post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Preventing, treating or ameliorating a disease: “Preventing” a disease refers to inhibiting the full development of a disease, such as preventing infection by SARS-Cov-2. “Treating” refers to a therapeutic intervention that ameliorates a sign or symptom of a disease or pathological condition (such as COVID-19) after it has begun to develop, such as a reduction in viral load or sign/symptom of COVID-19. “Ameliorating” refers to the reduction in the number or severity of signs or symptoms of a disease, such as a SARS-CoV-2 infection.

Prime-boost vaccination: An immunotherapy including administration of a first immunogenic composition (the primary vaccine) followed by administration of a second immunogenic composition (the booster vaccine) to a subject to induce an immune response. In some examples, multiple booster vaccines are administered, such as at least 2, at least 3, at least 4 or at least 5 booster vaccines. The priming vaccine and/or the booster vaccine includes the antigen to which the immune response is directed (e.g., SARS-CoV-2 S protein, S1 or protein, or S2 protein), or a nucleic acid molecule (which may be part of a vector, such as a viral vector, RNA, or DNA vector) encoding the antigen to which the immune response is directed. The booster vaccine is administered to the subject after the priming vaccine; a suitable time interval between administration of the priming vaccine and the booster vaccine, and examples of such timeframes are disclosed herein. In some embodiments, the priming vaccine, the booster vaccine, or both primer vaccine and the booster vaccine additionally include one or more adjuvants. In some examples, the adjuvant used in the priming vaccine differs from the adjuvant used in the booster vaccine (e.g., alum in the primary, and a combination of CpG, poly I:C, and IL-15 in the booster(s)). In some embodiments, the priming vaccine is administered IM, and the booster vaccine(s), is administered IN. In one non-limiting example, the priming vaccine is a nucleic acid-based vaccine (or other vaccine based on gene delivery, such as DNA or mRNA), for example which is administered IM, and the booster vaccine is a protein nanoparticle based vaccine provided herein, for example which is administered IN.

Recombinant: A recombinant nucleic acid, protein, or virus is one that has a sequence that is not naturally occurring or has a sequence that is made by an artificial combination of two otherwise separated segments of sequence. This artificial combination can be accomplished, for example, by the artificial manipulation of isolated segments of nucleic acids, for example, using genetic engineering techniques.

SARS-CoV-2: Also known as Wuhan coronavirus, 2019-nCoV, or 2019 novel coronavirus, SARS-CoV-2 is a positive-sense, single stranded RNA virus of the genus betacoronavirus that has emerged as a highly fatal cause of severe acute respiratory infection. The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)—membrane (M) and nucleocapsid (N)—3′. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

In one example, a SARS-CoV-2 is a naturally occurring variant thereof, such as alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2), and omicron (B.1.1.529). S protein, S1 protein, S2 protein, or nucleic acids encoding such, from these SARS-CoV-2 variants, can be used as an immunogen for the compositions and nanoparticles provided herein. Thus, reference to a SARS-CoV-2 S protein can be an S protein from the native SARS-CoV-2, or from a variant thereof.

Standard methods for detecting viral infection may be used to detect SARS-CoV-2 infection, including but not limited to, assessment of patient symptoms and background, genetic tests such as reverse transcription-polymerase chain reaction (rRT-PCR), and antigen tests such as ELISA to detect SARS-CoV-2 proteins. The test can be done on patient samples such as respiratory or blood samples.

SARS-CoV-2 Spike (S): A class I fusion glycoprotein initially synthesized as a precursor protein of approximately 1270 amino acids in size. Individual precursor S polypeptides form a homotrimer and undergo glycosylation within the Golgi apparatus as well as processing to remove the signal peptide. The S polypeptide includes S1 and S2 proteins separated by a protease cleavage site between approximately amino acid positions 685/686 (e.g., of SEQ ID NO: 1). Cleavage at this site generates separate S1 and S2 polypeptide chains, which remain associated as S1/S2 protomers within the homotrimer. It is believed that the beta coronaviruses are generally not cleaved prior to the low pH cleavage that occurs in the late endosome-early lysosome by the TMPRSS2 protease, at the start of the fusion peptide. Cleavage between S1/S2 is not required for function and is not observed in all viral spikes. The S1 subunit is distal to the virus membrane and contains the receptor-binding domain (RBD) that is believed to mediate virus attachment to its host receptor. The S2 subunit is believed to contain the fusion protein machinery, such as the fusion peptide, two heptad-repeat sequences (HR1 and HR2) and a central helix typical of fusion glycoproteins, a transmembrane domain, and the cytosolic tail domain.

The amino acid numbering used in the disclosed SARS-CoV-2 S proteins and fragments thereof is relative to the S protein of SARS-CoV-2, the sequence of which is provided as SEQ ID NO: 1, and deposited as NCBI Ref. No. YP_009724390.1, which is incorporated by reference herein in its entirety. With reference to the SARS-CoV-2 S protein sequence provided as SEQ ID NO: 1, the ectodomain of the SARS-CoV-2S protein includes about residues 16-1208. Residues 1-15 are the signal peptide, which is removed during cellular processing. The S1/S2 cleavage site is located at position 685/686. The HR1 is located at about residues 915-983. The central helix is located at about residues 988-1029. The HR2 is located at about 1162-1194. The C-terminal end of the S2 ectodomain is located at about residue 1208. In some embodiments, the protomers of the prefusion-stabilized SARS-CoV-2 S ectodomain trimer can have a C-terminal residue of the C-terminal residue of the HR2 (e.g., position 1194), or the ectodomain (e.g., position 1208), or from one of positions 1194-1208. The position numbering of the S protein may vary between SARS-CoV-2 stains, but the sequences can be aligned to determine relevant structural domains and cleavage sites. It will be appreciated that a few residues (such as up to 10) on the N and C-terminal ends of the ectodomain can be removed or modified in the disclosed immunogens without decreasing the utility of the S ectodomain trimer as an immunogen.

The protomers of the recombinant SARS-CoV-2 S trimer in the NDV VLP comprise a SARS-CoV-2 S ectodomain that is stabilized in a prefusion conformation by one or more amino acid substitutions. In some embodiments, the immunogen includes a recombinant SARS-CoV-2 S ectodomain trimer comprising protomers comprising one or more (such as two, for example two consecutive) proline substitutions at or near the boundary between a HR1 domain and a central helix domain that stabilize the S ectodomain trimer in the prefusion conformation. In some embodiments, the SARS-CoV-2 S ectodomain trimer is stabilized in the prefusion conformation by K986P and V987P substitutions (“2P”) in the S ectodomain protomers in the trimer. In some embodiments, the SARS-CoV-2 S ectodomain trimer is stabilized in the prefusion conformation by one or two proline substitutions at positions D985, K986, or V987 of the S ectodomain protomers in the trimer. In some embodiments, the protomers of the recombinant SARS-CoV-2 S ectodomain trimer stabilized in the prefusion conformation by the one or more proline substitutions (such as K986P and V987P substitutions) comprise one or more additional modifications for stabilization in the prefusion conformation.

Reference to a SARS-CoV-2 S1 subunit can refer to the original native sequence shown in SEQ ID NO: 2, as well as newly identified variants thereof, such as SEQ ID NO: 3, 4, 5, 6, 12 and 15. An exemplary SARS-CoV-2 S1 subunit coding sequence is provided in nt 46-2055 of SEQ ID NO: 8.

Reference to a SARS-CoV-2 S2 subunit can refer to the original native sequence shown in SEQ ID NO: 9, as well as newly identified variants thereof, such as SEQ ID NOS: 13 and 16. In some examples, a SARS-CoV-2 S2 subunit includes K986P and V987P substitutions (numbers with reference to SEQ ID NO: 1). An exemplary SARS-CoV-2 S2 subunit coding sequence is provided in nt 2059 to 3624 of SEQ ID NO: 8.

Sequence identity: The similarity between amino acid or nucleotide sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide or polynucleotide will possess a relatively high degree of sequence identity when aligned using standard methods.

Methods of alignment of sequences for comparison are known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide or nucleic acid sequence are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid or nucleotide sequence of interest. Sequences with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids (or 30-60 nucleotides), and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence. Thus, an immunogenic composition that includes a SARS-CoV-2 S1 subunit comprising at least 90% sequence identity to SEQ ID NO: 2, 3, 4, 5, 6, 12 or 15 is one that includes a SARS-CoV-2 S1 subunit having at least 90%, at least 91%, at least 92%, at least 93%, at least 94%, at least 95%, at least 96%, at least 97%, at least 98%, at least 99%, or even 100% identity to SEQ ID NO: 2, 3, 4, 5, 6, 12 or 15, respectively. Similarly, an immunogenic composition that includes a SARS-CoV-2 S1 coding sequences that has at least 90% sequence identity to nt 46-2055 of SEQ ID NO: 8 is one having at least 90%, at least 91%, at least 92%, at least 93%, at least 94%, at least 95%, at least 96%, at least 97%, at least 98%, at least 99%, or even 100% identity to nt 46-2055 of SEQ ID NO: 8. An immunogenic composition that includes a SARS-CoV-2 S2 subunit comprising at least 90% sequence identity to SEQ ID NO: 9, 13, or 16 is one that includes a SARS-CoV-2 S2 subunit having at least 90%, at least 91%, at least 92%, at least 93%, at least 94%, at least 95%, at least 96%, at least 97%, at least 98%, at least 99%, or even 100% identity to SEQ ID NO: 9, 13 or 16, respectively. Similarly, an immunogenic composition that includes a SARS-CoV-2 S2 coding sequence that has at least 90% sequence identity to nt 2059 to 3624 of SEQ ID NO: 8 is one having at least 90%, at least 91%, at least 92%, at least 93%, at least 94%, at least 95%, at least 96%, at least 97%, at least 98%, at least 99%, or even 100% identity to nt 2059 to 3624 of SEQ ID NO: 8. An immunogenic composition that includes a SARS-CoV-2 S protein comprising at least 90% sequence identity to SEQ ID NO: 1, 11 or 14, or amino acids 16-1208 of SEQ ID NO: 1, amino acids 16-1205 of SEQ ID NO: 11, or amino acids 16-1206 of SEQ ID NO: 14, is one that includes a SARS-CoV-2 S protein having at least 90%, at least 91%, at least 92%, at least 93%, at least 94%, at least 95%, at least 96%, at least 97%, at least 98%, at least 99%, or even 100% identity to SEQ ID NO: 1, 11 or 14, or amino acids 16-1208 of SEQ ID NO: 1, amino acids 16-1205 of SEQ ID NO: 11, or amino acids 16-1206 of SEQ ID NO: 14, respectively. Similarly, an immunogenic composition that includes an SARS-CoV-2 S coding sequence that has at least 90% sequence identity to SEQ ID NO: 8 is one having at least 90%, at least 91%, at least 92%, at least 93%, at least 94%, at least 95%, at least 96%, at least 97%, at least 98%, at least 99%, or even 100% identity to SEQ ID NO: 8.

Signal Peptide: A short amino acid sequence (e.g., approximately 10-35 amino acids in length) that directs newly synthesized secretory or membrane proteins to and through membranes (for example, the endoplasmic reticulum membrane). Signal peptides are typically located at the N-terminus of a polypeptide and are removed by signal peptidases. Signal peptide sequences typically contain three common structural features: an N-terminal polar basic region (n-region), a hydrophobic core, and a hydrophilic c-region).

Subject: Living multicellular vertebrate organisms, a category that includes human and non-human mammals. In some embodiments, the subject is a human. In some embodiments, the subject is a non-human primate (such as a macaque). In some embodiments, the subject is a hamster. In some embodiments, the subject is a ferret. In some examples the subject is a cat or dog. In some examples, a subject who is in need of inhibiting or preventing a SARS-CoV-2 infection is selected. For example, the subject can be uninfected and at risk of SARS-CoV-2 infection.

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a peptide (such as a SARS-CoV-2 S protein, S1 protein, or S2 protein), a polynucleotide (such as a nucleic acid encoding a SARS-CoV-2 protein, S1 protein, or S2 protein, such as an mRNA), a virus, a cell or one or more cellular constituents. In one specific example, a vaccine includes a recombinant SARS-CoV-2 S1 protein, or mRNA encoding such. In one specific example, a vaccine includes a recombinant SARS-CoV-2 S2 protein, or mRNA encoding such. In one specific example, a vaccine includes a recombinant SARS-CoV-2 S protein, or mRNA encoding such. In one specific, non-limiting example, a vaccine reduces the severity of the symptoms associated with SARS-CoV-2 infection and/or decreases the viral load compared to a control. In another non-limiting example, a vaccine reduces SARS-CoV-2 infection and/or transmission compared to a control. In another non-limiting example, a vaccine reduces one or more symptoms of COVID-19 compared to a control. In some examples, the control is a subject or population who did not receive the vaccine.

Vector: An entity containing a DNA or RNA molecule bearing a promoter(s) that is operationally linked to the coding sequence of a protein (such as an immunogenic protein, such as a SARS-CoV-2 S protein, S1 protein, or S2 protein) and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. Non-limiting examples of viral vectors include adenovirus vectors, adeno-associated virus (AAV) vectors, and poxvirus vectors (e.g., vaccinia, fowlpox).

### Overview

Emerging of SARS-CoV-2 variants and waning of vaccine/infection-induced immunity poses threats to curbing the COVID-19 pandemic. An effective, safe, and convenient booster vaccine is needed. While most vaccine strategies have focused on systemic immunization, the present disclosure provides data showing the protective efficacy of an intramuscular (IM) vaccine and an IM-primed and intranasal (IN)-boosted mucosal vaccine with SARS-CoV-2 spike protein S1 in three animal models. The IM-alum-only vaccine induced robust binding and neutralizing antibody and persistent cellular immunity systemically and mucosally, while IN boosting with nanoparticles including IL-15 and TLR agonists elicited weaker T-cell and antibody responses, but higher dimeric IgA and IFNα. Nevertheless, following SARS-CoV-2 challenge, neither group showed detectable subgenomic RNA in upper or lower respiratory tracts vs naïve controls, demonstrating sterilizing protection. The results demonstrate both vaccines can protect against respiratory SARS-CoV-2 exposure. The disclosed mucosal vaccine, which was safe after multiple doses and can clear the input virus more efficiently in the nasal cavity, may act as a potent reinforcing boost for conventional systemic vaccines to provide overall better protection.

The inventors demonstrated that by inducing mucosal antibody and T-cell immunity, as well as innate immunity, the disclosed mucosal vaccines can prevent or abort infection locally at the site of transmission before the virus disseminates systemically. This may be critical also because once the virus disseminates systemically, it can cause damage to other organs and widespread coagulopathies. The need for mucosal immunity and mucosal vaccines for SARS-CoV-2 has been emphasized. The immunogenicity and protective efficacy of two vaccines, one systemic and one mucosal, were compared in the rhesus macaque model. In some embodiments, the systemic strategy is an intramuscularly (IM) administered vaccine composed of SARS-CoV-2 S1 protein adjuvanted with alum. In some examples, the mucosal strategy is a mucosal vaccine primed with IM-alum and boosted with intranasally-administered spike protein nanoparticles adjuvanted with TLR agonists and IL-15. The protective efficacy was determined after 3 or 4 doses. These two subunit vaccines mediate 100% protection against subgenomic viral RNA from SARS-CoV-2 viral challenges in the upper and lower respiratory tracts, rarely achieved with other COVID-19 vaccines in macaques. Moreover, the rapid clearance of genomic RNA from the upper respiratory tract indicates an ability to reduce risk of transmission to others.

The adjuvanted subunit vaccines have important clinical implications. While the Emergency Use Authorization (EUA) of two mRNA vaccines and an adenoviral vector vaccine has been granted and others are about to be approved, third and more boosts are be needed to induce long-lasting protective immunity after the waning of the induced antibody responses. The disclosed materials provide a more effective boost because of the unique efficacy of the mucosally delivered nanoparticles. However, the common occurrence of adverse events (higher than 50%) after the administration of mRNA vaccines, especially the finding that adverse events were more common and the reactogenicity was generally greater after the second dose, raises the concern whether additional boosters of the same mRNA or adenovirus vaccine will be well tolerated or appropriate. Furthermore, the requirement for stringent cold-chain storage/transfer poses problems for mRNA vaccines. Thus in some examples the disclosed vaccines, immunogenic compositions, or nanoparticles do not require storage at −80° C., and in some examples can be stored at −20° C. (e.g., a typical freezer) or 4° C. (e.g., a typical refrigerator). Given the importance of respiratory mucosal immunity, the disclosed subunit mucosal vaccines provide a potent and complementary reinforcement for any systemically induced immunity.

The vaccine platforms provided herein mediate sterilizing protection against SARS-CoV-2 viral replication in macaque models, which has been rarely achieved. Furthermore, the mucosal vaccine containing nanoparticles appear more efficient at rapidly clearing the input virus (gRNA) in the upper and lower respiratory tracts than the systemic counterpart, providing a potent strategy to prevent viral transmission. However, since the protection against sgRNA was so complete, potential immune correlates for the sterilizing immunity against replicating sgRNA were not assessed, but two correlates of clearance of input challenge virus (gRNA) were identified (BAL IgA and IFN-α, induced by the mucosal immunization).

The mucosal vaccine containing nanoparticles (CP15-IN) provided herein mediated full protection in both the lower lung and nasal cavity with relatively low neutralizing antibody (Nab) titers, indicating complementary additional protective mechanisms. Even though early Nab titers were comparable (Alum-IM) or not as good (CP15-IN) as those of the mRNA vaccine, the last boost increase the Nab titers higher (Alum-IM) or to a comparable level (CP15-IN) to that of the mRNA vaccine. Compared to macaques vaccinated with 10 and 100 μg of mRNA-1273, which induced Nab titers of 501 and 3481 respectively, after the last boost, the mucosal vaccine described here induced a Nab titer of 374. Yet, the disclosed mucosal vaccine containing nanoparticles (CP15-IN) demonstrated outstanding protection in both upper and low respiratory tracts, especially in the nasal cavity, where 0/6 animals had detectable viral sgRNAs, a measure of viable replicating virus, and only 1/6 had detectable viral gRNAs, a measure of residual challenge virus, two days after viral challenge. With the systemic vaccine, which induced a much higher Nab titer (more than 4047), 3 of 6 animals had gRNA in their nasal swabs. Therefore, Nab titer is not the only protective mechanism for virus clearance.

Two parameters might account for the complete protection against viral replication (sgRNA) by the mucosal vaccine without high titers of antibody or T cell responses. The mucosal vaccine induced a qualitatively different response in the lung, with more dimeric IgA compared to monomeric IgA. This qualitative difference might outweigh the quantity of IgA or IgG measured. Another parameter was the higher frequency of CD14⁻/CD16⁺ cells in the lung after boosting via the mucosal route, which was associated with higher production of IFN-α upon restimulation with a viral infection mimic (S1 protein+poly I:C dsRNA). IFN-α and/or dimeric IgA may be critical for the mucosally vaccinated animals to control viral replication and rapidly clear input virus, especially at the mucosal surface. Thus, these results indicate that the qualitatively different responses induced in the lung by the mucosal vaccine boosts may complement immunity induced by conventional systemic vaccines against respiratory virus transmission.

The disclosed mucosal vaccine containing nanoparticles (CP15-IN) can be used for booster doses, especially to induce complementary mucosal immunity. Furthermore, the disclosed mucosal vaccine containing nanoparticles (CP15-IN) can be used as boosters for other priming vaccines, such as administered subsequent to an IM administration of an mRNA based vaccine (such as Pfizer-BioNTech's BNT162b2 vaccine, or Moderna's mRNA-1273 vaccine). The disclosed vaccines appear safe, and no vaccine-induced immune pathology was observed even after 3 or 4 doses. In the macaque model, the fourth dose of the mucosal vaccine containing nanoparticles (CP15-IN) mediated sterile protection against viral challenges. The mucosal boost induces local respiratory mucosal protection and complements or synergizes with systemic immunity to improve overall protection. Local respiratory mucosal immunity that can clear the virus to which a person was exposed at the site of transmission before it disseminates systemically could also potentially prevent serious complications of COVID-19 such as blood clotting disorders and kidney, heart, liver and brain damage, and also prevent transmission to other individuals.

The immunogenicity and protective efficacy of the disclosed SARS-CoV-2 subunit booster was examined using beta-variant S1 with IL-15 and TLR agonists in previously immunized macaques (one year after the first vaccination), as a variant-modified mucosal booster vaccine might induce local immunity to prevent SARS-CoV-2 infection at the port of entry. The beta-variant was the hardest to cross-neutralize before the omicron variant arose. The macaques were first vaccinated with Wuhan strain S1 with the same adjuvant. One year later, negligibly detectable SARS-CoV-2-specific antibody remained. Nevertheless, the booster induced vigorous humoral immunity including serum- and bronchoalveolar lavage (BAL)-IgG, secretory nasal- and BAL-IgA, and neutralizing antibody against the original strain and/or beta variant. Beta-variant S1-specific CD4+ and CD8+ T cell responses were also elicited in PBMC and BAL. Following SARS-CoV-2 beta variant challenge, the vaccinated group demonstrated significant protection against viral replication in the upper and lower respiratory tracts, with almost full protection in the nasal cavity. The fact that one intranasal beta-variant booster administrated one year after the first vaccination provoked protective immunity against beta variant infections demonstrates that the disclosed compositions and nanoparticles can be administered IN for effective boosting.

Also provided herein are data using a hamster model. Hamsters are more sensitive to viral transmission and disease, and tend to show symptoms of disease (e.g., weight loss), while macaques tend to show fewer symptoms. It was observed that the IN mucosal vaccine mediated significant protection against SARS-CoV-2 challenge in hamsters, whereas the systemic vaccine only showed a trend of significant protection compared with naive controls.

The newly emerging SARS-CoV-2 variants, which could escape the vaccine- or infection-induced neutralizing activity, may reduce the vaccine efficacy. Thus, booster vaccines, which will be administrated as a third or more doses, are urgently needed. Heterologous boosts may be more effective. Here, the inventors demonstrated that the adjuvanted subunit vaccines can be used as a booster, for example with mucosal nanoparticle delivery. [For corresponding publications see Sui et al., JCI Insight. 2021; 6(10):e148494; and Sui et al., bioRxiv 2021.10.19.464990; doi: https://doi.org/10.1101/2021.10.19.464990 both herein incorporated by reference in their entireties.]

Immunogenic Compositions and Nanoparticles that Include SARS-CoV-2 S Protein

Disclosed herein are immunogenic compositions, and immunogenic nanoparticles (simply referred to herein as nanoparticles for simplicity), such as vaccines, which both include a SARS-CoV-2 spike (S) protein (or coding sequence thereof), and one or more adjuvants. In some examples the S protein is the full-length S protein (e.g., SEQ ID NO: 1, 11 or 14, amino acids 16-1208 of SEQ ID NO: 1, amino acids 16-1205 of SEQ ID NO: 11, or amino acids 16-1206 of SEQ ID NO: 14, which may include K986P and N987P substitutions) or a subunit thereof, such as S1 (e.g., SEQ ID NO: 2, 3, 4, 5, 6, 12 or 15) or S2 (e.g., SEQ ID NO: 9, 13 or 16). In some examples the vaccine includes an mRNA encoding the S full-length protein (e.g., SEQ ID NO: 8 or nt 46-3624 of SEQ ID NO: 8, which may encode K986P and N987P substitutions) or a subunit thereof, such as S1 (e.g., nt 46-2055 SEQ ID NO: 8) or S2 (e.g., nt 2059 to 3624 of SEQ ID NO: 8). In some examples the SARS-CoV-2 S protein, S1, or S2 protein or mRNA is from the original Wuhan virus, or a variant thereof, such as alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2), or omicron (B.1.1.529). As demonstrated in the examples, the disclosed immunogenic compositions and immunogenic nanoparticles produce a superior immune response to SARS-CoV-2 when mammalian subjects are first primed with a immunogenic composition that includes a SARS-CoV-2 S1 protein (e.g., SEQ ID NO: 2) and one or more adjuvants (e.g., alum, such as aluminum phosphate, such as Adju-Phos® adjuvant), and subsequently boosted with one or more doses of immunogenic nanoparticles comprising a SARS-CoV-2 S1 protein (e.g., SEQ ID NO: 2) and one or more adjuvants (e.g., CpG, Poly I:C, and IL-15).

Immunogenic compositions that include a SARS-CoV-2 S1 protein having at least 80%, least 85%, at least 90%, at least 95%, at least 96%, at least 97%, at least 98%, at least 99%, or 100% sequence identity to SEQ ID NO: 2, 3, 4, 5, 6, 12 or 15; and one or more adjuvants are provided. In some examples, the SARS-CoV-2 S1 protein has at least 90% sequence identity to SEQ ID NO: 2. In some examples, the SARS-CoV-2 S1 protein comprises or consists of the amino acid sequence of SEQ ID NO: 2. In some examples, the SARS-CoV-2 S1 protein comprises or consists of the amino acid sequence of SEQ ID NO: 3. In some examples, the SARS-CoV-2 S1 protein comprises or consists of the amino acid sequence of SEQ ID NO: 4. In some examples, the SARS-CoV-2 S1 protein comprises or consists of the amino acid sequence of SEQ ID NO: 5. In some examples, the SARS-CoV-2 S1 protein comprises or consists of the amino acid sequence of SEQ ID NO: 6. In some examples, the SARS-CoV-2 S1 protein comprises or consists of the amino acid sequence of SEQ ID NO: 12. In some examples, the SARS-CoV-2 S1 protein comprises or consists of the amino acid sequence of SEQ ID NO: 15. In some examples, instead of a SARS-CoV-2 S1 protein, the composition includes an mRNA encoding the SARS-CoV-2 S1 protein, such as a coding sequence (such as an mRNA) that has at least 90% sequence identity to nt 46-2055 of SEQ ID NO: 8, such as at least 91%, at least 92%, at least 93%, at least 94%, at least 95%, at least 96%, at least 97%, at least 98%, at least 99%, or even 100% identity to nt 46-2055 of SEQ ID NO: 8. In some examples, the one or more adjuvants include alum, such as aluminum phosphate (e.g., Adju-Phos® adjuvant). In some examples, the one or more adjuvants include CpG oligodeoxynucleotide, Poly I:C, IL-15, or combinations thereof, or CpG oligodeoxynucleotide, Poly I:C and IL-15. The disclosed immunogenic compositions can further include a pharmaceutically acceptable carrier such as saline or water. Also provided are containers that include a disclosed immunogenic composition, such as a glass or plastic vial, or a syringe. In some examples, the disclosed immunogenic compositions are formulated for intramuscular administration. In some examples, the disclosed immunogenic compositions are formulated for intranasal administration.

Immunogenic compositions that include a SARS-CoV-2 S2 protein having at least 80%, least 85%, at least 90%, at least 95%, at least 96%, at least 97%, at least 98%, at least 99%, or 100% sequence identity to SEQ ID NO: 9, 13 or 16; and one or more adjuvants are provided. In some examples, the SARS-CoV-2 S2 protein has at least 90% sequence identity to SEQ ID NO: 9, 13 or 16. In some examples, the SARS-CoV-2 S2 protein comprises or consists of the amino acid sequence of SEQ ID NO: 9, 13 or 16. In some examples, the SARS-CoV-2 S2 protein includes K986P and V987P substitutions (numbering based on SEQ ID NO: 1), and has at least 80%, least 85%, at least 90%, at least 95%, at least 96%, at least 97%, at least 98%, at least 99%, or 100% sequence identity to SEQ ID NO: 9, 13, or 16. In some examples, the SARS-CoV-2 S2 protein includes a D950N substitution (numbering based on SEQ ID NO: 1), and has at least 80%, least 85%, at least 90%, at least 95%, at least 96%, at least 97%, at least 98%, at least 99%, or 100% sequence identity to SEQ ID NO: 9, 13, or 16. In some examples, instead of a SARS-CoV-2 S2 protein, the composition includes an mRNA encoding the SARS-CoV-2 S2 protein, such as a coding sequence (such as an mRNA) that has at least 90% sequence identity to nt 2059 to 3624 of SEQ ID NO: 8, such as at least 91%, at least 92%, at least 93%, at least 94%, at least 95%, at least 96%, at least 97%, at least 98%, at least 99%, or even 100% identity to nt 2059 to 3624 of SEQ ID NO: 8. In some examples, the one or more adjuvants include alum, such as aluminum phosphate (e.g., Adju-Phos® adjuvant). In some examples, the one or more adjuvants include CpG oligodeoxynucleotide, Poly I:C, IL-15, or combinations thereof, or CpG oligodeoxynucleotide, Poly I:C and IL-15. The disclosed immunogenic compositions can further include a pharmaceutically acceptable carrier such as saline or water. Also provided are containers that include a disclosed immunogenic composition, such as a glass or plastic vial, or a syringe. In some examples, the disclosed immunogenic compositions are formulated for intramuscular administration. In some examples, the disclosed immunogenic compositions are formulated for intranasal administration.

Immunogenic compositions that include a SARS-CoV-2 S protein having at least 80%, least 85%, at least 90%, at least 95%, at least 96%, at least 97%, at least 98%, at least 99%, or 100% sequence identity to SEQ ID NO: 1, 11, 14, amino acids 16-1208 of SEQ ID NO: 1, amino acids 16-1205 of SEQ ID NO: 11, or amino acids 16-1206 of SEQ ID NO: 14, and one or more adjuvants are provided. In some examples, the SARS-CoV-2 S protein has at least 90% sequence identity to SEQ ID NO: 1 or amino acids 16-1208 of SEQ ID NO: 1. In some examples, the SARS-CoV-2 S protein has at least 90% sequence identity to SEQ ID NO: 11 or amino acids 16-1205 of SEQ ID NO: 11. In some examples, the SARS-CoV-2 S protein has at least 90% sequence identity to SEQ ID NO: 14 or amino acids 16-1206 of SEQ ID NO: 14. In some examples, the SARS-CoV-2 S protein comprises or consists of the amino acid sequence of SEQ ID NO: 1 or amino acids 16-1208 of SEQ ID NO: 1. In some examples, the SARS-CoV-2 S protein comprises or consists of the amino acid sequence of SEQ ID NO: 11, or amino acids 16-1205 of SEQ ID NO: 11. In some examples, the SARS-CoV-2 S protein comprises or consists of the amino acid sequence of SEQ ID NO: 14 or amino acids 16-1206 of SEQ ID NO: 14. In some examples, the SARS-CoV-2 S protein includes K986P and V987P substitutions (numbering based on SEQ ID NO: 1), and has at least 80%, least 85%, at least 90%, at least 95%, at least 96%, at least 97%, at least 98%, at least 99%, or 100% sequence identity to SEQ ID NO: 1. In some examples, instead of a SARS-CoV-2 S protein, the composition includes an mRNA encoding the SARS-CoV-2 S protein, such as a coding sequence (such as an mRNA) that has at least 90% sequence identity to SEQ ID NO: 8 or nt 46 to 3624 of SEQ ID NO: 8, such as at least 91%, at least 92%, at least 93%, at least 94%, at least 95%, at least 96%, at least 97%, at least 98%, at least 99%, or even 100% identity to SEQ ID NO: 8 or nt 46 to 3624 of SEQ ID NO: 8. In some examples, the one or more adjuvants include alum, such as aluminum phosphate (e.g., Adju-Phos® adjuvant). In some examples, the one or more adjuvants include CpG oligodeoxynucleotide, Poly I:C, IL-15, or combinations thereof, or CpG oligodeoxynucleotide, Poly I:C and IL-15. The disclosed immunogenic compositions can further include a pharmaceutically acceptable carrier such as saline or water. Also provided are containers that include a disclosed immunogenic composition, such as a glass or plastic vial, or a syringe. In some examples, the disclosed immunogenic compositions are formulated for intramuscular administration. In some examples, the disclosed immunogenic compositions are formulated for intranasal administration.

Nanoparticles that include a SARS-CoV-2 S1 protein having at least 80%, least 85%, at least 90%, at least 95%, at least 96%, at least 97%, at least 98%, at least 99%, or 100% sequence identity to SEQ ID NO: 2, 3, 4, 5, 6, 12 or 15; and one or more adjuvants are provided. In some examples, the SARS-CoV-2 S1 protein has at least 90% sequence identity to SEQ ID NO: 2. In some examples, the SARS-CoV-2 S1 protein comprises or consists of the amino acid sequence of SEQ ID NO: 2. In some examples, the SARS-CoV-2 S1 protein comprises or consists of the amino acid sequence of SEQ ID NO: 3. In some examples, the SARS-CoV-2 S1 protein comprises or consists of the amino acid sequence of SEQ ID NO: 4. In some examples, the SARS-CoV-2 S1 protein comprises or consists of the amino acid sequence of SEQ ID NO: 5. In some examples, the SARS-CoV-2 S1 protein comprises or consists of the amino acid sequence of SEQ ID NO: 6. In some examples, the SARS-CoV-2 S1 protein comprises or consists of the amino acid sequence of SEQ ID NO: 12. In some examples, the SARS-CoV-2 S1 protein comprises or consists of the amino acid sequence of SEQ ID NO: 15. In some examples, instead of a SARS-CoV-2 S1 protein, the nanoparticles include an mRNA encoding the SARS-CoV-2 S1 protein, such as a coding sequence (such as an mRNA) that has at least 90% sequence identity to nt 46-2055 of SEQ ID NO: 8, such as at least 91%, at least 92%, at least 93%, at least 94%, at least 95%, at least 96%, at least 97%, at least 98%, at least 99%, or even 100% identity to nt 46-2055 of SEQ ID NO: 8. In some examples, the one or more adjuvants include CpG oligodeoxynucleotide, Poly I:C, IL-15, or combinations thereof. In some examples, the one or more adjuvants include CpG oligodeoxynucleotide, Poly I:C and IL-15. In some examples, the IL-15 is human IL-15. The SARS-CoV-2 S1 protein or mRNA nanoparticle compositions can further include a pharmaceutically acceptable carrier such as saline or water. In some examples, the nanoparticle includes or is composed of PLGA. In some examples, the nanoparticle includes or is composed of lipids, such as a cationic lipid, such as DOTAP. The SARS-CoV-2 S1 protein or mRNA nanoparticles can be formulated into an immunogenic composition, such as one that further includes a pharmaceutically acceptable carrier such as saline or water. Also provided are containers that include the SARS-CoV-2 S1 protein or mRNA nanoparticle formulations, such as a glass or plastic vial, or a syringe. In some examples, the SARS-CoV-2 S1 protein or mRNA nanoparticles are formulated for mucosal vaccination, such as intranasal administration. Mucosal vaccination can be achieved by a number of routes including oral, intranasal, pulmonary, rectal and vaginal. In a specific example, this is achieved by intranasal administration. For example, the SARS-CoV-2 S1 protein or mRNA nanoparticle-containing compositions can include (or the nanoparticles themselves can include) one or more biodegradable, mucoadhesive polymeric carriers, such as PLGA, chitosan, alginate and carbopol.

Nanoparticles that include a SARS-CoV-2 S2 protein having at least 80%, least 85%, at least 90%, at least 95%, at least 96%, at least 97%, at least 98%, at least 99%, or 100% sequence identity to SEQ ID NO: 9, 13, or 16; and one or more adjuvants are provided. In some examples, the SARS-CoV-2 S2 protein has at least 90% sequence identity to SEQ ID NO: 9, 13, or 16. In some examples, the SARS-CoV-2 S1 protein comprises or consists of the amino acid sequence of SEQ ID NO: 9, 13, or 16. In some examples, the SARS-CoV-2 S2 protein includes K986P and V987P substitutions (numbering based on SEQ ID NO: 1), and has at least 80%, least 85%, at least 90%, at least 95%, at least 96%, at least 97%, at least 98%, at least 99%, or 100% sequence identity to SEQ ID NO: 9, 13 or 16. In some examples, instead of a SARS-CoV-2 S2 protein, the nanoparticles include an mRNA encoding the SARS-CoV-2 S2 protein, such as a coding sequence (such as an mRNA) that has at least 90% sequence identity to nt 2059 to 3624 of SEQ ID NO: 8, such as at least 91%, at least 92%, at least 93%, at least 94%, at least 95%, at least 96%, at least 97%, at least 98%, at least 99%, or even 100% identity to nt 2059 to 3624 of SEQ ID NO: 8. In some examples, the one or more adjuvants include CpG oligodeoxynucleotide, Poly I:C, IL-15, or combinations thereof. In some examples, the one or more adjuvants include CpG oligodeoxynucleotide, Poly I:C and IL-15. In some examples, the IL-15 is human IL-15. The SARS-CoV-2 S2 protein or mRNA nanoparticle compositions can further include a pharmaceutically acceptable carrier such as saline or water. In some examples, the nanoparticle includes or is composed of PLGA. In some examples, the nanoparticle includes or is composed of lipids, such as a cationic lipid, such as DOTAP. The SARS-CoV-2 S2 protein or mRNA nanoparticles can be formulated into an immunogenic composition, such as one that further includes a pharmaceutically acceptable carrier such as saline or water. Also provided are containers that include the SARS-CoV-2 S2 protein or mRNA nanoparticle formulations, such as a glass or plastic vial, or a syringe. In some examples, the SARS-CoV-2 S2 protein or mRNA nanoparticles are formulated for mucosal vaccination, such as intranasal administration. Mucosal vaccination can be achieved by a number of routes including oral, intranasal, pulmonary, rectal and vaginal. In a specific example, this is achieved by intranasal administration, which is the most relevant for SARS-CoV-2 that infects through the respiratory route. For example, the SARS-CoV-2 S2 protein or mRNA nanoparticle-containing compositions can include (or the nanoparticles themselves can include) one or more biodegradable, mucoadhesive polymeric carriers, such as PLGA, chitosan, alginate and carbopol.

Nanoparticles that include a SARS-CoV-2 S protein having at least 80%, least 85%, at least 90%, at least 95%, at least 96%, at least 97%, at least 98%, at least 99%, or 100% sequence identity to SEQ ID NO: 1, 11, 14, amino acids 16-1208 of SEQ ID NO: 1, amino acids 16-1205 of SEQ ID NO: 11, or amino acids 16-1206 of SEQ ID NO: 14, and one or more adjuvants are provided. In some examples, the SARS-CoV-2 S protein has at least 90% sequence identity to SEQ ID NO: 1 or amino acids 16-1208 of SEQ ID NO: 1. In some examples, the SARS-CoV-2 S protein has at least 90% sequence identity to SEQ ID NO: 11 or amino acids 16-1205 of SEQ ID NO: 11. In some examples, the SARS-CoV-2 S protein has at least 90% sequence identity to SEQ ID NO: 14 or amino acids 16-1206 of SEQ ID NO: 14. In some examples, the SARS-CoV-2 S protein comprises or consists of the amino acid sequence of SEQ ID NO: 1 or amino acids 16-1208 of SEQ ID NO: 1. In some examples, the SARS-CoV-2 S protein comprises or consists of the amino acid sequence of SEQ ID NO: 11, or amino acids 16-1205 of SEQ ID NO: 11. In some examples, the SARS-CoV-2 S protein comprises or consists of the amino acid sequence of SEQ ID NO: 14 or amino acids 16-1206 of SEQ ID NO: 14. In some examples, the SARS-CoV-2 S protein includes K986P and V987P substitutions (numbering based on SEQ ID NO: 1), and has at least 80%, least 85%, at least 90%, at least 95%, at least 96%, at least 97%, at least 98%, at least 99%, or 100% sequence identity to SEQ ID NO: 1. In some examples, instead of a SARS-CoV-2 S protein, the nanoparticles include an mRNA encoding the SARS-CoV-2 S protein, such as a coding sequence (such as an mRNA) that has at least 90% sequence identity to SEQ ID NO: 8 or nt 46 to 3624 of SEQ ID NO: 8, such as at least 91%, at least 92%, at least 93%, at least 94%, at least 95%, at least 96%, at least 97%, at least 98%, at least 99%, or even 100% identity to SEQ ID NO: 8 or nt 46 to 3624 of SEQ ID NO: 8. In some examples, the one or more adjuvants include CpG oligodeoxynucleotide, Poly I:C, IL-15, or combinations thereof. In some examples, the one or more adjuvants include CpG oligodeoxynucleotide, Poly I:C and IL-15. In some examples, the IL-15 is human IL-15. The SARS-CoV-2 S protein or mRNA nanoparticle compositions can further include a pharmaceutically acceptable carrier such as saline or water. In some examples, the nanoparticle includes or is composed of PLGA. In some examples, the nanoparticle includes or is composed of lipids, such as a cationic lipid, such as DOTAP. The SARS-CoV-2 S protein or mRNA nanoparticles can be formulated into an immunogenic composition, such as one that further includes a pharmaceutically acceptable carrier such as saline or water. Also provided are containers that include the SARS-CoV-2 S protein or mRNA nanoparticle formulations, such as a glass or plastic vial, or a syringe. In some examples, the SARS-CoV-2 S protein or mRNA nanoparticles are formulated for mucosal vaccination, such as intranasal administration. Mucosal vaccination can be achieved by a number of routes including oral, intranasal, pulmonary, rectal and vaginal. In a specific example, this is achieved by intranasal administration. For example, the SARS-CoV-2 S protein or mRNA nanoparticle-containing compositions can include (or the nanoparticles themselves can include) one or more biodegradable, mucoadhesive polymeric carriers, such as PLGA, chitosan, alginate and carbopol.

The disclosed immunogenic compositions and nanoparticles can be formulated with pharmaceutically acceptable carriers to help retain biological activity while also promoting increased stability during storage within an acceptable temperature range. Exemplary carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

In some examples, the disclosed immunogenic compositions and nanoparticles can be formulated with tromethamine, tromethamine hydrochloride, acetic acid, sodium acetate, potassium chloride, monobasic potassium phosphate, sodium chloride, dibasic sodium phosphate dehydrate, and/or polyethylene glycol.

The disclosed immunogenic compositions and nanoparticles, especially liquid formulations, can contain a bacteriostat to prevent or minimize degradation during storage, including but not limited to effective concentrations (usually 1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The disclosed immunogenic compositions and nanoparticles can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

In some instances it may be desirable to combine a disclosed immunogenic composition or nanoparticle formulation with other pharmaceutical products (e.g., vaccines) which induce protective responses to other agents. For example, a composition or nanoparticle including a SARS-CoV-2 S protein, S1 protein, S2 protein (or mRNA encoding such) can be administered simultaneously (typically separately) or sequentially with other vaccines recommended by the Advisory Committee on Immunization Practices (ACIP; cdc.gov/vaccines/acip/index.html) for the targeted age group (e.g., infants from approximately one to six months of age), such as an influenza vaccine or a varicella zoster vaccine. As such, a disclosed immunogenic composition or nanoparticle formulation including a SARS-CoV-2 S protein, S1 protein, S2 protein (or mRNA encoding such) may be administered simultaneously or sequentially with vaccines against, for example, hepatitis B (HepB), diphtheria, tetanus and pertussis (DTaP), pneumococcal bacteria (PCV), Haemophilus influenzae type b (Hib), polio, influenza or rotavirus.

In some embodiments, the disclosed immunogenic compositions and nanoparticles can be provided as a sterile composition. Typically, the amount of SARS-CoV-2 S protein, S1 protein, S2 protein (or mRNA encoding such) in each dose of the immunogenic composition or nanoparticle composition is selected as an amount which induces an immune response without significant, adverse side effects. In some embodiments, the composition can be provided in unit dosage form for use to induce an immune response in a subject. A unit dosage form contains a suitable single preselected dosage for administration to a subject, or suitable marked or measured multiples of two or more preselected unit dosages, and/or a metering mechanism for administering the unit dose or multiples thereof.

In one example, a disclosed immunogenic composition or nanoparticle-containing composition includes sodium alginate, which is a linear copolymer and consists of 1-4-linked β-d-mannuronic acid and 1-4-linked α-1-guluronic acid residues. In some examples, a disclosed immunogenic composition or nanoparticle-containing composition includes alginate microspheres. In one example, a disclosed immunogenic composition or nanoparticle-containing composition includes carbopol (a cross-linked polyacrylic acid polymer), for example in combination with starch. In some examples, a disclosed immunogenic composition or nanoparticle-containing composition includes chitosan, a non-toxic linear polysaccharide that can be produced by chitin deacetylation. In one example a nanoparticle is a chitosan nanoparticle, such as N-trimethyl chitosan (TMC)-based nanoparticles.

In one example, the disclosed nanoparticles are formulated as a particulate delivery system used for nasal administration. In one example the disclosed nanoparticles include liposomes, immune-stimulating complexes (ISCOMs) and/or polymeric particles, such as virosomes. In one example, the liposome is surface-modified (e.g., glycol chitosan or oligomannose coated). In one example, the liposome is fusogenic or cationic-fusogenic.

The disclosed immunogenic composition or nanoparticle-containing composition can also include one or more lipopeptides of bacterial origin, or their synthetic derivatives. Examples of lipid moieties include tri-palmitoyl-S-glyceryl cysteine (Pam3Cys), di-palmitoyl-S-glyceryl cysteine (Pam2Cys), single/multiple-chain palmitic acids and lipoamino acids (LAAs), and glycosylceramides that stimulate NKT cells, such as alpha-galactosylceramide.

In some examples, the disclosed immunogenic composition or nanoparticle-containing composition includes at least 10 μg, at least 20 μg, at least 50 μg, at least 80 μg, or at least 100 μg, such as 50 to 500 μg, 50 to 200 μg, 50 to 100 μg, such as 100 μg per dose of SARS-CoV-2 S protein, S1 protein or S2 protein (such as one having at least 90% sequence identity to SEQ ID NO: 1, 2, 3, 4, 5, 6, 9, 11, 12, 13, 14, 15, or 16). In some examples, a nanoparticle-containing composition includes at least 10 μg, at least 50 μg, at least 100 μg, at least 150 μg, or at least 200 g, such as 10 to 500 μg, 50 to 300 μg, 100 to 200 μg, such as 200 μg per dose of IL-15 (such as recombinant human IL-15, such as fragment Asn49-Ser162 of GenBank Accession No. NP_000576.1; SEQ ID NO: 7). In some examples, a nanoparticle-containing composition includes at least 10 μg, at least 50 μg, at least 100 μg, at least 150 μg, or at least 200 μg, such as 10 to 500 g, 50 to 300 μg, 100 to 200 μg, such as 200 μg per dose of D-type CpG oligodeoxynucleotide. In some examples, a nanoparticle-containing composition includes at least 100 μg, at least 250 μg, at least 500 μg, at least 800 μg, or at least 1 mg, such as 100 to 2000 μg, 500 to 2000 μg, 900 to 1000 g, such as 1 mg per dose of poly I:C. In some examples, a nanoparticle-containing composition includes at least 10 μl, at least 25 μl, at least 50 μl, at least 80 μg, or at least 100 μl, such as 10 to 200 μL, 50 to 200 μl, 90 to 110 μl such as 100 μl mg per dose of Adju-Phos® adjuvant (aluminum phosphate gel). In some examples, a nanoparticle-containing composition includes at least 0.05% w/w alum, such as at least 0.1% w/w, at least 0.2% w/w, at least 0.3% w/w, at least 0.4% w/w or at least 0.45% w/w alum, such as 0.05-2% w/w, 0.1-1% w/w, 0.4-0.6% w/w, or 0.45 to 0.55% w/w alum.

In some examples, a disclosed nanoparticle-containing composition includes (1) at least 10 g, at least 20 μg, at least 50 μg, at least 80 μg, or at least 100 μg, such as 50 to 500 μg, 50 to 200 g, 50 to 100 μg, such as 100 μg per dose of SARS-CoV-2 S protein, S1 protein, or S2 protein (such as one having at least 90% sequence identity to SEQ ID NO: 1, 2, 3, 4, 5, 6, 9, 11, 12, 13, 14, 15, or 16); (2) at least 10 μg, at least 50 μg, at least 100 μg, at least 150 μg, or at least 200 μg, such as 10 to 500 μg, 50 to 300 μg, 100 to 200 μg, such as 200 μg per dose of IL-15 (such as recombinant human IL-15 or fragment thereof, such as SEQ ID NO: 7); (3) at least 10 μg, at least 50 μg, at least 100 μg, at least 150 μg, or at least 200 μg, such as 10 to 500 μg, 50 to 300 μg, 100 to 200 μg, such as 200 μg per dose of D-type CpG oligodeoxynucleotide; (4) at least 100 μg, at least 250 μg, at least 500 μg, at least 800 μg, or at least 1 mg, such as 100 to 2000 μg, 500 to 2000 μg, 900 to 1000 μg, such as 1 mg per dose of poly I:C; and (5) at least 10 μl, at least 25 μl, at least 50 l, at least 80 μg, or at least 100 μl, such as 10 to 200 μL, 50 to 200 μl, 90 to 110 μl such as 100 μl mg per dose of Adju-Phos® adjuvant (aluminum phosphate gel).

In some examples, a disclosed nanoparticle-containing composition includes (1) at least 10 g, at least 20 μg, at least 50 μg, at least 80 μg, or at least 100 μg, such as 50 to 500 μg, 50 to 200 g, 50 to 100 μg, such as 100 μg per dose of SARS-CoV-2 S1 protein (such as one having at least 90% sequence identity to SEQ ID NO: 2, 3, 4, 5, 6, 12 or 15); (2) at least 10 μg, at least 50 μg, at least 100 μg, at least 150 μg, or at least 200 μg, such as 10 to 500 μg, 50 to 300 μg, 100 to 200 μg, such as 200 μg per dose of IL-15 (such as recombinant human IL-15 or fragment thereof, such as SEQ ID NO: 7); (3) at least 10 μg, at least 50 μg, at least 100 μg, at least 150 μg, or at least 200 μg, such as 10 to 500 μg, 50 to 300 μg, 100 to 200 μg, such as 200 μg per dose of D-type CpG oligodeoxynucleotide; (4) at least 100 μg, at least 250 μg, at least 500 μg, at least 800 μg, or at least 1 mg, such as 100 to 2000 μg, 500 to 2000 μg, 900 to 1000 μg, such as 1 mg per dose of poly I:C; and (5) at least 0.05% w/w alum, such as at least 0.1% w/w, at least 0.2% w/w, at least 0.3% w/w, at least 0.4% w/w or at least 0.45% w/w alum, such as 0.05-2% w/w, 0.1-1% w/w, 0.4-0.6% w/w, or 0.45 to 0.55% w/w alum.

### A. SARS-CoV-2 Spike Proteins

An exemplary sequence of native SARS-CoV-2 S protein (including the signal sequence, native ectodomain and TM and CT domains) is provided as SEQ ID NO: 1. The amino acid numbering used herein for residues of the SARS-CoV-2 S protein is with reference to the SARS-CoV-2 S sequence provided as SEQ ID NO: 1. With reference to the SARS-CoV-2 S protein sequence provided as SEQ ID NO: 1, the ectodomain of the SARS-CoV-2S protein includes about residues 16-1208. Residues 1-15 are the signal peptide, which is removed during cellular processing. The S1/S2 cleavage site is located at position 685/686. The HR1 is located at about residues 915-983. The central helix is located at about residues 988-1029. The HR2 is located at about 1162-1194. The C-terminal end of the S2 ectodomain is located at about residue 1208. In some embodiments, the protomers of the prefusion-stabilized SARS-CoV-2 S ectodomain trimer can have a C-terminal residue of the C-terminal residue of the HR2 (e.g., position 1194), or the ectodomain (e.g., position 1208), or from one of positions 1194-1208. The position numbering of the S protein may vary between SARS-CoV-2 stains, but the sequences can be aligned to determine relevant structural domains and cleavage sites.

The compositions and nanoparticles provided herein can include a SARS-CoV-2 S1 protein. An exemplary SARS-CoV-2 S1 protein is provided in SEQ ID NO: 2. Recently identified variants of SARS-CoV-2 S1 protein are provided in SEQ ID NOS: 3, 4, 5, 12 and 15 and can also be used.

In one example, an immunogenic composition or nanoparticle includes a SARS-CoV-2 S1 protein having at least 80%, least 85%, at least 90%, at least 95%, at least 96%, at least 97%, at least 98%, at least 99%, or 100% sequence identity to SEQ ID NO: 2, 3, 4, 5, 6, 12 or 15. In one example, an immunogenic composition or nanoparticle includes a SARS-CoV-2 S1 protein having at least 80%, least 85%, at least 90%, at least 95%, at least 96%, at least 97%, at least 98%, at least 99%, or 100% sequence identity to SEQ ID NO: 2. In one example, an immunogenic composition or nanoparticle includes a SARS-CoV-2 S1 protein having at least 90% sequence identity to SEQ ID NO: 2. In one example, an immunogenic composition or nanoparticle includes a SARS-CoV-2 S1 protein having at least 95% sequence identity to SEQ ID NO: 2. In one example, an immunogenic composition or nanoparticle includes a SARS-CoV-2 S1 protein having at least 90% sequence identity to SEQ ID NO: 3. In one example, an immunogenic composition or nanoparticle includes a SARS-CoV-2 S1 protein having at least 95% sequence identity to SEQ ID NO: 3. In one example, an immunogenic composition or nanoparticle includes a SARS-CoV-2 S1 protein having at least 90% sequence identity to SEQ ID NO: 4. In one example, an immunogenic composition or nanoparticle includes a SARS-CoV-2 S1 protein having at least 95% sequence identity to SEQ ID NO: 4. In one example, an immunogenic composition or nanoparticle includes a SARS-CoV-2 S1 protein having at least 90% sequence identity to SEQ ID NO: 5. In one example, an immunogenic composition or nanoparticle includes a SARS-CoV-2 S1 protein having at least 95% sequence identity to SEQ ID NO: 5. In one example, an immunogenic composition or nanoparticle includes a SARS-CoV-2 S1 protein having at least 90% sequence identity to SEQ ID NO: 6. In one example, an immunogenic composition or nanoparticle includes a SARS-CoV-2 S1 protein having at least 95% sequence identity to SEQ ID NO: 6. In one example, an immunogenic composition or nanoparticle includes a SARS-CoV-2 S1 protein having at least 90% sequence identity to SEQ ID NO: 12. In one example, an immunogenic composition or nanoparticle includes a SARS-CoV-2 S1 protein having at least 95% sequence identity to SEQ ID NO: 12. In one example, an immunogenic composition or nanoparticle includes a SARS-CoV-2 S1 protein having at least 90% sequence identity to SEQ ID NO: 15. In one example, an immunogenic composition or nanoparticle includes a SARS-CoV-2 S1 protein having at least 95% sequence identity to SEQ ID NO: 15.

In some examples, a variant SARS-CoV-2 S1 in the immunogenic composition or nanoparticle retains the mutation provided herein. For example, a SARS-CoV-2 S1 protein having at least 80%, least 85%, at least 90%, at least 95%, at least 96%, at least 97%, at least 98%, at least 99% sequence identity to SEQ ID NO: 3 can retain the N501Y substitution, 69-70del, and P681H substitution. For example, a SARS-CoV-2 S1 protein having at least 80%, least 85%, at least 90%, at least 95%, at least 96%, at least 97%, at least 98%, at least 99% sequence identity to SEQ ID NO: 4 can retain the L18F, D80A, D215G, R246I, K417N, E484K, N501Y, and D614G substitutions. For example, a SARS-CoV-2 S1 protein having at least 80%, least 85%, at least 90%, at least 95%, at least 96%, at least 97%, at least 98%, at least 99% sequence identity to SEQ ID NO: 5 can retain the L18F, T20N, P26S, D138Y, R190S, K417T, E484K, N501Y, D614G and H655Y substitutions. For example, a SARS-CoV-2 S1 protein having at least 80%, least 85%, at least 90%, at least 95%, at least 96%, at least 97%, at least 98%, at least 99% sequence identity to SEQ ID NO: 6 can retain the E484K and N501Y substitutions. For example, a SARS-CoV-2 S1 protein having at least 80%, least 85%, at least 90%, at least 95%, at least 96%, at least 97%, at least 98%, at least 99% sequence identity to SEQ ID NO: 12 can retain the A67V, Δ69-70, T95I, G142D, Δ143-145, Δ211, L121, ins214EPE, G339D, S371L, S373P, S375F, K417N, N440K., G446S, S477N. T478K, E484A or E484K, Q493R, G496S. Q498R, N501Y, Y505H, T547K. D614G, H655Y, N679K, and P681H mutations. For example, a SARS-CoV-2 S1 protein having at least 80%, least 85%, at least 90%, at least 95%, at least 96%, at least 97%, at least 98%, at least 99% sequence identity to SEQ ID NO: 15 can retain the T19R, G142D, E156G, 157-158 deletion, L452R, T478K, D614G, and P681R mutations. In some examples, the SARS-CoV-2 S1 protein has at least 80%, least 85%, at least 90%, or at least 95% sequence identity to SEQ ID NO: 2, and includes the following mutations: A67V, Δ69-70, T95I, G142D, Δ143-145, Δ211, L212T, ins214EPE, G339D, S371L, S373P, S375F, K417N, N440K, G446S, S477N, T478K, E484A or E484K, Q493R, G496S, Q498R, N501Y, Y505H, T547K, D614G, H655Y, N679K, and P681H (numbering based on SEQ ID NO: 1).

The compositions and nanoparticles provided herein can include a SARS-CoV-2 S2 protein. An exemplary SARS-CoV-2 S2 protein is provided in SEQ ID NO: 9. In some examples, the SARS-CoV-2 S2 protein includes K986P and V987P substitutions (numbering based on SEQ ID NO: 1). In some examples, the SARS-CoV-2 S2 protein has at least 80%, least 85%, at least 90%, at least 95%, at least 96%, at least 97%, at least 98%, or at least 99% sequence identity to SEQ ID NO: 9, and includes K986P and V987P substitutions (numbering based on SEQ ID NO: 1). In some examples, the SARS-CoV-2 S2 protein has at least 80%, least 85%, at least 90%, or at least 95% sequence identity to SEQ ID NO: 9, and includes N764K, D796Y, N856K, Q954H, N969K, and L981F substitutions (numbering based on SEQ ID NO: 1). Additional exemplary SARS-CoV-2 S2 proteins are provided in SEQ ID NOs: 13 and 16. In some examples, the SARS-CoV-2 S2 protein has at least 80%, least 85%, at least 90%, or at least 95% sequence identity to SEQ ID NO: 13 or 16 and includes a D950N mutation.

The compositions and nanoparticles provided herein can include a SARS-CoV-2 S protein. Exemplary SARS-CoV-2 S proteins are provided in SEQ ID NOS: 1, 11 and 14. In some examples, the SARS-CoV-2 S protein includes K986P and V987P substitutions (numbering based on SEQ ID NO: 1). In some examples, the SARS-CoV-2 S protein does not include the signal peptide (aa 1-15 of SEQ ID NO. 1, 11 and 14). In some examples, the SARS-CoV-2 S protein has at least 80%, least 85%, at least 90%, at least 95%, at least 96%, at least 97%, at least 98%, or at least 99% sequence identity to SEQ ID NO: 1 (or aa 16-1208 of SEQ ID NO: 1), and includes K986P and V987P substitutions (numbering based on SEQ ID NO: 1). In some examples, the SARS-CoV-2 S protein has at least 80%, least 85%, at least 90%, at least 95%, at least 96%, at least 97%, or at least 98% sequence identity to SEQ ID NO: 1 (or aa 16-1208 of SEQ ID NO: 1), and includes the following mutations: A67V, Δ69-70, T95I, G142D, Δ143-145, Δ211, L212I, ins214EPE, G339D, S371L, S373P, S375F, K417N, N440K, G446S, S477N, T478K, E484A or E484K, Q493R, G496S, Q498R, N501Y, Y505H, T547K, D614G, H655Y, N679K, P681H, N764K, D796Y, N856K, Q954H, N969K, L981F (numbering based on SEQ ID NO: 1). In some examples, the SARS-CoV-2 S protein has at least 80%, least 85%, at least 90%, at least 95%, at least 96%, at least 97%, or at least 98% sequence identity to SEQ ID NO: 11 (or aa 16-1205 of SEQ ID NO: 11) and includes the following mutations: A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F. In some examples, the SARS-CoV-2 S protein has at least 80%, least 85%, at least 90%, at least 95%, at least 96%, at least 97%, or at least 98% sequence identity to SEQ ID NO: 14 (or aa 16-1206 of SEQ ID NO: 14) and includes the following mutations: T19R, G142D, E156G, 157-158 deletion, L452R, T478K, D614G, P681R, D950N (numbering based on SEQ ID NO: 1).

The compositions and nanoparticles provided herein can include a SARS-CoV-2 S protein, S1 protein, or S2 protein having the mutations found in the omicron variant, namely: A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F (numbering based on SEQ ID NO: 1).

Thus, in some examples, the SARS-CoV-2 S protein, S1 protein, or S2 protein present in a disclosed immunogenic composition or nanoparticle is a variant SARS-CoV-2 S protein, S1 protein, or S2 protein. Such variants can include insertions, substitutions, or deletions of one or more amino acid residues, glycosylation variants, organic and inorganic salts, covalently modified derivatives of a native Wuhan S protein, S1 protein, or S2 protein, or a precursor thereof. Such variants may maintain one or more of the functional, biological activities of a SARS-CoV-2 S protein, S1 protein, or S2 protein, such as binding to cell surface receptor.

Thus, in some examples, the SARS-CoV-2 S protein, S1 protein, or S2 protein present in a disclosed immunogenic composition or nanoparticle is isolated or purified, for example substantially free of other proteins and nucleic acid molecules.

One skilled in the art will appreciate that as an alternative to using a SARS-CoV-2 S protein, S1 protein, or S2 protein in the disclosed immunogenic compositions and nanoparticles, a coding sequence can instead be used, such as an mRNA molecule encoding any SARS-CoV-2 S protein, S1 protein, or S2 protein provided herein.

### B. Exemplary Adjuvants

The disclosed immunogenic compositions include one or more adjuvants. In one example, the one or more adjuvants includes aluminum (alum), such as aluminum sulfate, aluminum potassium sulfate, aluminum hydroxide, or aluminum phosphate. In one example, the one or more adjuvants includes an aluminum gel (e.g., alhydrogel). In one example, the one or more adjuvants includes phosphate.

The disclosed nanoparticles include one or more adjuvants. In one example, the one or more adjuvants includes one or more toll-like receptors (TLR) agonists, such as an agonist of TLR9 (e.g., CpG oligonucleotides (such as D-type CpG oligodeoxynucleotides)) and/or an agonist of TLR3 (e.g., polyinosine-polycytidic acid (poly(I:C)) or polyIC12 U or poly-ICLC). In one example, the one or more adjuvants includes IL15 (such as recombinant human IL-15 or fragment thereof, such as SEQ ID NO: 7, or a superagonist IL-15, or an IL-15/IL-15Ralpha heterodimer). In one example, the one or more adjuvants include IL15 (such as recombinant human IL-15 or fragment thereof, such as SEQ ID NO: 7) and one or more TLR agonists. In one example, the one or more adjuvants include IL15 (such as recombinant human IL-15 or fragment thereof, such as SEQ ID NO: 7), CpG oligonucleotides (such as D-type CpG oligodeoxynucleotides) and poly(I:C).

The disclosed immunogenic compositions and nanoparticles in some examples further include other adjuvants, such as lipid-A and derivatives or variants thereof, oil-emulsions, saponins, neutral liposomes, liposomes containing the vaccine and cytokines, non-ionic block copolymers (e.g., those containing polyoxyethylene (POE) and polyoxypropylene (POP), such as POE-POP-POE block copolymers), chemokines, IL-12, Flt3 ligand, monophosphoryl lipid A (MLA) (such as a clinical grade MLA formulation, such as MPL® (3-O-desacyl-4′-monophosphoryl lipid A) among many other exemplary adjuvants, may be used as an adjuvant (Newman et al., 1998, Critical Reviews in Therapeutic Drug Carrier Systems 15:89-142). Adjuvants can help to stimulate the immune system in a non-specific way, thus enhancing the immune response to a SARS-CoV-2 S1 protein.

### C. Exemplary Nanoparticles

The nanoparticles provided herein and used in the booster vaccine can be composed of particles, such as those that are about 1 to 300 nanometres (nm) in diameter, such as about 50 to 300 nm, 50 to 100 nm, 50 to 200 nm, 70 to 100 nm, or 70 to 200 nm in diameter. The nanoparticles can be designed to be similar in size to a SARS-CoV-2 virus. The nanoparticles of the booster vaccine include (1) antigen, such as a SARS-CoV-2 S protein, S1 subunit, or S2 subunit (or mRNA encoding such) and (2) other reagents, such as one or more adjuvants. As described above, the SARS-CoV-2 protein can have at least 80%, least 85%, at least 90%, at least 95%, at least 96%, at least 97%, at least 98%, at least 99%, or 100% sequence identity to SEQ ID NO: 1, 2, 3, 4, 5, 6, 9 or aa 16-1208 of SEQ ID NO: 1. Exemplary adjuvants that can be included in the nanoparticles are described above, such as IL15 (such as recombinant human IL-15 or fragment thereof, such as SEQ ID NO: 7) and one or more TLR agonists. In one example, the one or more adjuvants include IL15 (such as recombinant human IL-15 or fragment thereof, such as SEQ ID NO: 7), CpG oligonucleotides (such as D-type CpG oligodeoxynucleotides) and poly(I:C).

Nanoparticle (NP) scaffold platforms that can be used include inorganic NPs, polymeric NPs, liposomes, virus-like particles (VLPs), self-assembling NPs, and dendrimers.

In some examples, a nanoparticle is polymeric, such as a nanocapsule or nanosphere. Biodegradable polymers can be natural or synthetic nonimmunogenic monomers, and having low cytotoxicity. Exemplary polymers that can be used include chitosan, PLGA, polyethylene glycol (PEG), polycaprolactone, and dextran. In some examples, nanoparticles are composed of PLGA, poly(F-caprolactone) (PCL), PEG, poly(lactic acid) (PLA) or combinations thereof. In some examples, nanoparticles are composed of lipids (LNPs), such as 1,2-dilinoleyloxy-3-dimethylaminopropane (DOPE), 1,2-dioleoyl-sn-glycero-3-phosphoethanolamine (DOTAP), 1,2-Dioleyl-3-trimethylammonium-propane chloride salt (DSPC), 1,2-Distearoyl-sn-glycero-3-phosphocholine; dipalmitoylphosphatidylcholine (PC), phosphatidylserine (PS), cholesterol, or combinations thereof.

The disclosed nanoparticles can be made by any suitable method. Methods of making nanoparticles is known (e.g., see Butkovich et al., Wiley Interdiscip Rev Nanomed Nanobiotechnol, 2020 Nov. 8; e1681 and Akagi et al., Biomaterials 32:4959-496, both herein incorporated by reference in their entireties).

### Methods of Eliciting an Immune Response

The disclosed immunogenic compositions and nanoparticles can be administered to a subject to induce an immune response to SARS-CoV-2 S protein, S1 protein and/or S2 protein in the subject. In a particular example, the subject is a human. The immune response can be a protective immune response, for example a response that inhibits subsequent infection with SARS-CoV-2. Elicitation of the immune response can also be used to treat or inhibit SARS-CoV-2 infection and illnesses associated with the SARS-CoV-2 infection.

Provided herein are methods of eliciting an immune response against SARS-CoV-2 in a subject. In one example, such a method includes (1) administering to the subject an effective amount of a primary dose of an immunogenic composition provided herein (e.g., one containing a SARS-CoV-2 S protein, S1 protein, or S2 protein, such as one having at least 80%, least 85%, at least 90%, at least 95%, at least 96%, at least 97%, at least 98%, at least 99%, or 100% sequence identity to SEQ ID NO: 1, 2, 3, 4, 5, 6, 9, 11, 12, 13, 14, 15, 16, aa 16-1208 of SEQ ID NO: 1, aa 16-1205 of SEQ ID NO: 11, or aa 16-1206 of SEQ ID NO: 14 [or mRNA encoding such], and one or more adjuvants, such as alum) and (2) subsequently administering to the subject an effective amount of one or more booster doses of the nanoparticles provided herein (e.g., one containing a SARS-CoV-2 S protein, S1 protein, or S2 protein, such as one having at least 80%, least 85%, at least 90%, at least 95%, at least 96%, at least 97%, at least 98%, at least 99%, or 100% sequence identity to SEQ ID NO: 1, 2, 3, 4, 5, 6, 9, 11, 12, 13, 14, 15, 16, aa 16-1208 of SEQ ID NO: 1, aa 16-1205 of SEQ ID NO: 11, or aa 16-1206 of SEQ ID NO: 14 [or mRNA encoding such] and one or more adjuvants, such an agonist of toll-like receptor 3, an agonist of toll-like receptor 9, IL-15, or combinations thereof, such as CpG, Poly I:C and IL-15), thereby eliciting the immune response. In some examples, the primary dose is administered IM, and the one or more booster doses are administered IN.

The nanoparticles provided herein can be used in combination with other SARS-CoV-2 vaccines, such as a nucleic acid vaccine (e.g., DNA or mRNA vaccines), to elicit an immune response against SARS-CoV-2 in a subject. In such examples, the nanoparticles are provided IN in one or more subsequent booster doses. For example, such a method can include (1) administering to the subject an effective amount of a primary dose of a composition that includes mRNA encoding a SARS-CoV-2 S protein, S1 protein, or S2 protein, such as one having at least 80%, least 85%, at least 90%, at least 95%, at least 96%, at least 97%, at least 98%, at least 99%, or 100% sequence identity to SEQ ID NO: 1, 2, 3, 4, 5, 6, 9, 11, 12, 13, 14, 15, 16, aa 16-1208 of SEQ ID NO: 1, aa 16-1205 of SEQ ID NO: 11, or aa 16-1206 of SEQ ID NO: 14 (such as an mRNA having at least 80%, least 85%, at least 90%, at least 95%, at least 96%, at least 97%, at least 98%, at least 99%, or 100% sequence identity to nt 46-2005 of SEQ ID NO: 8, an mRNA having at least 80%, least 85%, at least 90%, at least 95%, at least 96%, at least 97%, at least 98%, at least 99%, or 100% sequence identity to nt 2059 to 3624 of SEQ ID NO: 8, an mRNA having at least 80%, least 85%, at least 90%, at least 95%, at least 96%, at least 97%, at least 98%, at least 99%, or 100% sequence identity to nt 46-3624 of SEQ ID NO: 8, or an mRNA having at least 80%, least 85%, at least 90%, at least 95%, at least 96%, at least 97%, at least 98%, at least 99%, or 100% sequence identity to SEQ ID NO: 8), and one or more adjuvants and (2) subsequently administering to the subject an effective amount of one or more booster doses of the nanoparticles provided herein (e.g., one containing a SARS-CoV-2 S protein, S1 protein, or S2 protein, such as one having at least 80%, least 85%, at least 90%, at least 95%, at least 96%, at least 97%, at least 98%, at least 99%, or 100% sequence identity to SEQ ID NO: 1, 2, 3, 4, 5, 6, 9, 11, 12, 13, 14, 15, 16, aa 16-1208 of SEQ ID NO: 1, aa 16-1205 of SEQ ID NO: 11, or aa 16-1206 of SEQ ID NO: 14 [or mRNA encoding such] and one or more adjuvants, such an agonist of toll-like receptor 3, an agonist of toll-like receptor 9, IL-15, or combinations thereof [such as CpG, Poly I:C and IL-15]), thereby eliciting the immune response. In some examples, the primary dose is an mRNA vaccine, such as Pfizer-BioNTech's BNT162b2 vaccine, or Moderna's mRNA-1273 vaccine. In some examples, the primary dose is AstraZeneca's chimpanzee adenovirus-vectored, ChAdOx1 nCoV-19 vaccine (AZC1222) expressing spike protein. In one example, the primary dose is a protein vaccine, such as Novavax's NVX-CoV2373 vaccine, which contains a full-length, prefusion spike protein. In one example, the primary dose is an adenovirus serotype 26 vectored vaccine, such as Johnson & Johnson's JNJ-78436735 vaccine (also known as Ad26.COV2.S). In one example, the primary dose is a DNA vaccine, such as Inovio's INO-4800 vaccine encoding spike protein. In some examples, the primary dose is administered IM, and the one or more booster doses are administered IN.

A subject can be selected for treatment that has or is at risk for developing SARS-CoV-2 infection, for example because of exposure or the possibility of exposure to the SARS-CoV-2. Following administration of a disclosed immunogenic compositions and/or nanoparticles, the subject can be monitored for infection or symptoms associated with SARS-CoV-2 infection.

Typical subjects intended for treatment with the therapeutics and methods of the present disclosure include humans, as well as non-human primates and other animals, such as hamsters, ferrets, lions, tigers, bats, rabbits, bank voles, mink, pigs, deer, hyenas, otters, cats and dogs. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize coronavirus infection. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The administration of a disclosed immunogenic composition and/or nanoparticles (immunogen) can be for prophylactic or therapeutic purpose. When provided prophylactically, the immunogen is provided in advance of any symptom, for example, in advance of infection. The prophylactic administration of the immunogen serves to prevent or ameliorate any subsequent infection. When provided therapeutically, the immunogen is provided at or after the onset of a symptom of infection, for example, after development of a symptom of SARS-CoV-2 infection or after diagnosis with the SARS-CoV-2 infection. The immunogen can thus be provided prior to the anticipated exposure to the SARS-CoV-2 so as to attenuate the anticipated severity, duration or extent of an infection and/or associated disease symptoms, after exposure or suspected exposure to the SARS-CoV-2, or after the actual initiation of an infection. The mucosal vaccine can also be used to reduce the risk of transmission to other individuals by reducing the viral load in the nasal and upper respiratory mucosa.

The disclosed immunogenic compositions and/or nanoparticles, are provided to a subject in an amount effective to induce or enhance an immune response against the SARS-CoV-2 S protein in the subject, such as a human. The actual dosage of disclosed immunogen in the disclosed immunogenic compositions and/or nanoparticles can vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

The amount of antigen utilized in a disclosed immunogenic composition and/or nanoparticle composition can be selected based on the subject population (e.g., infant or elderly). An optimal amount for a particular composition can be ascertained by standard studies involving observation of antibody titers and other responses in subjects. It is understood that an effective amount of immunogen can include an amount that is ineffective at eliciting an immune response by administration of a single dose, but that is effective upon administration of multiple dosages, for example in a prime-boost administration protocol.

The disclosed immunogenic compositions and nanoparticles can be used in coordinate (or prime-boost) vaccination protocols or combinatorial formulations. In certain embodiments, the disclosed immunogenic compositions and nanoparticles and coordinate immunization protocols employ separate immunogens or formulations, each directed toward eliciting an anti-viral immune response, such as an immune response to SARS-CoV-2 S protein. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

The one or more nanoparticle boosts can be administered IN, such as one boost, two boosts, three boosts, four boosts, five boosts, six boosts or more (such as 1, 2, 3, 4, 5, 6, 7, 8, 9, 10, 11, 12, 13, 14, 15, 16, 17, 18, 19 or 20 booster doses, such as 1-20, 2-20, 3-20, 5-10, or 3-20 booster doses) can be administered to a subject over days, weeks months, or years. Different dosages can be used in a series of sequential immunizations. For example a boost may include a smaller dose of immunogen than the primer dose.

In some examples, the first booster dose is administered at least 2 weeks, at least 3 weeks, at least 4 weeks, at least 5 weeks, at least 6 weeks, at least 7 weeks, at least 8 weeks, at least 10 weeks, at least 12 weeks, or at least 16 weeks after administering the prime dose. In some embodiments, a nanoparticle boost can be administered about two, about three to eight, or about four, weeks following the prime, or about several months after the prime. In some embodiments, the boost can be administered about 5 months, about 6 months, about 7 months, about 8 months, about 10 months, about 12 months, about 18 months, or about 24 months, after the prime, or more or less time after the prime. In some examples, the first booster dose is administered a year or more after administering the prime dose. Periodic additional boosts can also be used at appropriate time points to enhance the subject's “immune memory.” For example, additional boosts can be administered about 3, about 4, about 5, about 6, about 7, about 8, about 10, about 12, about 18, or about 24 months after the first boost. In one example, additional boosts can be administered about every 3-24 months after the first boost, such as every 3, 4, 5, 6, 7, 8, 9, 10, 11, 12, 13, 14, 15, 16, 17, 18, 19, 20, 21, 22, 23, or 24 months. The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

Upon administration of a disclosed immunogenic composition and/or nanoparticle booster(s), the immune system of the subject typically responds by producing antibodies specific for the SARS-CoV-S1 protein, S2 protein, or S protein included in [or encoded by] the immunogen. Such a response signifies that an immunologically effective dose was delivered to the subject.

In some embodiments, the antibody response of a subject will be determined in the context of evaluating effective dosages/immunization protocols. In most instances it will be sufficient to assess the antibody titer in serum or plasma obtained from the subject. Decisions as to whether to administer additional nanoparticle booster inoculations and/or to change the amount of the therapeutic agent administered to the individual can be at least partially based on the antibody titer level. The antibody titer level can be based on, for example, an immunobinding assay which measures the concentration of antibodies in the serum which bind to an antigen including, for example, the recombinant SARS-CoV-2 S ectodomain trimer included in the immunogen.

SARS-CoV-2 infection does not need to be completely eliminated or reduced or prevented for the methods to be effective. For example, elicitation of an immune response to SARS-CoV-2 with a disclosed immunogenic composition and/or nanoparticle booster(s) can reduce or inhibit SARS-CoV-2 infection by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to SARS-CoV-2 infection in the absence of the immunogen. In additional examples, SARS-CoV-2 replication can be reduced or inhibited by the disclosed methods. SARS-CoV-2 replication does not need to be completely eliminated for the method to be effective. For example, the immune response elicited using a disclosed immunogenic composition and/or nanoparticle booster(s) can reduce SARS-CoV-2 replication by a desired amount, for example, by at least 10%, at least 20%, at least 50%, at least 60%, at least 70%, at least 80%, at least 90%, at least 95%, at least 98%, or even at least 100% (elimination or prevention of detectable SARS-CoV-2 replication, as compared to SARS-CoV-2 replication in the absence of the immune response.

In some examples, the disclosed methods inhibit or prevent severe COVID19 disease in the subject. In some examples, the disclosed methods inhibit or reduce as one or more signs or symptoms of COVID-19 (for example, as measured by diagnostic assays, such as imaging, blood tests, pulse oximetry, and the like). The signs or symptoms of COVID-19 do not need to be completely eliminated or reduced or prevented for the method to be effective. For example, administration of a therapeutically effective amount of a disclosed immunogenic composition and/or nanoparticle booster(s) can decrease one or more signs or symptoms of COVID-19 (such as one or more of headache, loss of smell, loss of taste, nasal congestion, rhinorrhea, cough, muscle pain, sore throat, fever, fatigue, breathlessness, muscle weakness, cognitive issues, breathing difficulties, mild pneumonia, pneumonia, dyspnea, hypoxia, respiratory failure, septic shock, blood clots, cytokine storm, lung fibrosis, multiorgan dysfunction and acute respiratory distress syndrome (ARDS)) by at least 10%, at least 20%, at least 50%, at least 60%, at least 70%, at least 80%, at least 90%, at least 95%, at least 98%, or even at least 100% (complete treatment of COVID-19), as compared to a suitable control (such as absence of immunogen).

In some embodiments, administration of a therapeutically effective amount of a disclosed immunogenic composition and/or nanoparticle booster(s) to a subject induces a neutralizing immune response in the subject. To assess neutralization activity, following immunization of a subject, serum can be collected from the subject at appropriate time points, frozen, and stored for neutralization testing. Methods to assay for neutralization activity include, but are not limited to, plaque reduction neutralization (PRNT) assays, microneutralization assays, flow cytometry based assays, single-cycle infection assays. In some embodiments, the serum neutralization activity can be assayed using a SARS-CoV-2 pseudovirus, similar to that used for SARS-CoV (Martin et al., Vaccine 26, 6338, 2008; Yang et al., Nature 428, 561, 2004; Naldini et al., PNAS 93, 11382, 1996; Yang et al., PNAS 102, 797, 2005). In some examples, the method induces a neutralizing antibody titer of at least 100, at least 200, at least 300, at least 350, such as 100 to 400, 200 to 375, such as 374.

In some embodiments, administration of a therapeutically effective amount of a disclosed immunogenic composition and/or nanoparticle booster(s) to a subject provides sterilizing protection against SARS-CoV-2 to the subject, for example in the upper and lower respiratory tracts. To assess sterilizing protection, following immunization of a subject, viral replication (sgRNA) can be measure in sample obtained from a subject (e.g., nasal swab, buccal swab, saliva, or blood sample).

In some embodiments, administration of a therapeutically effective amount of a disclosed immunogenic composition and/or nanoparticle booster(s) to a subject increases dimeric IgA production by the subject (for example in BAL), such as an increase of at least 20%, at least 50%, at least 90%, at least 100%, at least 200%, at least 300%, at least 400%, or even at least 500% as compared to no immunogen or as compared to other SARS-CoV-2 vaccine boosters that do not include the disclosed nanoparticles.

In some embodiments, administration of a therapeutically effective amount of a disclosed immunogenic composition and/or nanoparticle booster(s) to a subject increases IFN-α production by the subject (for example as detected in BAL supernatants), such as an increase of at least 20%, at least 50%, at least 90%, at least 100%, at least 150% or at least 200% as compared to no immunogen or as compared to other SARS-CoV-2 vaccine boosters that do not include the disclosed nanoparticles.

In some examples, the disclosed methods further include administering to the subject a COVID-19 treatment, such as one or more of remdesivir, galidesivir, lenzilumab, molnupiravir, hydroxychloroquine, dexamethasone, arbidol, favipiravir, baricitinib, lopinavir/ritonavir, zinc ions, and interferon beta-1b.

The following examples are provided to illustrate certain particular features and/or embodiments. These examples should not be construed to limit the disclosure to the particular features or embodiments described.

### Example 1

### Materials and Methods

This example provides the materials and methods used for the results described in Examples 2-6.

Animals. 18 Indian-origin adult male rhesus macaques (Macaca mulatta), 3-8 years old, were included in the study. At the start of the study, all animals were free of cercopithecine herpesvirus 1, SIV, simian type-D retrovirus, and simian T lymphotropic virus type 1.

Study design for subunit vaccine with adjuvants. No animals had been exposed to SARS-CoV-2 prior to challenge, and all tested negative for SARS-CoV-2 before the study. Six macaques were included in the naïve group, five of them had previously gone through HIV glycopeptide vaccination, and one of them (DFKL) had been exposed to 8 repeated challenges of SIVmac251, but never showed any viral loads for SIV. An additional 12 macaques that were never enrolled in any other studies were divided into 2 vaccine groups. Group 1 (n=6, Alum group) was given systemic vaccine primed at Week 0 and boosted at Week 3 and Week 16 with SARS-CoV-2 S1 protein with alum adjuvant. All the vaccinations were given intramuscularly (IM) in group 1. Group 2 (n=6, CP15 group) was administered with a mucosal vaccine primed at Week 0 with S1 protein with alum adjuvant, and boosted at Week 3, 6, and 16 with S1 protein with CP15 adjuvant (administered IM), which was a combination of CpG (SEQ ID NO: 10)+poly I:C (polyinosinic-polycytidylic acid; average size: 1.5-8 kb)+IL-15 in DOTAP or PLGA. For immunization, each vaccine contained 100 μg of recombinant SARS-CoV-2 (2019-nCoV) Spike S1 protein (Cat: 40591-V08H, Sino Biological, endotoxin level: <0.001 U/μg; Val 16-Arg685 of SEQ ID NO: 1). 100 μl of Adju-Phos® adjuvant (Aluminum phosphate gel, InvivoGen) was used as adjuvant. CP15 adjuvant was a combination of 200 μg per dose of D-type CpG oligodeoxynucleotide, 1 mg per dose of PolyI:C (InvivoGen), and 200 μg per dose of recombinant human IL-15 (Sino Biological). The mucosal vaccine incorporated S1 protein with CP15, formulated in nanoparticles either in PLGA (Alchem Laboratories) for the first 2 doses or in DOTAP (100 μl per dose; Roche) for the last dose. After vaccination, blood and BAL fluid samples were collected at the times noted and analyzed.

BAL sample collection. Animals were anesthetized, and then up to 10 mL/kg of sterile saline was instilled into the lungs. The instilled fluid (up to 90%) was recovered by suction. A 100 m cell strainer was used to remove large pieces from the collected BAL fluid. The cells were then washed with RIO medium (RPMI-1640 with 10% fetal bovine serum) and centrifuged. BAL fluid and cells were collected for analysis or cryopreservation.

ELISA assay to detect S1-specific antibody responses. The BAL samples collected from each individual monkey were concentrated roughly 30-fold using Amicon Ultra centrifugal filter units (10 kDa cutoff, Millipore Sigma). The total IgA quantity in the concentrated BAL samples was determined using the Monkey IgA ELISA development kit (HRP) (MabTech) following the manufacturer's protocol.

Total IgG quantities in the plasma and concentrated BAL samples were measured using the Rhesus Monkey IgG-UNLB (Southern Biotech) as the IgG standard. In brief, high-binding 96-well plates (Santa Cruz Biotechnology) were coated with serial dilutions of IgG standard and the samples in 1×PBS, pH 7.4 and incubated at 4° C. overnight. Afterward, the plates were washed three-times with wash buffer (0.05% Tween-20 in 1×PBS, pH 7.4) and blocked with 300 μL of 2% sodium casein in 1×PBS at 37° C. for 1 h. Following three washes, 100 μL of Goat anti-Monkey IgG (H+L) Secondary Antibody [HRP] (Novus Biologicals NB7215) was applied to each well with 1:20,000 dilutions in 1×PBS. The plates were incubated at room temperature for 30 minutes and then extensively washed with the wash buffer five times. Then, TMB 2-component microwell peroxidase substrates (SeraCare) were applied to the well plates following the manufacturer's instructions. The plates were developed in the dark at room temperature for 30 minutes and then quenched by adding 100 μL/well of 1 M H₃PO₄ solution. Absorbance was read using SpectraMax M5 Multi-Mode Microplate Reader (Molecular Devices) at 450 nm and 550 nm. The concentrations of IgA and IgG were determined using GraphPad Prism 8 software with sigmoidal nonlinear regression.

The antigen-specific binding assays were performed similarly but with 100 ng/well of the SARS-CoV-2 Spike S1-His Recombinant Protein (Sino Biological) as the coating antigen. After blocking the plates with 2% sodium casein, the concentrated BAL samples were applied in duplicate with a series of 2-fold dilutions starting from an IgA or IgG concentration of 2 μg/mL. In the case of antiserum analysis, plasma samples were serially diluted 2/4/5-fold starting from a 1:150 dilution and run in duplicate. The plates were incubated at room temperature for 1 hr., followed by four washes. Subsequent steps of incubation with HRP-labeled secondary antibody and TMB substrate were followed as described above. In case of BAL IgA binding assay, Goat Anti-Monkey IgA (alpha-chain specific)-HRP conjugate (1:5,000 dilutions, Alpha Diagnostic) was used as a secondary antibody. Post assay, area under the curve, endpoint titer, and EC₅₀ values were computed by GraphPad Prism 8 software with sigmoidal nonlinear regression.

ELISA assay to detect dimeric IgA in BAL. DuoSet ELISA Ancillary Reagent Kit 2 (R&D Systems) was used. Briefly, 100 ng/well of the SARS-CoV-2 spike S1 protein was coated and blocked as described above. Original BAL samples from vaccinated and naïve animals were added in duplicate to the plate and incubated at room temperature for 1 hr., followed by 5 washes. Mouse anti-rhesus J chain [CA1L_33e1_A1a3] antibody (1:1000 dilutions, NIH nonhuman primate reagent resource), and Goat anti-mouse IgG-HRP conjugate (1:10,000 dilutions, R&D Systems) were added and each followed by 1 hr. incubation at room temperature and five washes. Plate development and reading was performed as described above.

Plaque reduction neutralization test (PRNT). The PRNT was performed in duplicate using Vero E6 cells (ATCC, cat. no. CRL-1586), and 30 PFU challenge titers of SARS-CoV-2 virus (USA-WA1/2020 strain). Serum samples were tested at a starting dilution of 1:20 and were serially diluted 3-fold up to final dilution of 1:4860. After serum incubation with 30 PFU of SARS-CoV-2 virus for 1 hr. at 37° C., serial dilutions of virus-serum mixtures were added onto Vero E6 cell monolayers. Cell culture medium with 1% agarose was added to the cells, following incubation for 1 hr. at 37° C. with 5% CO₂. The plates were fixed and stained after three days of culture. Antibody titer IC50, and IC90 were defined as the highest serum dilution resulting in 50 and 90% reduction of plaques, respectively.

Intracellular cytokine staining assay. SARS-CoV-2-specific T cells were measured from mononuclear cells of the fresh or thawed cryopreserved BAL and PBMC samples by flow cytometric intracellular cytokine analysis, as previously described in detail^{16,41}. Briefly, cell samples were stimulated with 2 μg/ml of SARS-CoV-2 S1 protein (Sino Biological) for PBMC, and 5 μg/ml for BAL samples with 0.15 μg/ml of brefeldin A at 37° C. 5% CO₂ overnight. Negative controls received an equal concentration of brefeldin A (without protein). Cell activation cocktail with PMA (20.25 pM) and ionomycin (335 pM) and 0.15 μg/ml of brefeldin A (Biolegend) was added to the cells as positive control. For flow cytometric analysis, the BAL cells were centrifuged after a wash with 0.25% PBS, and then stained with viability dye (Invitrogen) and antibody mixtures. Antibodies: PE-Cy7-CD3, BV605-CD4, APC-Cy7-CD8, Alexa Fluor® 700-CD45 were from BD Biosciences, FITC-CD28, Pe-Cy5-CD95, BV711—TNFα, IFNγ-PE or -PerCP, Alexa Fluor® 647-IL4, BV785-IL2, BV421-IL-17A, BV785-CD14, BV421-CD16 were from Biolegend; PE-IL13 was from Miltenyi Biotech. After cell surface staining, eBioscience™ FOXP3/Transcription Factor Staining Buffer Set (ThermoFisher) was used for cell permeabilization, followed by intracellular staining. An LSRII flow cytometer with 4 lasers (BD Bioscience) and FlowJo software (Becton Dickinson) was used for data acquisition and analyses. For each animal, and each time point, the antigen-specific T cell responses were reported as the frequencies of cytokine-positive cells in the samples stimulated with S1 protein minus those in the medium-only control.

IFN-α ELISA and chemokine/cytokine Bioplex assay after poly I:C plus S1 protein stimulation of BAL samples. Cryopreserved BAL (from the one-week post second vaccination timepoint) were thawed and resuspended at a concentration of 3-4 million cells/ml in serum free medium AIM® (ThermoFisher). Poly I:C (2 g/ml) was added to the cells in the presence or absence of 2 μg/ml of SARS-CoV-2 spike S1 protein (Sino Biological, endotoxin level: <0.001 U/μg). After 18 hrs. of culture at 37° C., 5% CO₂, supernatant was collected and frozen at −20° C. for IFN-α ELISA, and Chemokine/Cytokine Bioplex Assay using an LSRII cytometer. LEGENDplex™ NHP Chemokine/Cytokine Panel (13-plex, Biolegend) was used to measure the following 13 chemokines and cytokines: TNF-α, IL-1β, IL-6, IL-8, MIP1-α, MIP1-β, RANTES, MCP-1, IFN-γ, MIG, IP-10, ITAC, and Eotaxin. Pan-IFN-α (including subtypes α1, 2, 4, 5, 6, 7, 8, 10, 14, 16 and 17) ELISA kit (Mabtech) was used to measure the total concentration of IFN-α. Both assays were performed in accordance with the manufacturers' instructions.

SARS-CoV-2 challenge. At week 20, 25 days after the last boost, all 18 animals were challenged with 1.5×10⁴ pfu SARS-CoV-2 virus (USA-WA1/2020 strain) which was equivalent to approximately 1.25×10⁵ TCID50 SARS-CoV-2 virus (USA-WA1/2020 strain), equivalent to or slightly greater than the challenge dose used in some earlier macaque challenge studies noted above. The challenge virus was obtained from BEI Resources (Lot #70038893) and has a reported infectious titer in Vero E6 cells of 3×10⁶ pfu/mL. The virus was diluted in PBS to the indicated challenge dose level. The virus was given intranasally and intratracheally, each route with 1 ml (0.5 ml for each nares) to make sure the virus was delivered to both upper and lower airway. Nasal swab and BAL fluid samples were collected on days 2 and 4 after challenge to measure the viral load.

Subgenomic RNA and viral RNA assay. SARS-CoV-2 RNA levels were monitored by RT-PCR by BIOQUAL, Inc. as described previously. Briefly, RNA was extracted from nasal swab and BAL fluid samples collected at the different time-points. After reverse transcription, cDNAs were run in duplicate to quantify subgenomic or viral RNA using different primer/probe sets, targeting the viral E gene mRNA or the viral nucleocapsid, respectively. The sequences of the primers/probes have been published previously^{5,42}. Viral loads are shown as copies per ml for BAL fluid and per swab for nasal samples with a cutoff value of 50 copies for each assay.

Lower respiratory histopathology and immunohistochemistry. Seven or ten days after SARS-CoV-2 viral challenges, animals were necropsied and the lower respiratory (lung) tissue specimens were collected, fixed, processed, and embedded in paraffin blocks and sectioned at a thickness of 5 μm. The sections were stained with hematoxylin and eosin (H & E) and examined by light microscopy. Multiple sections of lung and lymph node (axillary and inguinal) were evaluated histologically and immunohistochemically for the presence of SARS-CoV-2-related inflammation and SARS-CoV-2 virus antigen, respectively. A Rabbit polyclonal SARS-CoV-2 antibody (GeneTex) was used for immunohistochemical staining.

The inflammatory cellular constituents were largely similar for all groups where inflammation was observed (mixed polymorphonuclear and mononuclear cells) so, severity is based on % tissue affected and the presence or absence of other indicators of inflammation and tissue damage (fibrin/edema/luminal debris/hemorrhage/necrosis). In addition to lesion severity, lesion distribution and the location were recorded; lesions were either associated with/exhibited as alveolar interstitium (Alv) changes; intra-alveolar infiltrates (intraAlv); changes associated with Bronchi (Br) or Bronchioles (br); Perivascular spaces (PV) or exhibited variable degrees of Type II pneumocyte hyperplasia (Type II). Inflammation in the lung was scored using the following severity scale: normal=−(0); <10% (tissue affected)=+/−(1); >10−<25%=+(2); >26−<50%=++(3); >50%=+++(4). Three parts of the lung (Left caudal [Lc], Right Middle [Rmid], and Right caudal [Rc] lobes) were evaluated and scored by a board-certified veterinary pathologist, who was blind to the groups. The total inflammation score was calculated as the sum of the three parts. Sections were evaluated using an Olympus BX51 brightfield microscope and representative photomicrographs were captured using an Olympus DP73 camera.

Statistical analysis. Statistical analyses were performed using Prism version 8 (Graph Pad). Mann-Whitney, and Wilcoxon tests were used for group comparisons, and Spearman analyses were used for correlations, as shown in the figures. All statistical tests were 2 tailed. A P value of less than 0.05 was considered significant.

### Example 2

### Humoral Responses after Adjuvanted Systemic and Mucosal Subunit Vaccines

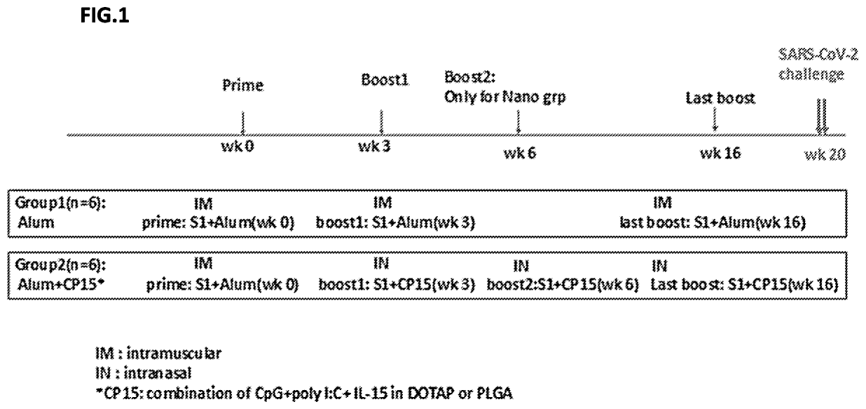

Two groups of 6 Indian rhesus macaques each were included to test the immunogenicity of the two vaccine platforms. The systemic vaccine was IM-primed and boosted with recombinant S1 protein in alum (group 1-alum group), while the mucosal vaccine was IM-primed with S1 in alum, and IN-boosted with S1-adjuvanted with a combination of IL-15 and TLR agonists (CpG and poly I:C) incorporated in PLGA or DOTAP nanoparticles (group 2-CP15 group). 100 μg of wild type S1 protein per dose was used in both vaccines. All the animals were primed at week 0 and boosted at week 3 (FIG. 1). An extra IN-boost was given to group 2 at week 6. The first two IN boosts were in PLGA nanoparticles. Sixteen weeks after the first vaccination, 25 days before SARS-CoV-2 viral challenges, both group 1 and 2 were boosted with S1 adjuvanted with either alum (IM) or CP15 in DOTAP nanoparticles (IN), respectively.

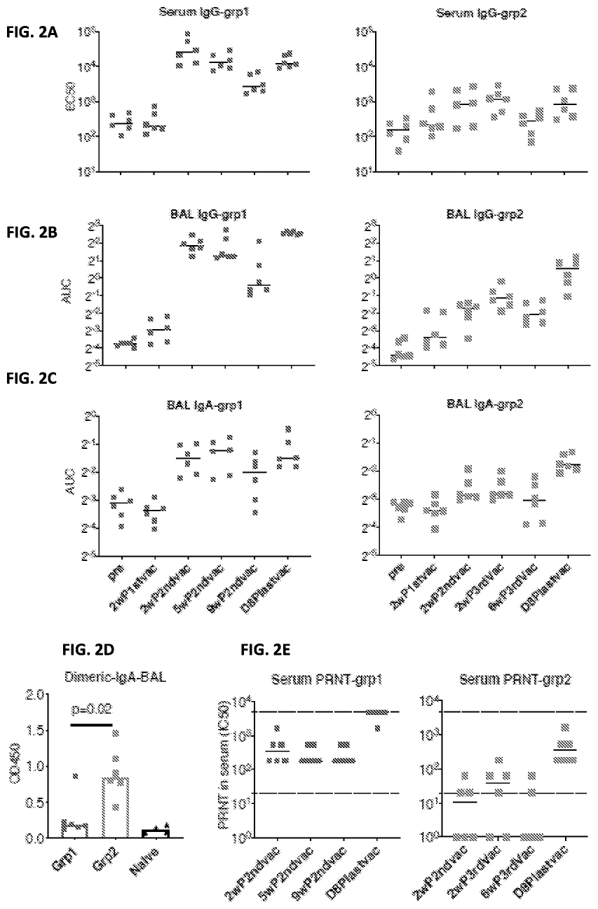

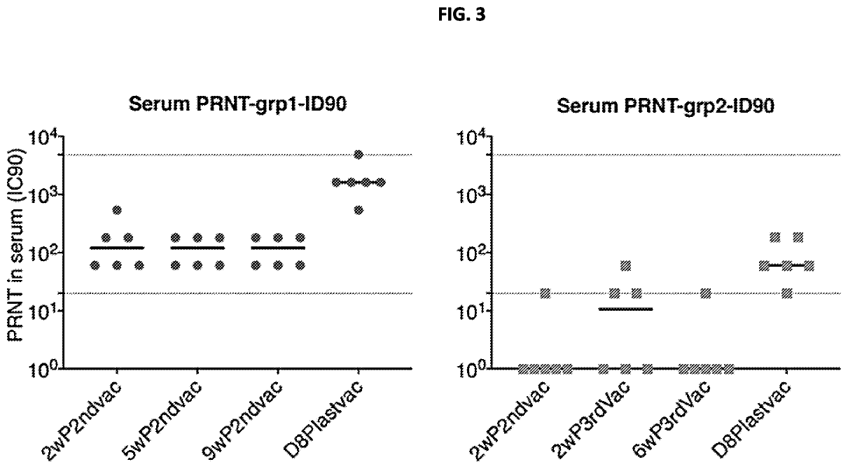

As antibodies have been proposed to be the major protective mechanisms for most vaccine strategies^{2,4,5}, the S1-specific antibody responses were evaluated in serum and bronchoalveolar lavage (BAL) fluid by ELISA (FIGS. 2A-2C). The first vaccination did not induce significant humoral responses over baseline in either platform. Two weeks after the second vaccination, robust S1-specific antibody responses, including serum IgG, BAL mucosal IgG and IgA, were elicited in group 1 animals, while much lower serum IgG and barely any BAL IgG and IgA responses were detected in group 2 animals (FIGS. 2A-2C). Group 1 reached a median serum EC50 of 25,209, while group 2 was significantly lower at 845 (FIG. 2A). The IgG and IgA titers in BAL followed similar patterns (FIGS. 2B-2C). No significant boosting anamnestic effects were observed even with an extra intranasal boost at week 6 for group 2. In group 1, declining antibody titers were observed in serum and BAL over the time, with about 10-fold decrease of serum IgG titer (to 2,596) at 9 weeks, compared to the peak at 2 weeks post second vaccination.

Sixteen weeks after the first vaccination, an IM-alum booster dose was given to group 1 and an IN-CP15 booster dose in DOTAP was given to group 2 animals, leading to a significant anamnestic increase of serum IgG titer to 11,977 in group 1 and 824 in group 2 (FIG. 2A). This last vaccination also resulted in the induction of mucosal IgG and IgA in BAL in group 2 (FIGS. 2B-2C). Nevertheless, after this boost, group 1 still had higher IgG responses in serum and BAL compared to those in group 2. Both groups had similar IgA responses in BAL. Dimeric IgA present at the mucosal surface has higher binding affinity to pathogens, and therefore is more potent than monomeric IgA, and thus may provide greater protection against mucosal pathogens^{23,24}. S1-specific dimeric IgA responses were assessed in BAL samples. Notably, group 2 had significantly higher dimeric IgA in BAL than group 1 (roughly 5-fold) post the last boost (FIG. 2D). All but one animal in group 1 had the same level of dimeric IgA as naïve controls. This indicated that the total S1-specific IgA responses were different in the two groups with group 2 having mainly dimeric IgA and group 1 having monomeric IgA. The higher dimeric IgA responses in the lung mucosa of macaques receiving the mucosal vaccine may provide better protection against viral challenges with SARS-CoV2 than the monomeric IgA responses.

All animals in group 1 had substantial neutralizing antibody (Nab) titers against live virus measured by plaque reduction neutralization test (PRNT) at 2 weeks post the second vaccination, while only 3/6 animals had detectable Nab titers in group 2. The geometric mean titer (GMT) of Nab IC50 was 374 in group 1, and 18 in group 2 (FIG. 2E). Interestingly, though the binding antibody titer in serum had a 10-fold decrease from 2-week to 9-week post second vaccination, the PRNT titers maintained similar levels (FIG. 2E). Day 8 after the last boost, even though the S1-binding antibody titer (11,977 and 824 for group 1 and 2 respectively) was still lower than or comparable to that of the 2-week post second vaccination level (25,209 and 845, respectively), the IC50 of PRNT in group 1 (GMT>4,047) was so high that 5 out of 6 animals exceeded the upper detection limit of 4,860. The GMT of PRNT in group 2 was also increased to 374. The ID90 of the PRNT data followed the same trend (FIG. 3). Thus, the two platforms of S1 subunit vaccines induced robust S1-specific antibody responses in blood and BAL, including potent neutralizing capacity in blood. Based on the prior challenge studies using macaque models, protective effects were usually observed in animals with PRNT titers higher than 100. The serum Nab titers of both groups were higher than or comparable to those induced by other platforms tested in macaque models.

Notably, the last vaccination played a pivotal role in increasing the Nab titers for both groups. Since PLGA nanoparticles were hard to suspend, and therefore hard to administer IN, DOTAP nanoparticles were used for the last boost, which might partially account for the elevated humoral responses following the last IN dose in group 2. Though the mechanisms are not known, it is possible that the interval of 2-3 months between the vaccination doses might give the antibody-producing B cells more time to interact with antigen-specific T helper cells and thus facilitate B cell maturation to high affinity/neutralizing antibody producing plasma cells. Hence, whether the vaccines could induce high quality antigen-specific T helper cell responses was determined

### Example 3

### Cellular Responses after Adjuvanted Systemic and Mucosal Subunit Vaccines

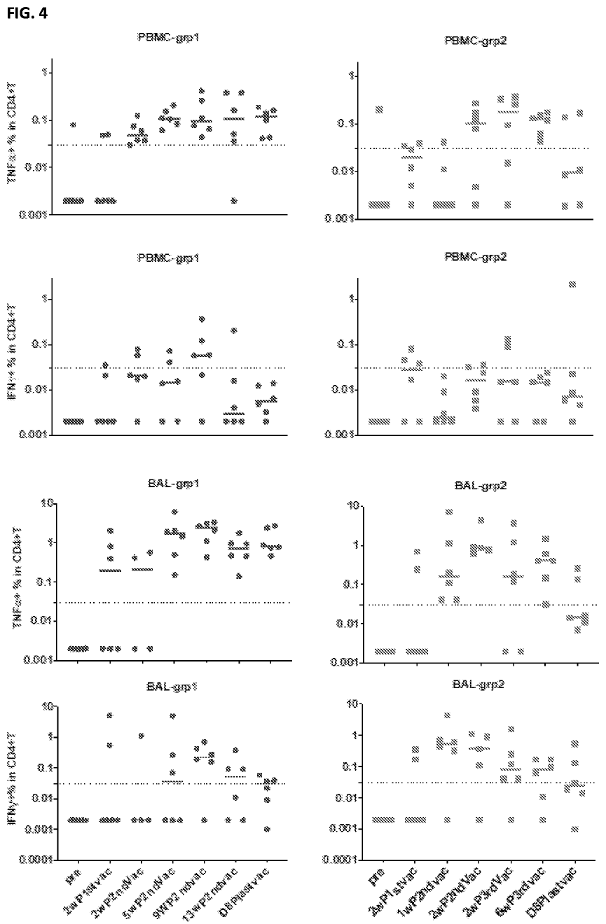

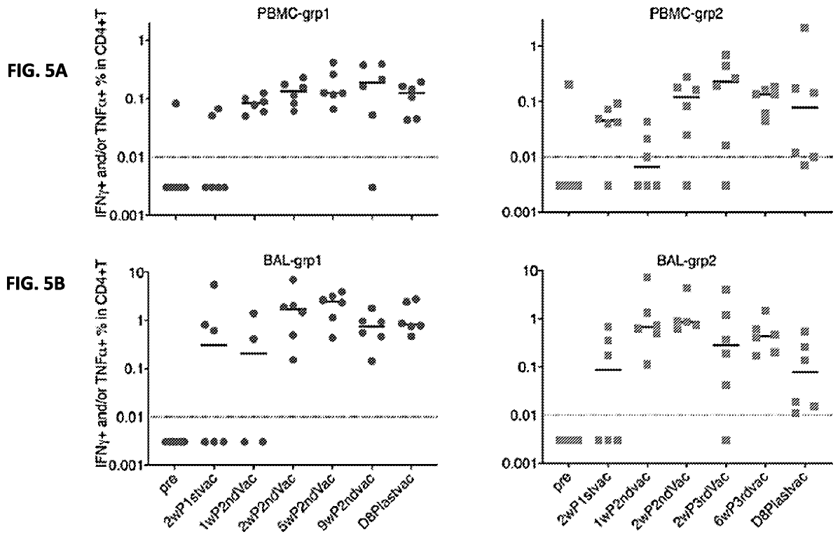

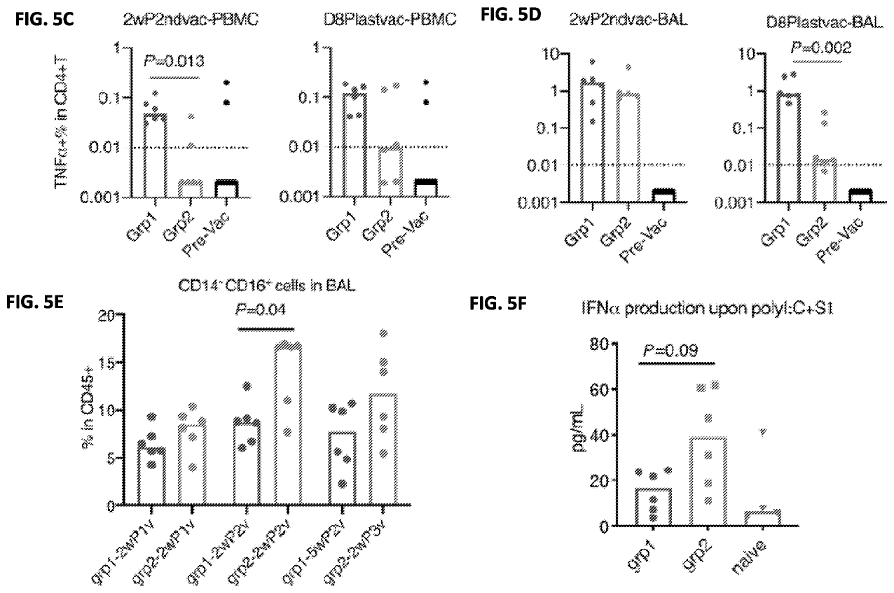

Vaccine-induced S1-specific T cell responses were evaluated throughout the whole course of vaccination. Even though the role of SARS-CoV-2-specific T cell responses in COVID-19 is still unclear, viral-specific CD4+ T cells can provide help for B cell activation, maturation and antibody induction. Type 1 helper T cell responses (Th1) that secrete tumor necrosis factor (TNF)-α, and/or interferon (IFN)-γ are critical for this process. Different subsets of S1-specific T helper and CD8⁺ T cell responses were measured in the PBMC and BAL samples of the vaccinated animals. Th1 responses were not induced until after the second vaccination. Both in PBMC and BAL, the dominant Th1 responses were TNF-α-secreting cells (FIG. 4). In group 1, the Th1 responses were persistent throughout the whole study in PBMC and BAL samples, while in group 2, the responses were durable in BAL, but not in PBMC (FIGS. 5A-5B). Group 1 animals had higher Th1 responses in the PBMC than those in group 2 at both early and later time-points during the vaccination sequence (FIG. 5C). Similar Th1 responses in BAL were seen in both groups at early time-points but dropped significantly in group 2 at later time-points despite the mucosal immunizations that group 2 animals received (FIG. 5D). The decrease might be attributed to the migration of the antigen-specific cells to the upper respiratory tracts after IN vaccination.

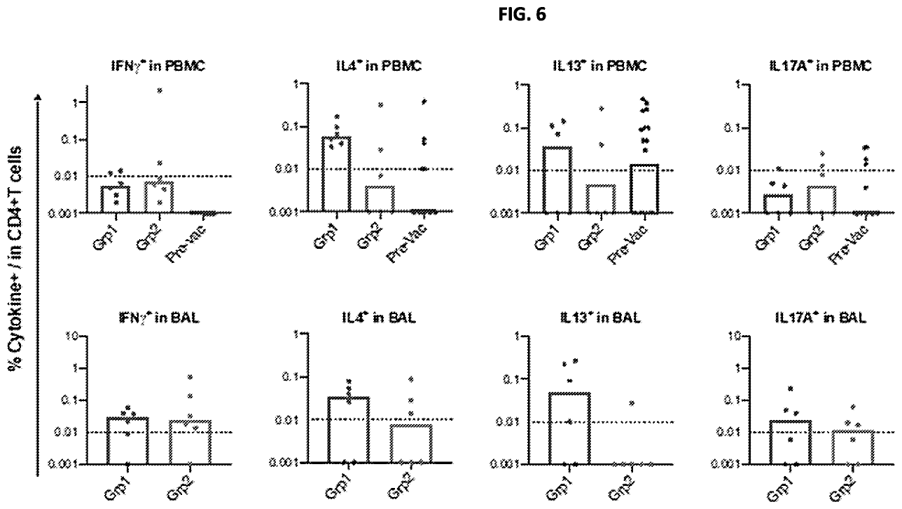

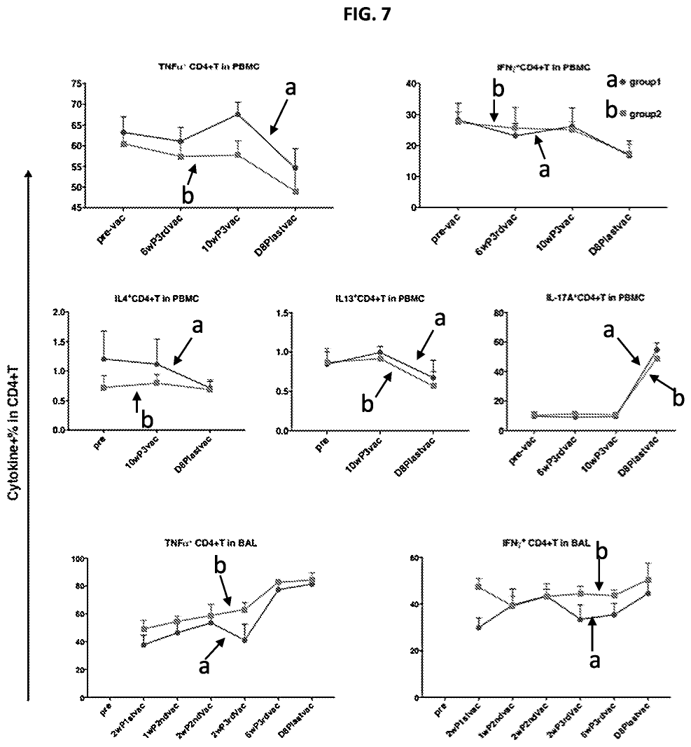

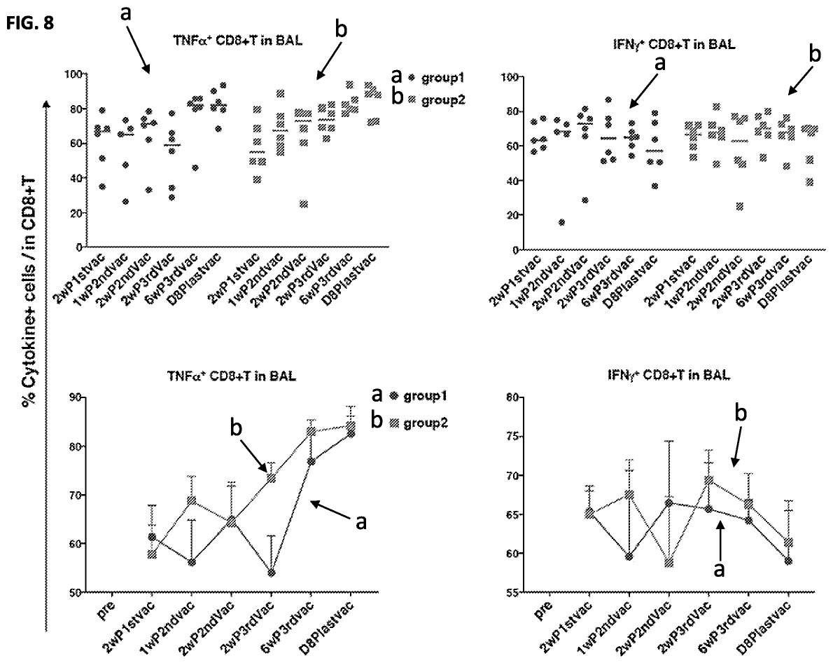

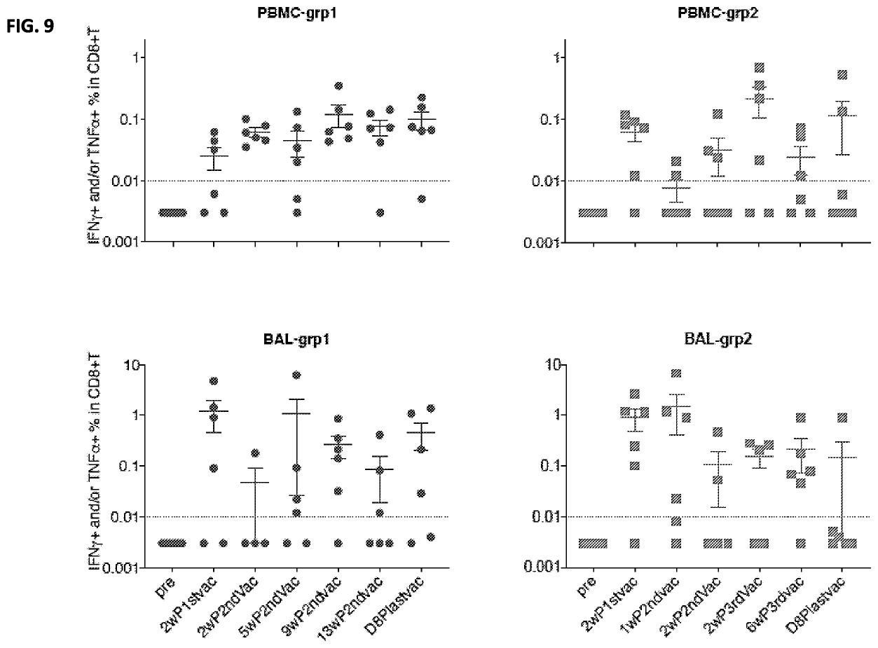

In other viral respiratory infections, including SARS-CoV, middle east respiratory syndrome coronavirus, the presence of Th1 responses is more favorable to control disease, while the induction of Th2 and Th17 responses has been linked to immunopathogenic lung diseases in animals or clinical trials. When evaluating S1-specific Th2 (IL-4-, IL-13-secreting cells), and Th17 (IL-17A-secreting cells) responses, no significant differences between the 2 vaccinated groups after the vaccination or in the prevaccination levels (FIG. 6). However, since the frequencies of antigen-specific T cell responses were low, the kinetics of total Th1, Th2, and Th17 subsets were analyzed after stimulating the samples with Phorbol 12-myristic 13-acetate (PMA) and ionomycin. In these more robust assays, a slight down-trend of Th1, an up-trend of Th17, and no change for Th2 in PBMC was observed (FIG. 7). This was in sharp contrast to the scenario in BAL, where Th1 response increased over time, and especially the frequency of TNF-α-secreting cells was almost doubled compared to pre-vaccination levels (from 40% to 80%) (FIG. 7). Total TNF-α-secreting CD8⁺ T cells (Tc1) also increased markedly from 60% to 85% after stimulation with PMA and ionomycin (FIG. 8). This increase of Th1 and Tc1 responses in BAL for both vaccine platforms indicated that a re-distribution of the T helper and CD8⁺ T subsets might occur during the vaccination. The high frequency of Th1 and Tc1 subsets in the BAL might be beneficial to the host. The S1-specific CD8+ T cell responses were also induced in some of the vaccinated animals from both groups, but with less magnitude and persistence (FIG. 9).

A similar platform was used with TLR agonists plus IL-15 as adjuvants to develop an HIV vaccine, where trained innate immunity was induced and was involved in mediating protection against viral transmission. Trained immunity is characterized by enhanced innate responses after encounter with the pathogens the second time, and this is usually achieved through epigenetic modification of genes in myeloid or natural killer cells. The frequency of changes of CD14+ and/or CD16+ populations was measured in BAL. The CD14⁻CD16⁺ population showed a trending increase in group 2 compared to those of group 1 and also increased compared to samples before receiving the CP15 adjuvants (P=0.002; FIG. 5E). However, more boosting (third vaccination) did not further increase the frequency of these cells (FIG. 5E).

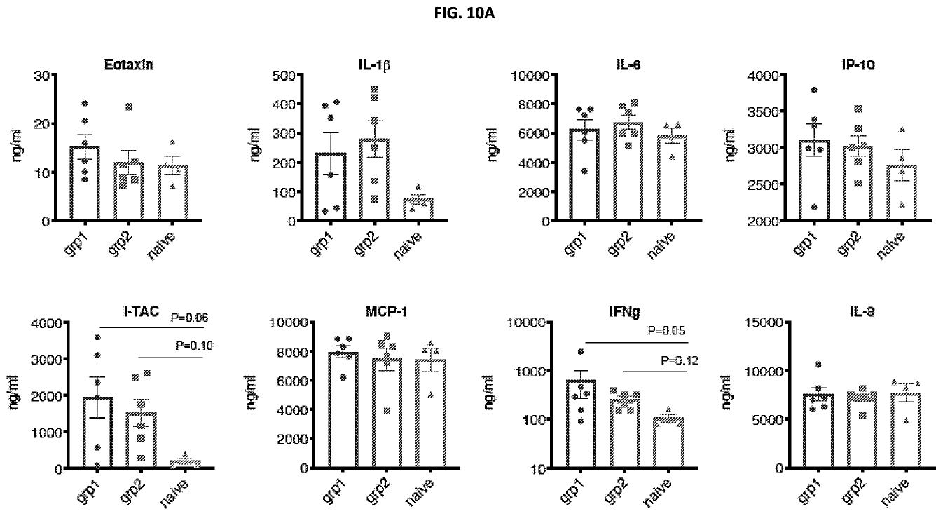

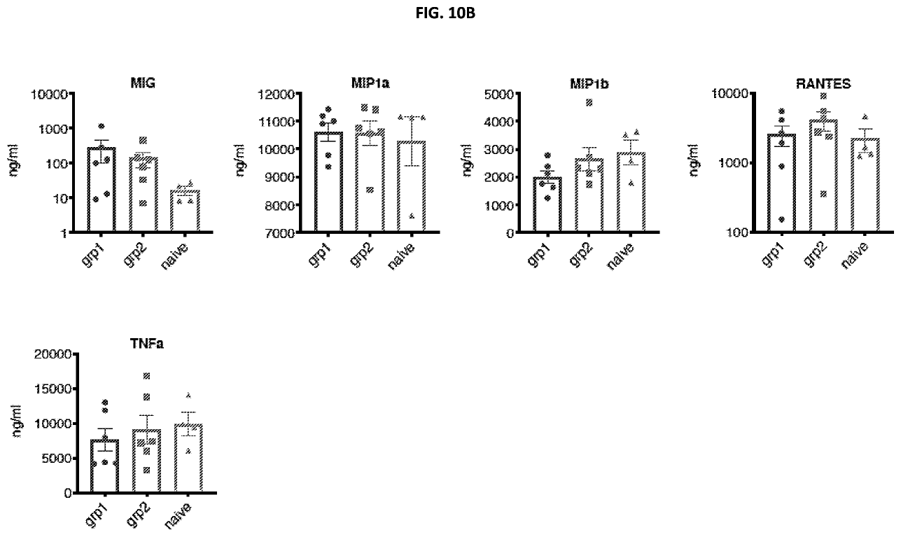

IFN-α expression levels were measured in BAL samples after exposure to the viral mimic: Poly I:C plus S1 protein. Because the small BAL samples collected at later time points were used up for antigen-specific T cell responses, 1 week after vaccination BAL samples were used to measure IFN-α expression. Upon stimulation with poly I:C and S1 protein ex-vivo, the BAL samples from group 2 produced higher levels of IFN-α in the supernatant than those of group 1 or the naïve group (FIG. 5F), while other cytokines and chemokines did not differ significantly between the groups (FIGS. 10A-10B). These data indicate that trained innate immunity, represented by the CD14⁻/CD16⁺ subpopulation and the production of IFN-α upon stimulation, was induced by S1 with CP15 adjuvant (CpG, poly I:C plus IL-15).

### Example 4

### Viral Load in Nasal Swab and BAL Samples after IN and Intratracheal Routes of SARS-CoV-2 Viral Inoculations

To demonstrate vaccine efficacy, about 4 weeks after the last vaccination, the 12 vaccinated and 6 naïve macaques were challenged with 1.5×10⁴ PFU SARS-CoV-2 virus (USA-WA1/2020 strain), which was equivalent to approximately 1.25×10⁵ tissue culture infectious dose 50 (TCID₅₀). The challenge virus was obtained from BEI Resources and has a reported infectious titer in Vero E6 cells of 3×10⁶ PFU/mL. The dose was chosen to be approximately the same (1.1×10⁴ PFU) as the dose established by the earlier studies carried out at the same facility (BIOQUAL Inc.). The animals were challenged via both IN and intratracheal routes in order to deliver the virus to both upper and lower airways simultaneously. Genomic RNA (gRNA) and subgenomic RNA (sgRNA) PCRs were performed to quantify the input and replicating virus respectively. SgRNA in particular is an indication of replicating virus.

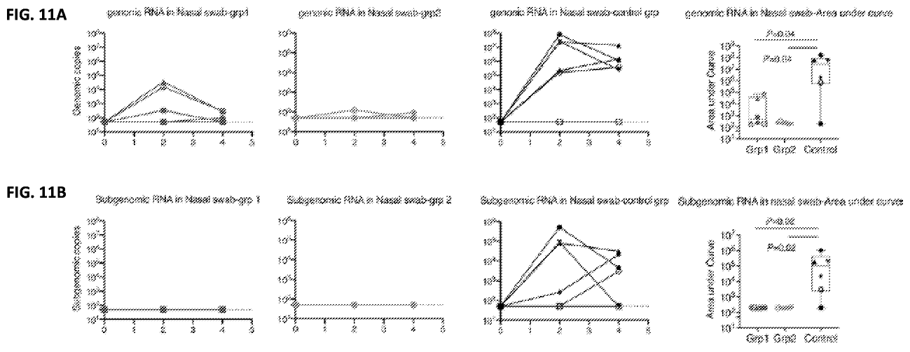

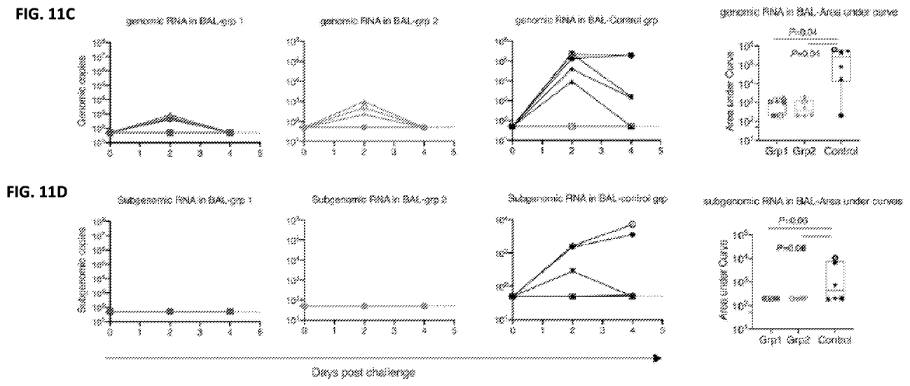

After viral challenge, 5 out of 6 SARS-CoV-2-naive control animals demonstrated clear signs of viral replication, shown by sgRNA viral load (VL). Among the 5 infected animals, 3 animals had viral replication in both nasal swabs and BAL fluid, and 2 animals had sgRNA in nasal swabs but not in BAL fluid (FIGS. 11A-11D). Similar to other studies of SARS-CoV-2 vaccines in macaque models, the input VLs were much higher than the replicating VLs. At day 2, a VL of log 7 in nasal swabs and a VL of log 5 in BAL fluid were detected. One animal, DFKL, in the naive group, did not shown any signs of infection. Even the input virus, as shown in gRNA VL, was negative in all samples tested. DFKL previously had been exposed to 8 repeated challenges of SIVmac251, but never showed any VLs for SIV, suggesting that this animal might have unique innate immunity, which allowed it to quickly clear the input virus. Indeed, this animal had unusually high levels of IFN-α, SCF, I-TAC, IL-1R-α, and PDGF-BB in serum. The high level of IFN (undetectable in naive uninfected samples) might explain the resistance of DFKL to SIVmac251 and SARS-CoV-2 viral challenges.

In the vaccinated groups, through the whole course of infection, sgRNA in the nasal swabs and lung fluid was not of any animals (FIGS. 11B and 11D). These data demonstrate that both vaccine platforms mediated 100% protection against replicating virus in both tissues, which has been rarely seen with previous COVID-19 vaccines in macaques. Even the input virus gRNA was rapidly cleared in the nasal swabs of 3 of 6 in group 1 and 5 of 6 in group 2 animals already at day 2 after infection. In the BAL fluid, the input virus was also cleared in 2 group 1 and 3 group 2 animals at day 2, and all were cleared by day 4 (FIGS. 11A and 11C).

### Example 5

### Immune Correlations of Humoral and Cellular Responses after Vaccination and Viral Challenges

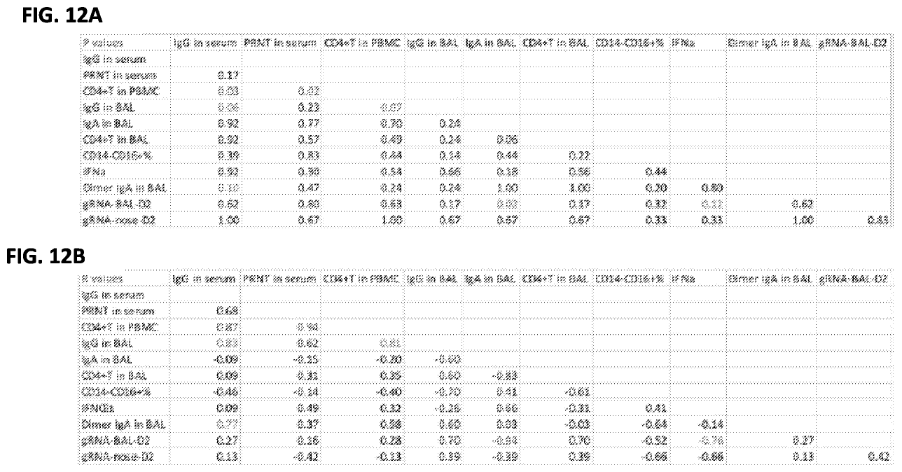

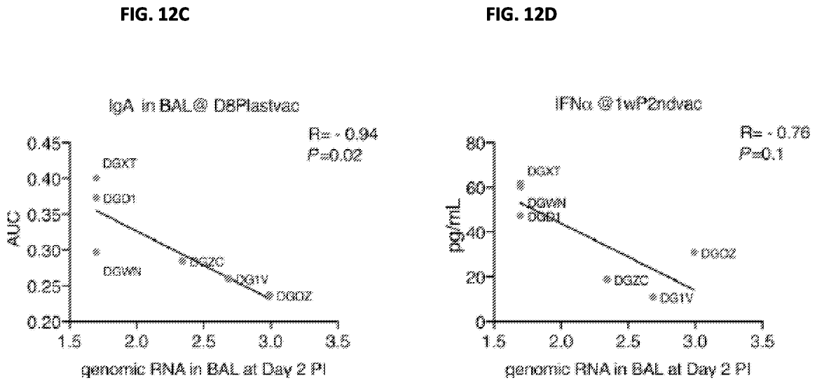

Since full immunity against sgRNA had been achieved for both vaccines, the immune correlates of protection at the sgRNA level could not be identified. However, the immune correlates with peak gRNA data after the mucosal vaccine were analyzed, which is a surrogate marker of efficiency of clearance of input virus. Since group 1 and 2 animals had different immune responses and might have different protection mechanisms, it was more logical to analyze them separately in order to have the capability to compare between the 2 groups. Since most of the immune responses in group 1 were very similar to each other, there was not enough spread to find significant correlations within that group. Several significant correlations or trends of significance in group 2 were observed (FIGS. 12A and 12B). Notably, both serum S1-specific IgG and PRNT responses positively correlated with antigen-specific CD4+ T cell responses in PBMCs (R=0.94 and 0.87; P=0.02 and 0.03, respectively), indicating the importance of antigen-specific Th1 responses to induce humoral responses. gRNA in BAL inversely correlated (or showed a trend) with S1-specific IgA titers and IFN-α production in BAL samples (FIGS. 12C and 12D, R=−0.94 and −0.76; P=0.02 and 0.12, respectively), indicating that local respiratory mucosal immunity might participate in clearing of the input virus more efficiently. No correlation was observed between dimeric IgA and gRNA clearance after viral challenge. It is possible that whereas dimeric IgA, as an ideal mucosal defender, can efficiently neutralize virus by immune exclusion to prevent the virus from contacting epithelial cells, or trapping the invaders on the luminal surface, dimeric IgA is a poor opsonin and a weaker activator of complement system and thus is not capable of clearing the virus-Ab complexes as quickly as IgA does. However, the higher dimeric IgA titers in group 2 (FIG. 2D) may contribute to inhibiting viral replication by preventing the virus from infecting the target cells. Thus, both mechanisms may play a role.

### Example 6

### Histopathology after Viral Infection

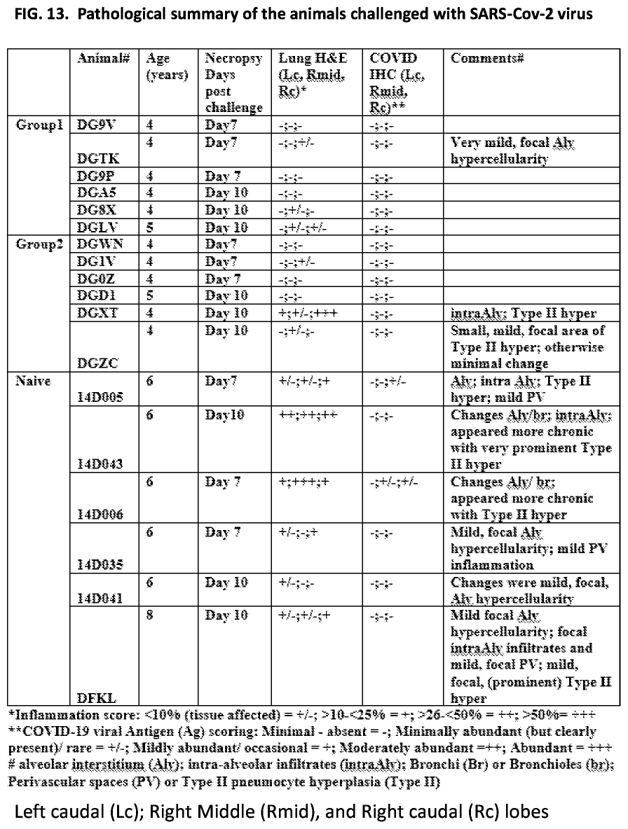

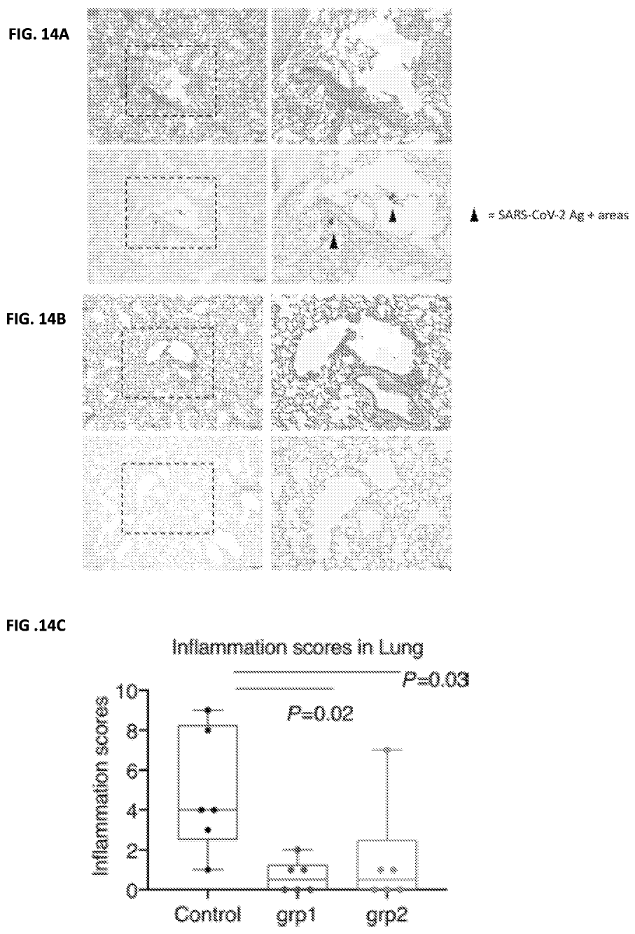

Throughout the study, no clinical abnormalities were observed in the control and study group animals. Necropsies were performed on either day 7 or 10 (FIG. 13). One-half of the animals in each group were euthanized on day 7 and the other half on day 10. The distribution is evenly divided, and therefore, the histopathology results/lung inflammation scores are comparable. The timing was also dependent on the need to first collect BAL fluid on days 2 and 4 after challenge. Sections of lung and lymph node (axillary and inguinal) from animals necropsied on day 7 were evaluated histologically and immunohistochemically for the presence of SARS-CoV-2-associated inflammation and SARS-CoV-2 virus antigen, respectively. Most lung sections were negative for virus antigen immunoreactivity, but in some cases, rare positive foci of virus antigen were observed in samples from 2 control animals (FIGS. 14A and 14B). The severity of inflammation, when present, ranged from mild to moderate severity. The inflammatory changes observed were characterized by a mixed polymorphonuclear and mononuclear (predominantly macrophage) cellular infiltrate present within alveolar capillaries and, less frequently, present within the alveolar spaces. Inflammatory lesions were most associated with regions surrounding small bronchioles and small-caliber blood vessels. Perivascular infiltrates were largely composed of small lymphocytes and fewer histiocytes. Significant inflammation was largely absent in the sections of lung examined for this cohort. Each animal was given an inflammation score based on the evaluation of lung infiltration (FIG. 13). In accordance with the VL data, the scores from the SARS-CoV-2 naive control group were significantly higher than those from the vaccinated groups (FIG. 14C). There was no evidence of significant inflammation or virus antigen observed in the sections of lymph node examined. The 2 naive animals that showed positive virus antigen staining in the lung had the highest gRNA VL and highest inflammation scores, consistent with the fact that the inflammation was induced by viral infection. However, we also observed prominent lung inflammation from 1 vaccinated animal from group 2, which did not show any gRNA or sgRNA in either nasal swabs or BAL at any time points tested, indicating the inflammation was sometimes induced by factors other than viral infection. Interestingly, the only animal that did not become infected in the naive group also demonstrated a certain level of inflammation in the lung (FIG. 13).

### Example 7

### Materials and Methods

This example describes the materials and method used to generate the data described in Example 8.

Animals. 10 Indian-origin adult male rhesus macaques (Macaca mulatta), 3-8 years old, were enrolled in the study. The animals tested seronegative for cercopithecine herpesvirus 1, SIV, simian type-D retrovirus, simian T lymphotropic virus type 1, and SARS-CoV-2 prior to study assignment.

Vaccine design and inoculation. Five previously primed male macaques were included in the vaccine group. Since during the COVID-19 pandemic, we were not able to obtain matched males, five female macaques were included in the SARS-CoV-2-naive control group. If anything, female macaques would be expected to make stronger immune responses than males, so the gender difference would not account for any greater immune response in the vaccinated male animals. The five naïve control animals had been exposed to HIV envelope protein/glycopeptide vaccination more than one year before, but had not been infected or challenged. The five macaques in the vaccine group were primed at Week 0 (administrated IM) and boosted at Week 3 (administered IN) and Week 6 (administered IN) with SARS-CoV-2 S1 protein (WA strain) with alum or CP15 adjuvant in PLGA nanoparticles. The CP15 adjuvant was composed of 200 μg per dose of D-type CpG oligodeoxynucleotide, 1 mg per dose of Poly I:C (InvivoGen), and 200 μg per dose of recombinant human IL-15 (Sino Biological). One year later, a boost was given to the remaining five animals with S1 protein from the beta variant adjuvanted with CP15. 100 μg of recombinant SARS-CoV-2 (2019-nCoV) spike S1 protein (Cat: 40591-V08H and 40591-V08H10, Sino Biological, endotoxin level: <0.001 U/μg) was used per dose. S1 protein and CP15 were formulated in nanoparticles in PLGA (Alchem Laboratories) for the first 2 doses and the last (one-year) boost was in DOTAP (100 μl per dose; Roche). For immunization, the CP15 adjuvanted vaccine was given either intramuscularly in 1 ml of volume, or intranasally in a volume of 50 μl per nostril, while the animals were anesthetized. After vaccination, blood, nasal swab and BAL fluid samples were collected at the times noted and analyzed.

Nasal swab and BAL sample collection. Nasal secretions were collected and stored at −80° C. after either using cotton-tipped swabs and then in 1 ml of PBS buffer containing 0.1% BSA, 0.01% thimerosal, and 750 Kallikrein inhibitor units of aprotinin for pre-challenge stage, or using Copan flocked swabs and in virus transport medium for post-challenge stage. BAL samples were collected. Briefly, while the animals were under anesthesia, up to 10 mL/kg of sterile saline were instilled into and sucked out of the lungs. Large pieces were removed by passing through a 100 μm cell strainer (pre-challenge). The BAL fluid as collected after centrifugation and stored at −20° C. for analysis. The BAL cells were washed with RIO medium (RPMI-1640 with 10% fetal bovine serum) before subsequent treatment or cryopreservation.

ELISA assay to detect S1-specific antibody responses. The BAL samples were concentrated using Amicon Ultra centrifugal filter units (10 kDa cutoff, Millipore Sigma), and the total IgG and IgA were determined using the Rhesus Monkey IgG-UNLB (Southern Biotech), and the Monkey IgA ELISA development kit (HRP) (MabTech) respectively, following the manufacturer's protocol. Nasal swab samples were put into 1 ml of 1×PBS buffer containing 0.1% BSA, 0.01% thimerosal, and 750 Kallikrein inhibitor units of aprotinin (Sigma) and stored at −80° C. Nasal swabs were thawed, and the recovered solution was passed through 5 μm PVDF microcentrifugal filter unit (Millipore, Billerica, MA). The buffer flow-through was collected and stored at −20° C. until analysis. ELISA assays were run.

Plaque reduction neutralization test (PRNT). The PRNT was performed in duplicate as described in Example 1. Vero E6 cells (ATCC, cat. no. CRL-1586), and 30 pfu challenge titers of SARS-CoV-2 virus USA-WA1/2020 strain or Vero TMPRSS2 cells (obtained from Dr. Adrian Creanga and Barney Graham, VRC, NIAID, Bethesda, MD) and same titer of the beta variant (B.1.351, SRA strain) was used to test the PRNT titers against the WA or beta variant of SARS-CoV-2. Serum samples of 3-fold serial dilution starting from 1:20, and up to final dilution of 1:4860 were incubated with 30 pfu of SARS-CoV-2 virus for 1 hr. at 37° C. The serial dilutions of virus-serum mixtures were then added onto Vero E6 cell monolayers in cell culture medium with 1% agarose for 1 hr. at 37° C. with 5% CO2. The plates were fixed and stained after three days of culture. ID50 and ID90 were calculated as the highest serum dilution resulting in 50 and 90% reduction of plaques, respectively.

Intracellular cytokine staining assay. SARS-CoV-2-specific T cells were measured from BAL and PBMC samples by flow cytometric intracellular cytokine analysis, and the detail protocol was in the supplemental material. The antigen-specific T cell responses were reported as the frequencies of cytokine-positive cells in the samples stimulated with S1 protein minus those in the medium-only control from the same animal at each time-point. If the medium control had a higher frequency of cytokine-positive cells than that of the S1 protein-stimulated sample in the matched animal, an arbitrary number of “0.001” was assigned to each cytokine as a negative on the log scale. SARS-CoV-2 beta variant viral challenge. Four weeks after the one-year boost, 5 vaccinated and 5 control animals were challenged with 1×10\hat{ } pfu SARS-CoV-2 virus beta variant (seed stock obtained from BEI Resources; NR-54974, B.1.351, SRA strain). The challenge stock was grown in Calu-3 cells and was deep sequenced, which confirmed the expected sequence identity with no mutations in the Spike protein greater than >2.5% frequency and no mutations elsewhere in the virus at >13% frequency. The same beta variant stock was used in the earlier macaque challenge study at the same facility. To make sure that the virus was delivered to both upper and lower airway simultaneously, the diluted virus was given intranasally and intratracheally, each route with 1 ml (0.5 ml for each nostril). Nasal swab and BAL fluid samples were collected after challenge to measure the viral load.

TCID50 assays to measure viral loads. Vero TMPRSS2 cells (obtained from the Vaccine Research Center-NIAID) were plated at 25,000 cells/well in DMEM+10% FBS+Gentamicin and the cultures were incubated at 37° C., 5.0% CO2. Cells should be 80-100% confluent the following day. Medium was aspirated and replaced with 180 μL of DMEM+2% FBS+gentamicin. Twenty (20) μL of sample was added to top row in quadruplicate and mixed using a P200 pipettor 5 times. Using the pipettor, 20 μL was transferred to the next row, and repeated down the plate (columns A-H) representing 10-fold dilutions. The tips were disposed for each row and repeated until the last row. Positive (virus stock of known infectious titer in the assay) and negative (medium only) control wells were included in each assay set-up. The plates were incubated at 37° C., 5.0% CO2 for 4 days. The cell monolayers were visually inspected for CPE. Non-infected wells will have a clear confluent cell layer while infected cells will have cell rounding. The presence of CPE was marked on the lab form as a + and absence of CPE as −. The TCID₅₀ value was calculated using the Read-Muench formula. For optimal assay performance, the TCID50 value of the positive control should test within 2-fold of the expected value.

Histopathology and immunohistochemistry of lung sections. Seven days after SARS-CoV-2 viral challenge all the animals were necropsied and the lung tissue specimens were collected, fixed, processed, and embedded in paraffin blocks and sectioned at a thickness of 5 μm. Briefly, hematoxylin and eosin (H & E) sections were examined under light microscopy and scored by a board-certified veterinary pathologist, who was blind to the groups. A rabbit polyclonal SARS-CoV-2 antibody (GeneTex) was used immunohistochemically to stain for the presence of SARS-CoV-2 virus antigen. An Olympus BX51 brightfield microscope was used, and representative photomicrographs were captured using an Olympus DP73 camera.

Statistical analysis. Prism version 8 (Graph Pad) was used for statistical analyses. Area under curve (AUC) values were calculated for viral load, and Mann-Whitney and paired t tests were used for group comparisons as shown in the figures. A P value less than 0.05 was considered significant, and all statistical tests were 2-tailed.

ELISA assay to detect S1-specific antibody responses. The S1-specific binding assays were coated with 100 ng/well of the SARS-CoV-2 spike S1-His Recombinant Protein (Sino Biological) using high-binding 96-well plates (Santa Cruz Biotechnology). After incubation at 4° C. overnight, and 1 hr. blocking with 300 μL of 2% sodium casein in 1×PBS, the concentrated BAL samples (with a series of 2-fold dilutions starting from an IgA or IgG concentration of 2 μg/mL) or nasal swab samples, or serially diluted serum samples (4-fold starting from a 1:150 dilution) were applied in duplicate. After incubation at room temperature for 1 hr., the plates were washed four times. Subsequent steps of incubation with HRP-labeled secondary antibody and TMB substrate were followed as described before. For IgG and IgA binding assay, Goat Anti-Monkey IgG (alpha-chain specific)-HRP conjugate (1:5,000 dilutions, Alpha Diagnostic) and were used, respectively, as a secondary antibody. Area under the curve, endpoint titer, and half-maximal binding titers were calculated by GraphPad Prism 8 software with sigmoidal nonlinear regression. Dimeric IgA in BAL and nasal swabs was measured using DuoSet ELISA Ancillary Reagent Kit 2 (R&D Systems). 100 ng/well of the SARS-CoV-2 spike S1 protein was coated and blocked. Original BAL samples or nasal swab flow-through from vaccinated and naïve animals were added in duplicate to the plates, followed by adding mouse anti-rhesus J chain [CA1L_33e1_A1a3] antibody (1:1000 dilutions, NIH nonhuman primate reagent resource), and Goat anti-mouse IgG-HRP conjugate (1:10,000 dilutions, R&D Systems). Each step was followed by 1 hr. incubation at room temperature and five washes.

Intracellular cytokine staining assay. SARS-CoV-2-specific T cells were measured from BAL and PBMC samples by flow cytometric intracellular cytokine analysis. Briefly, 2 μg/ml of SARS-CoV-2 S1 protein (Sino Biological) for PBMC, and 5 μg/ml for BAL samples was incubated with cell samples at 37° C. 5% CO₂ overnight in the presence of 0.15 μg/ml of brefeldin A. Negative and positive controls were stimulated with medium-only (no S1 protein) or with cell activation cocktail with PMA (20.25 pM) and ionomycin (335 pM) and 0.15 μg/ml of brefeldin A (Biolegend). Cells were stained with viability dye (Invitrogen) and the following antibody mixtures: PE-Cy7-CD3, BV605-CD4, APC-Cy7-CD8, Alexa Fluor® 700—CD45 were from BD Biosciences, FITC-CD28, Pe-Cy5-CD95, BV711—TNFα, IFNγ-PE or—PerCP, Alexa Fluor® 647-IL4, BV785-IL2, BV421-IL-17A, BV785-CD14, BV421-CD16 were from Biolegend; PE-IL13 was from Miltenyi Biotech. Data acquisition and analyses were performed using an LSRII flow cytometer with 4 lasers (BD Bioscience) and FlowJo software (Becton Dickinson)

### Example 8

### Intranasally Administrated SARS-CoV-2 Beta Variant Subunit Booster Vaccine Prevents Beta Variant Replication in Rhesus Macaques

Emergence of novel SARS-CoV-2 variants of concern (VOC) threatens the efforts to curb the COVID-19 pandemic. Some variants demonstrated significantly reduced neutralization sensitivity to sera from convalescent and vaccinated individuals. A recent study assessed the cross-reactive neutralizing responses to different variants including B.1.1.7 (Alpha), B.1.351 71 (Beta), P.1 (Gamma), B.1.429 (Epsilon), B.1.526 (Iota), and B.1.617.2 (Delta) in mRNA-1273 vaccinated individuals, and found that the beta variant had the lowest antibody recognition. To date, the beta variant seems to be one of the most resistant variants to convalescent and vaccinated sera (surpassed only by the more recently identified omicron variant). Multiple mutations were found in this variant with K417N, E484K, N501Y as key substitutions. It had 5-fold enhanced affinity to ACE2 compared to the original virus, and from several—to up to 10-fold reduction in neutralization ability in convalescent and vaccinated individuals. Two studies have shown that the beta variant can partially or completely escape three classes of therapeutically relevant antibodies and convalescent sera.

Meanwhile, waning immunity after vaccination has led to a gradual decline of vaccine efficacy against SARS-CoV-2 infections. Recently, more SARS-CoV-2 breakthrough infections in vaccinated individuals, and resurgence of SARS-CoV-2 cases in some countries have been observed. Based on the previous experience with other coronaviruses and the current situation, an extra booster with the original Pfizer-BioNTech mRNA vaccine after 6-months of the first vaccination has been authorized in some countries among individuals with older age, high risk for severe COVID-19, or high risk for SARS-CoV-2 infections due to occupational or institutional exposure. For the general population, it is anticipated that a booster, ideally targeting circulating viral variants, will be needed, when the immunity induced by the original vaccine cannot provide adequate protection against the circulating viral variants. Since a large number of individuals have been vaccinated with the vaccines comprised of antigens from the SARS-CoV-2 original Wuhan strain, data on immunogenicity and protective efficacy of a variant booster to vaccines, who have previously received the original vaccines, would be urgently needed. Recent studies have shown that intranasal administration of different platforms of SARS-CoV-2 vaccines induce protective immunity in preclinical animal models.

Herein provided is a study to test the immunogenicity and efficacy of an adjuvanted SARS-CoV-2 beta variant subunit booster in rhesus macaques that were vaccinated with the same vaccine platform except that the spike protein S1 was from the original Wuhan strain. One year after the first vaccination, almost no detectable immunity was present in these macaques. However, an intranasal booster with the adjuvanted beta variant S1 subunit vaccine induced vigorous humoral and cellular immunity against both original and beta variant antigens. Secretory IgA responses against S1 from both the original Wuhan strain and the beta variant were detected in the nasal cavity, consistent with the almost full protection we observed against the beta variant in the nasal cavity after viral challenge. The data herein show that the one-year intranasal booster with beta variant S1 protein reinvigorated SARS-CoV-2-specific immune responses and led to significant protection against beta variant challenge.

Robust Systemic and Mucosal Humoral Responses Against S1 from the Original Wuhan Strain and Beta Variant were Elicited after Intranasal Variant Booster

In this example, we took advantage of five Indian rhesus macaques that had been vaccinated one year earlier with S1 protein from the original Wuhan strain (Table 1).

TABLE 1
Basic information of the animals tested

| Group | Animal ID | Sex | Date birth | Weight |
|---|---|---|---|---|
| Vaccine | DGVC | Male | May 1, 2016 | 5.7 |
| Vaccine | DGNi | Male | Mar. 18, 2016 | 6.5 |
| Vaccine | 1E | Male | Apr. 7, 2016 | 6.8 |
| Vaccine | DGZT | Male | Apr. 4, 2016 | 5.5 |
| Vaccine | DGLM | Male | Apr. 14, 2015 | 5.1 |
| Control | 14D044 | Female | Jun. 19, 2014 | 4.07 |
| Control | 14D064 | Female | Jun. 19, 2014 | 4.8 |
| Control | 14D080 | Female | Aug. 8, 2014 | 4.28 |
| Control | 14D065 | Female | Jun. 29, 2014 | 4.68 |
| Control | 14D068 | Female | Jul. 15, 2014 | 4.8 |

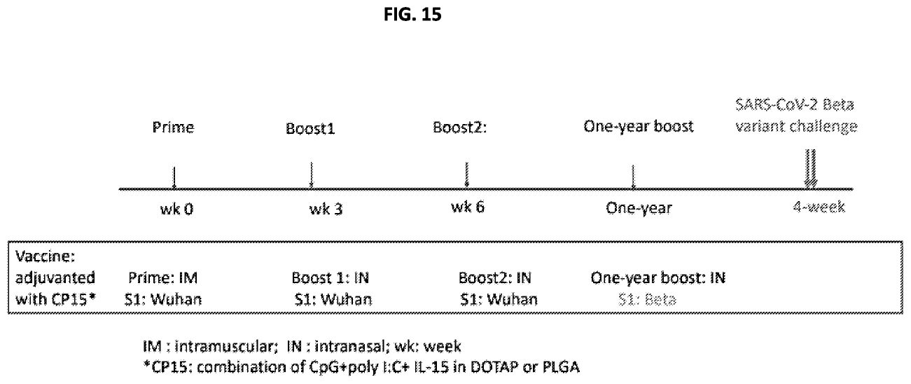

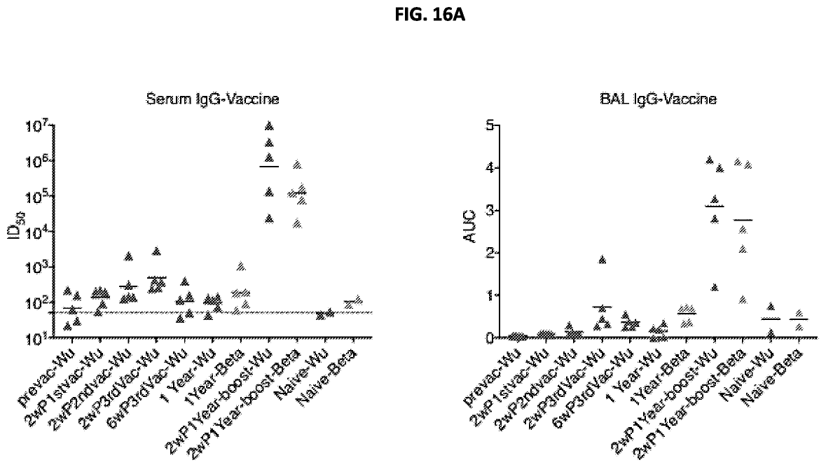

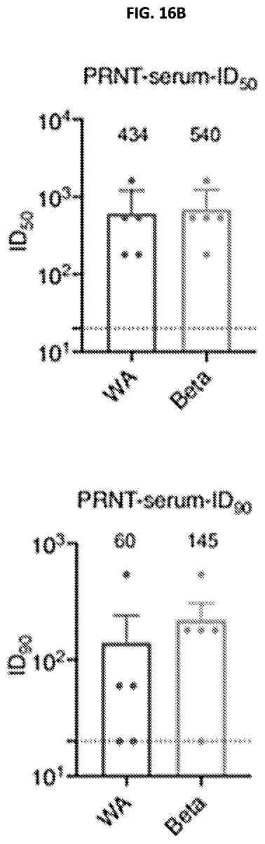

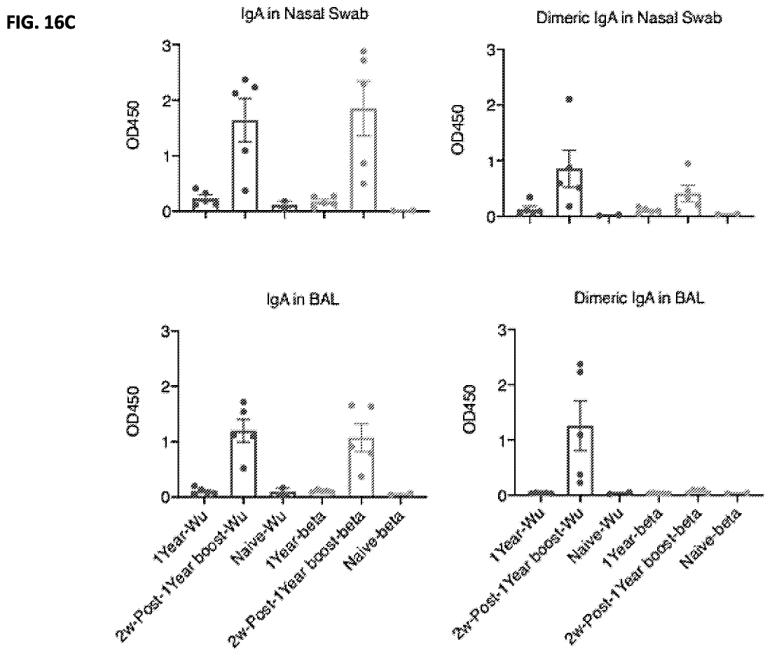

The vaccine included 100 μg of S1 and CP15 adjuvant, which was composed of IL-15 and TLR agonists (CpG and Poly I:C) incorporated in PLGA nanoparticles as used in Examples 1-6. The macaques were first primed with the vaccine intramuscularly (IM) at week 0, and then boosted with the same vaccine intranasally (IN) at week 3 and week 6 (FIG. 15). 100 μg of S1 per dose was used based on previous HIV and SARS-CoV-2 vaccine studies. S1 with sequence of the original Wuhan strain was used in the first three vaccinations. When evaluating the S1-specific IgG antibody responses, we found that this vaccine regimen induced a moderate level of humoral immune responses in serum and BAL fluid (FIG. 16A). Compared to the half-maximal binding titer of 25, 209 induced by IM-primed and -boosted alum-adjuvanted subunit vaccine (20), the peak median serum half-maximal binding titer was only 945 (FIG. 16A). Moreover, the vaccine-induced immunity also waned with time. After one year, the IgG responses in the vaccinated animals were comparable to those of the naïve controls (FIG. 16A).

The animals were then administered one intranasal booster with S1 from beta variant adjuvanted with CP15 in 1,2-dioleoyl-3-trimethylammonium-propane (DOTAP) nanoparticles. After the booster, significant anamnestic responses were elicited. The Log (half-maximal binding titer) of serum IgG titer reached 5.83 for the original Wuhan strain, and 5.08 for the beta variant, compared to the highest IgG titer of 2.77 logs at 2 weeks post the 3rd vaccination one year earlier (FIG. 16A). The booster also led to the induction of a substantial increase of binding IgG responses against both the original Wuhan strain and the beta variant S1 in BAL (FIG. 16A).

High titers of live virus neutralization antibody (Nab) responses against both WA1/2020 D614G SARS-CoV-2 (WA) strain and the beta variant were detected in the serum. The geometric mean titers (GMT) of Nab were 434 and 540 for ID50, and 60 and 145 for ID90 for the WA strain and the beta variant, respectively (FIG. 16B). Given the fact that the beta variant has been a difficult strain to neutralize, boosting with beta variant S1 might account for this improvement and indicates a benefit of switching antigens from the original WA strain to a variant. Boosting with the variant S1 still induced a strong anamnestic response against the original priming Wuhan S1.

IgA and dimeric IgA responses in bronchoalveolar lavage (BAL) and nasal swabs were also examined, as IgA, especially dimeric IgA, displays high binding avidity to pathogens, and thus is more potent at preventing mucosal pathogen infections. Right before the one-year booster, no S1 (original or beta variant)-specific IgA, or dimeric IgA responses were detected, and the antibody titers were comparable to the basal levels of naïve animals (FIG. 16C). Consistent with IgG and neutralization responses, the one-year booster enhanced IgA responses in nasal swab and BAL samples with similar antibody titers against S1 from the original strain and the beta variant (FIG. 16C). However, dimeric IgA responses against beta variant were not induced in BAL samples, whereas increased dimeric IgA responses were observed in BAL against the Wuhan strain and in nasal swabs against both strains (FIG. 16C).

These results showed that the one-year booster induced robust S1-specific antibody responses in serum and BAL, including potent neutralizing antibody responses in peripheral blood. Mucosal IgA responses were induced in nasal swabs and BAL that were comparable against both the original priming Wuhan strain and the beta variant, except dimeric IgA responses against beta variant in BAL.

Variant S1-Specific Cellular Responses were Induced after the One-Year Booster

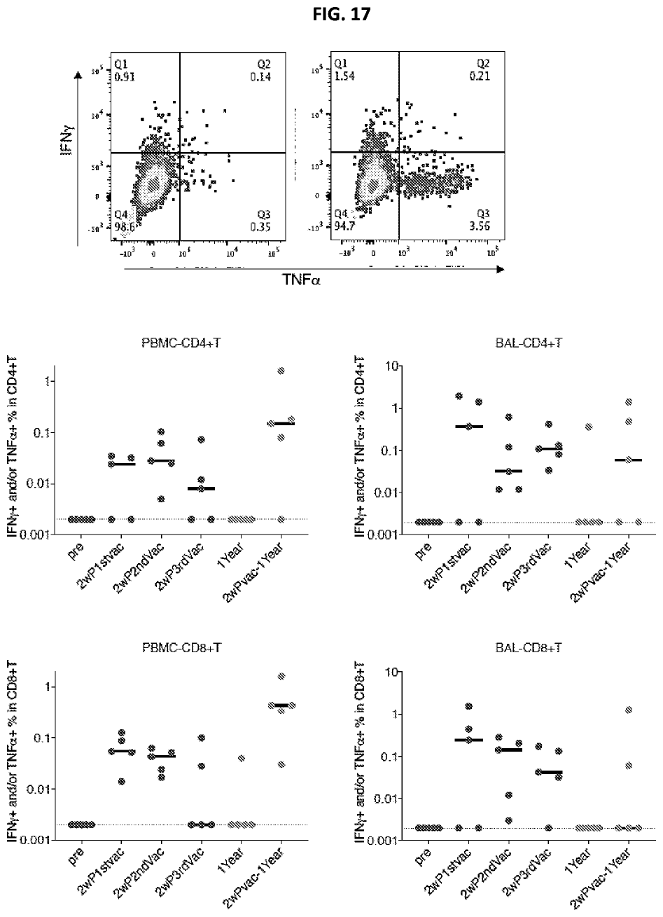

The vaccine-induced S1-specific T cell responses in PBMC and BAL samples of the vaccinated animals were evaluated by intracellular cytokine staining. S1-specific type 1 helper T cell responses (Th1) and CD8+ T cell that secrete tumor necrosis factor (TNF)-α, and/or interferon (IFN)-γ were induced after the first vaccination (FIG. 17). Though the responses were persistent in most of the vaccinated animals, no further enhancement of the responses was observed after the second and third vaccinations. A significant number of IL-2-producing cells was not observed. For CD8+ T cell responses, especially the responses in PBMC, a declining trend with each vaccination (less so in BAL) was observed. This raises the concern that extensive boosters in a short period of time might lead to the exhaustion of the SARS-CoV-2-specific T cell responses. In any case, the responses waned to under the detection limit in most of the animals after one year. After the administration of the one-year beta-variant booster, the S1-specific CD8+ T cell responses were successfully recalled in all 5 PBMC samples and CD4⁺ responses in 4/5 (FIG. 17). Even though the route of the one-year booster was intranasal, S1-specific CD4+ T cells were induced only in 3 BAL samples, and CD8⁺ T cells in only two.

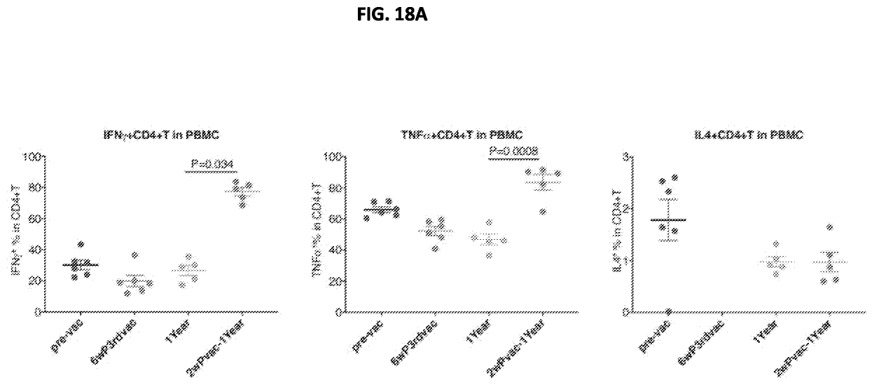

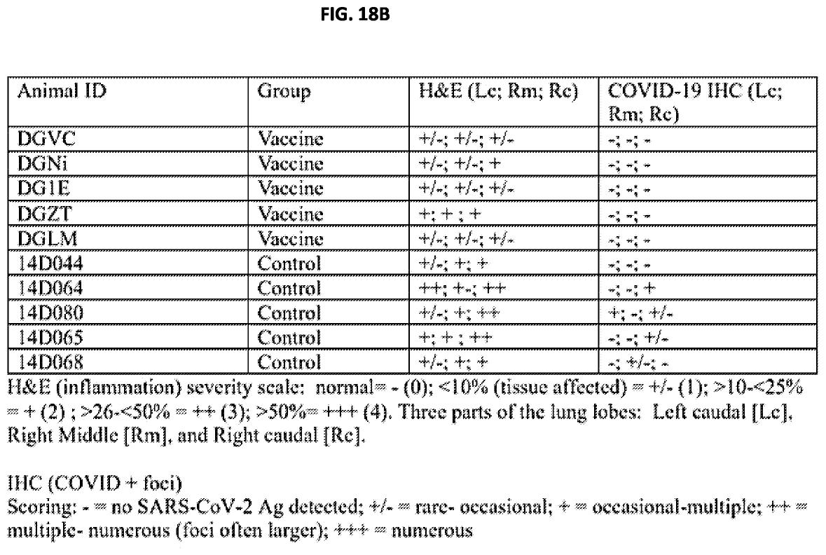

As the frequencies of antigen-specific T cell responses were low, the kinetics of total Th1 and Th2 subsets after stimulation with Phorbol 12-myristic 13-acetate (PMA) and ionomycin were assessed. There were no significant alterations after the first three vaccinations in the prior year (FIG. 18). However, the one-year boost resulted in sharp increase of Th1 responses in PBMC while the Th2 responses did not change (FIG. 18).

### Vaccinated Animals Demonstrated Significant Protection in BAL, and Almost Full Protection in Nasal Swabs Against SARS-CoV-2 Beta Variant Replication

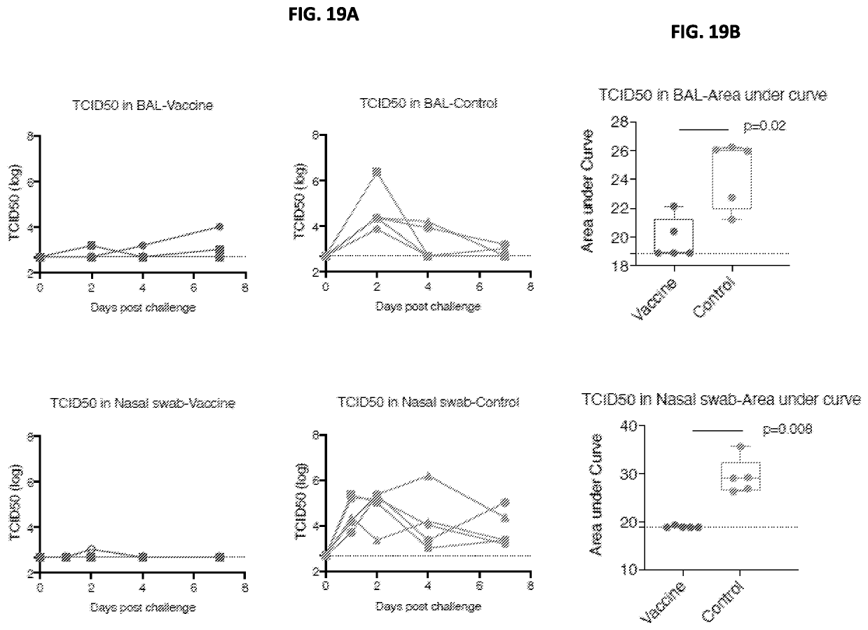

To test the protective efficacy against SARS-CoV-2 beta variant, 5 vaccinated and 5 naïve macaques were challenged with 1.0×10\hat{ }5 TCID50 SARS-CoV-2 beta variant (isolate beta variant B.1.351, in-house generated stock from BEI Resources, NR-54974) through intranasal (1 mL) and intratracheal (1 mL) routes 4 weeks after the last vaccination. Viral tissue culture infectious dose 50 titers (TCID50) were measured in the collected nasal swab and lung BAL samples. Replicating viruses were detected in both nasal swabs and BAL samples of all five naïve animals, indicating that the viral inoculation was successfully delivered and propagated in the upper and lower airways (FIG. 19A). The inoculation of SARS-CoV-2 beta variant led to prolonged detection of replicating virus in the nasal turbinate of the naïve animals. High levels of viral replication were present in all five naïve animals at day 7 post virus challenge. In contrast, the vaccinated animals demonstrated almost full protection in nasal swabs: only one animal showed a small blip at day 2 post viral challenge, while four other vaccinated animals were free of replicating virus during the 7-days post-challenge period (FIG. 19A). The vaccinated group showed significant reduction of viral replication in both nasal turbinate and lungs compared to naïve controls, based on the area under the curves over all time points (FIG. 19B).

Histopathology in the Lungs after Viral Infection Indicates Protection in the Lungs of Vaccinated Animals

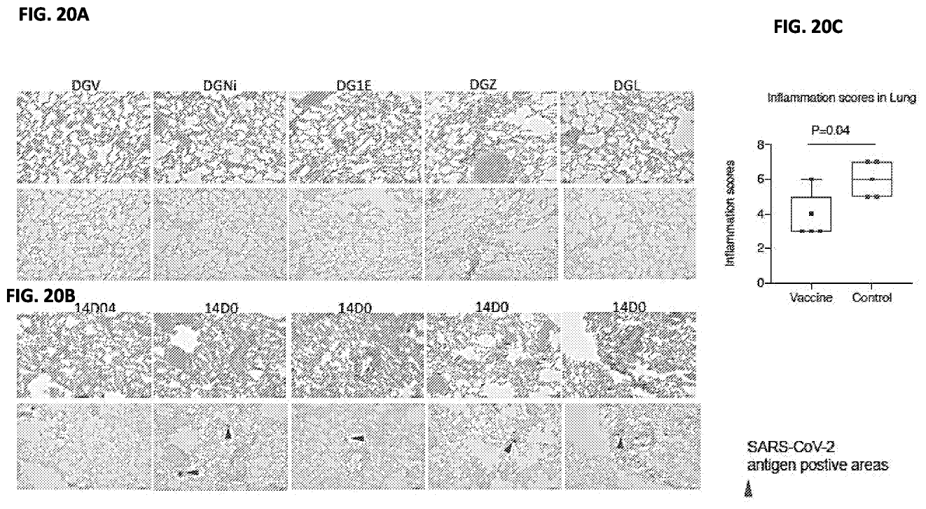

The mucosal vaccine is safe and the vaccinations were well tolerated. Throughout the whole course of this study, no adverse effects were observed in the vaccinated animals. When the animals were necropsied on day 7, sections of lung were evaluated immunohistochemically for SARS-CoV-2 virus antigen and histologically for the presence of SARS-CoV-2-associated inflammation. None of the 5 vaccinated animals demonstrated immunoreactivity to viral antigens, while virus antigens were detected in the lung sections of the 4 out of 5 animals in the control group (FIGS. 20A, 20B). Predominantly perivascular to interstitial inflammation was observed in the control group. An inflammation score was given to each animal blindly by a certified pathologist based on the evaluation of lung infiltration collected at the time of necropsy at day 7 post SARS-CoV-2 challenges (FIG. 18B). As beta variant led to persistent viral replication in the lungs of both naïve and vaccinated animals, the inflammation scores even in the vaccinated animals was not zero at day 7 post infection, which is consistent with the viral load data (FIG. 19A). However, the inflammation score was significantly more severe in the control group than in the vaccinated group (FIG. 20C).

### DISCUSSION

An additional booster vaccine is likely needed to curb the resurgence of SARS-CoV-2 cases. It is shown herein that the one-year beta variant mucosal booster given intranasally elicited high quality immune responses and mediated protections against subsequent SARS-CoV-2 beta variant viral challenge in rhesus macaques. The protection in the upper respiratory tract seemed to be better than that in the lower respiratory tract, which is different from most of the systemic vaccines. The nearly full protection against viral replication in the nasal cavity indicates its potential to prevent viral spread and transmission. During SARS-CoV-2 infection, the nasal mucosa is usually the first site of viral replication, so the local immunity induced by vaccination might be able to abort viral replication here before it disseminates systemically and may also prevent spread to other individuals. Indeed, high titers of mucosal IgA responses against both original and variant spike proteins were induced in the nasal mucosa, which might account for the efficient clearing of the virus in situ. These findings show the utility of a nasal mucosal vaccine as a booster rather than another systemic (IM) vaccine dose.

Waning immunity over time after vaccination/infection is contributing significantly to the resurgence of SARS-CoV-2 cases. Though the immune correlates of protection have not been fully established, neutralizing antibody (Nab) responses are believed to be one of the major protective mechanisms.

The emergence of SARS-CoV-2 variants of concern might partially account for the reported decreased vaccine effectiveness after 6 months. These variants either have high infectious potency or evade the immunity induced by SARS-CoV-2 infection or vaccination. The beta variant has the greatest immune evasive capacity among the widespread variants detected prior to omicron. We switched the S1 from original Wuhan strain to that of the beta variant, which led to successful elicitation of systemic and mucosal immune responses against both the original strain and the beta variant, and mediated protection against subsequent SARS-CoV-2 beta variant challenge. Incorporating S1 from the beta variant into the booster vaccine may account for the observed robust protection.

A dramatic increase in antibody titers after the one-year booster was observed (more than 3 log of increase compared to the highest titers one year before for serum IgG titers). This is consistent with what was found in a previous study, where the booster at 4 months induced much higher quality SARS-CoV-2 specific immune responses than the booster at 3 weeks did. This could be due to the DOTAP nanoparticles with beta S1 incorporated in the one-year booster rather than the PLGA nanoparticles with Wuhan S1 used before. Another possibility is that the longer interval between the booster and the previous vaccinations enhances the immune responses. Similar phenomena were reported in AstraZeneca (AZ) and inactivated vaccine trials, as well as in the standard hepatitis B viral vaccine regimen. In the AZ trial, a longer prime-boost interval (>12 weeks) led to higher vaccine efficacy compared to shorter interval (<6 weeks) (43). In an inactivated vaccine trial, 6 or more months between the second and third vaccinations also induced a remarkable increase in antibody levels compared to a 4-week interval.

The CP15 adjuvanted vaccine described herein was not very effective as a prime vaccine. It did not induce robust immune responses compared to an alum adjuvanted vaccine. One year after the first vaccination, no virus-specific humoral or cellular immunity was detected. Nevertheless, the one-year booster elicited high quality immune responses, and mediated protection against subsequent beta variant challenge, which indicates that the vaccinations in the prior year generated persistent SARS-CoV-2 specific immune memory. The specific immune memory may include antigen-specific long-lived B memory cells in bone memory and/or innate cell-mediated trained immunity. Though the humoral and cellular immune responses waned to undetectable levels after one year, the immune memory persisted, which facilitated the later recall responses, when boosted. Moreover, the data indicate that a weaker variant-modified booster vaccine might be sufficient to induce protective immunity in previously vaccinated hosts. These findings can guide prime-boosting regimens for COVID-19.

### Example 9

### Hamster Studies

Studies similar to those described above were performed in hamsters. Hamsters were chosen, as like humans, they get more severe disease in response to SARS-CoV-2 infection. In these studies, weight loss was used as an indicator of disease. Thus, hamsters serve as a good model for COVID-19 disease (while macaques are a model of SARS-CoV-2 infection). In addition, as more subjects could be tested, males and females were used.

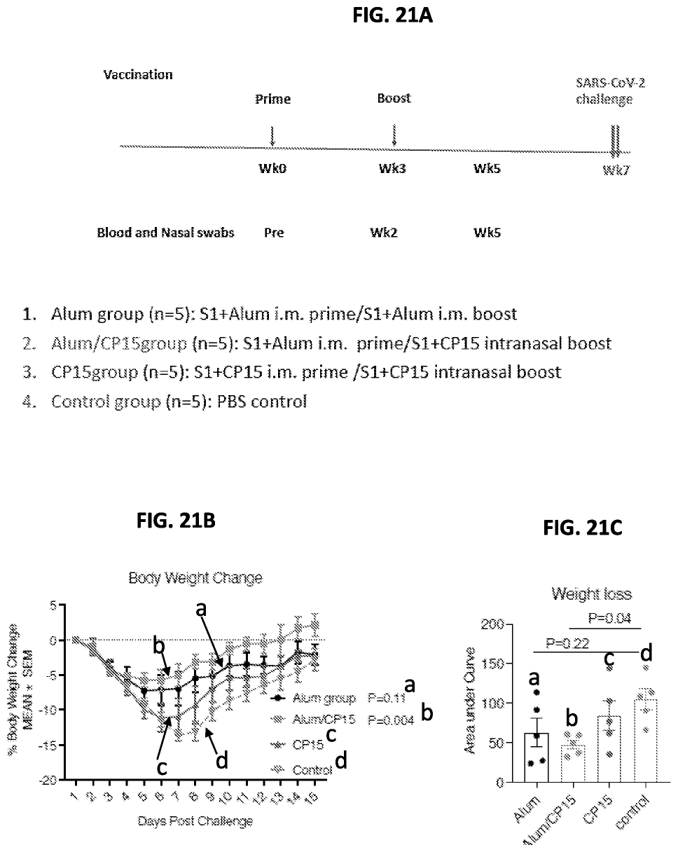

A schematic of the methods used are shown in FIG. 21A. Hamsters were either primed and boosted with S1 protein adjuvanted with alum IM (group 1), primed with S1 protein adjuvanted with alum IM and then boosted with S1/IL15/CpG/poly:IC (referred to as S1+CP15) IN (group 2), primed with S1+CP15 IM then boosted with 1+CP15 IN (group 3), or administered PBS as a negative control (group 4). The hamsters were then subsequently challenged with SARS-CoV-2 WA strain. Blood, oral, and nasal swabs were obtained (and viral load determined), and body weight monitored. As shown in FIGS. 21B-21C, hamsters in Group 2 lost less weight than the other groups, indicating that primed with S1 protein adjuvanted with alum IM and then boosting IN with S1+CP15 provides significant protection against disease associated with SARS-Cov-2 WA strain, while Group 1 (S1+alum) showed some protection. Hamsters in Groups 3 and 4 did not receive protection against weight loss.

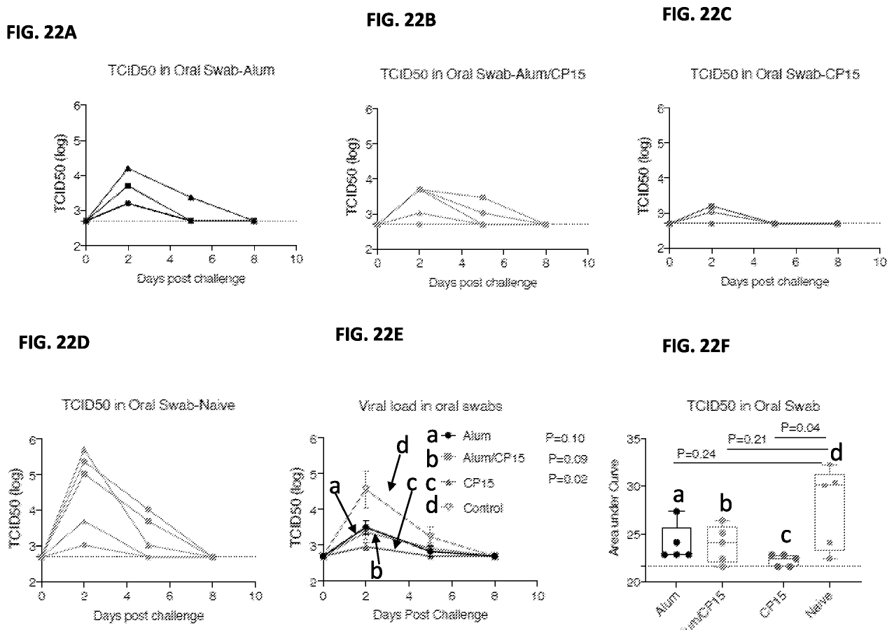

After challenge with SARS-CoV-2 WA strain, oral swabs from each hamster were collected at different time points and viral loads were measured in TCID50. As shown in FIGS. 22A-22F, hamsters IM primed and IN boosted with S1+CP15 (group 3), showed significant oral swab viral load reduction after challenge with SARS-Cov-2 WA strain (FIG. 22C), while S1+alum and Alum/CP15 vaccinated groups showed trends of protection in Syrian golden hamsters (FIGS. 22A, 22B). Summaries are provided in FIGS. 22E, 22F.

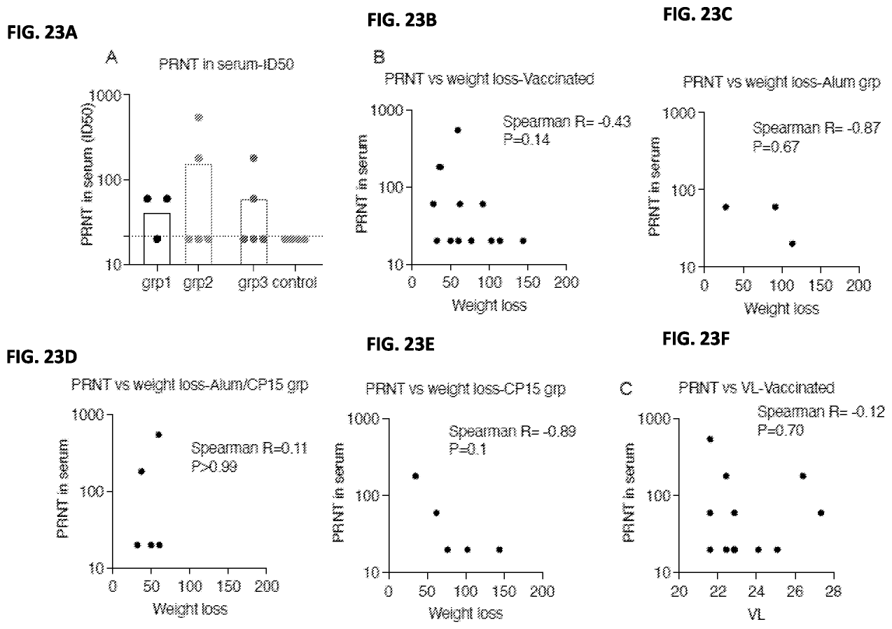

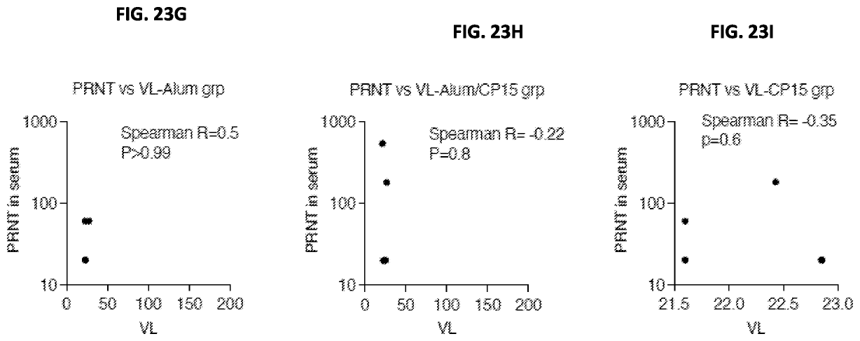

PRNT titers against SARS-CoV-2 WA strain were determined in the serum of vaccinated and naïve animals. As shown in FIGS. 23A-23I, there was no correction between the presence of neutralizing antibodies and a protective effect.

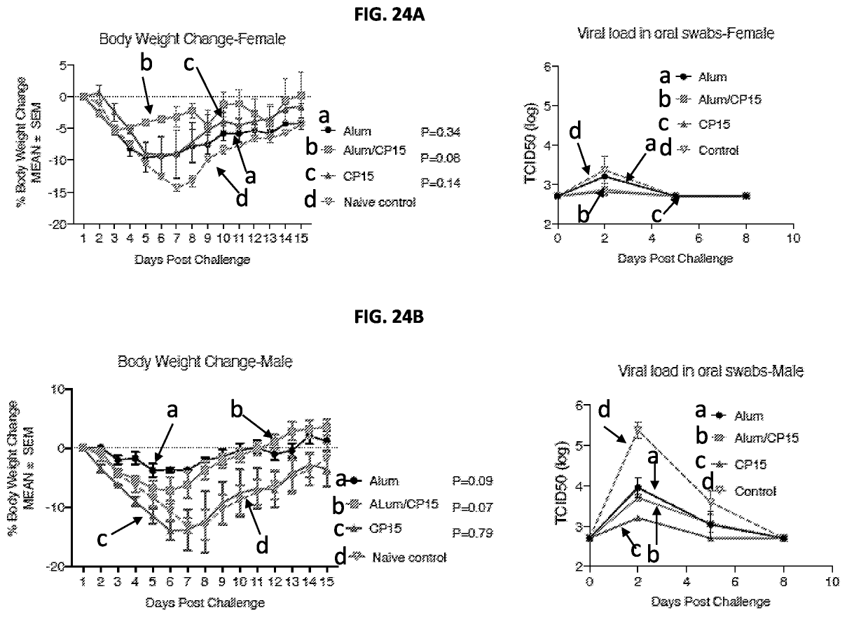

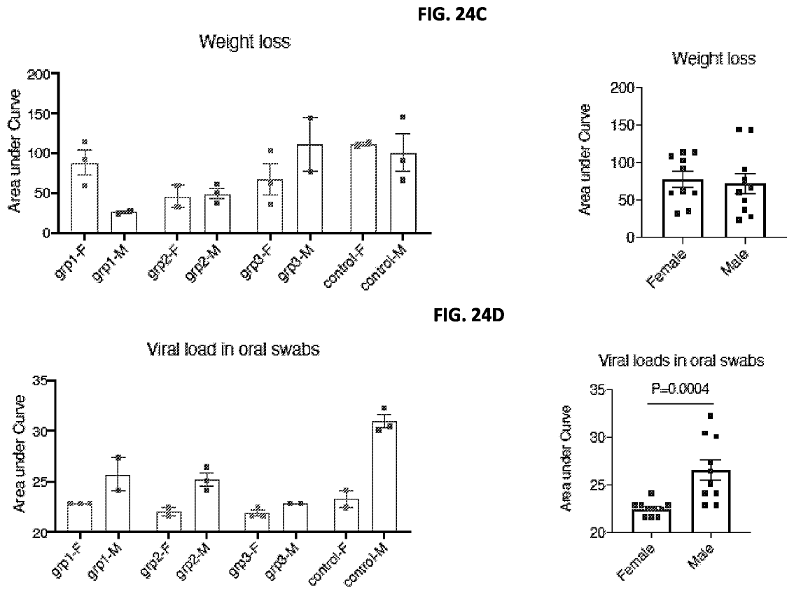

Gender differences in viral load and weight loss were observed. Females generated a better response than males. As shown in FIGS. 24A-24C, females lost less weight than males, indicating they had less disease and a more protective response. As shown in FIGS. 24A-24B, 24D, females also had a reduced viral load detected in oral swabs.

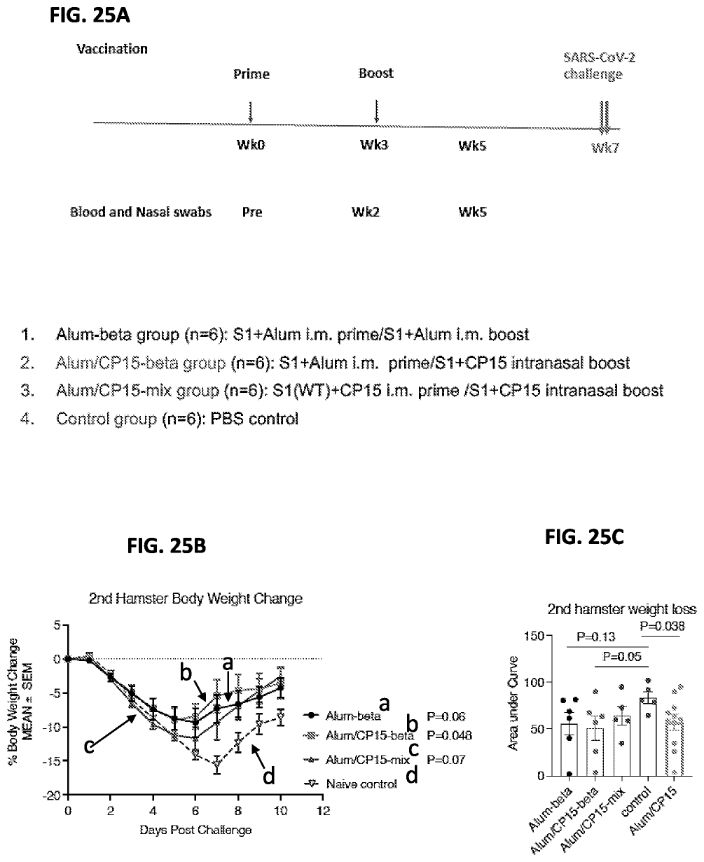

The ability of the four different groups to mount a protective immune response to challenge with SARS-CoV-2 beta variant was tested as shown FIG. 25A. Hamsters were either primed and boosted with S1 protein adjuvanted with alum IM (group 1), primed with S1 protein adjuvanted with alum IM and then boosted with S1/IL15/CpG/poly:IC (simply referred to as S1+CP15) IN (group 2), primed with S1+CP15 IM then boosted with S1+CP15 IN (group 3), or administered PBS as a negative control. The hamsters were then subsequently challenged with SARS-CoV-2 beta variant. Blood and nasal swabs were obtained (and viral load determined), and body weight monitored. As shown in FIGS. 25B-25C, hamsters in Group 2 lost less weight than the other groups, indicating that primed with S1 protein adjuvanted with alum IM and then boosting IN with S1+CP15 provides significant protection against disease associated with SARS-Cov-2 beta variant, while Group 1 (S1+alum) showed a trend of protection.

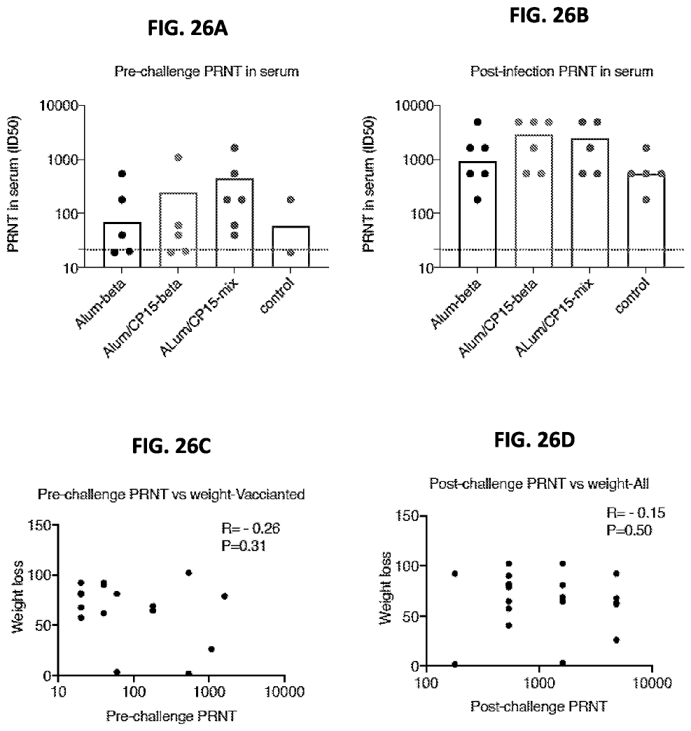

As shown in FIGS. 26A-26D, neither vaccine-induced nor virus-induced Nab titers correlated with protection against weight loss after challenged with SARS-CoV-2 beta variant in Syrian golden hamsters.

In summary, using a hamster model, it was demonstrated that both mucosal and systemic vaccines provided protections against SARS-CoV-2 Washington strain and beta variant infections (FIGS. 21, 22, and 25). The mucosal vaccine meditated better protection against weight loss compared to the systemic vaccine (FIGS. 21, and 25). Furthermore, there was a gender difference for the mucosal vaccine: the females achieved better protection by reducing the oral viral loads more than the males (FIG. 24D). In addition, the neutralizing antibodies were not associated with protection for the mucosal vaccine (FIG. 23).

### Example 10

### Female Mice Boosted IN have More Robust Lung S1-Specific Responses

Three groups of mice (n=10/group, half male and half female) were vaccinated as in Example 1. The same reagents (CP15) were used for mouse and hamster studies (Example 9). The mice were primed intramuscularly with S1+alum, and then followed by boost with intranasally administrated S1+CP15 at week 2. At week 4, the mice were scarified, and lungs were processed to get the single cell suspension. After stimulation these cells with S1 for 18 hrs, the frequencies of IFNγ, TFNα and IL2-producing CD4+ and CD8+ T cells were stained and measured.

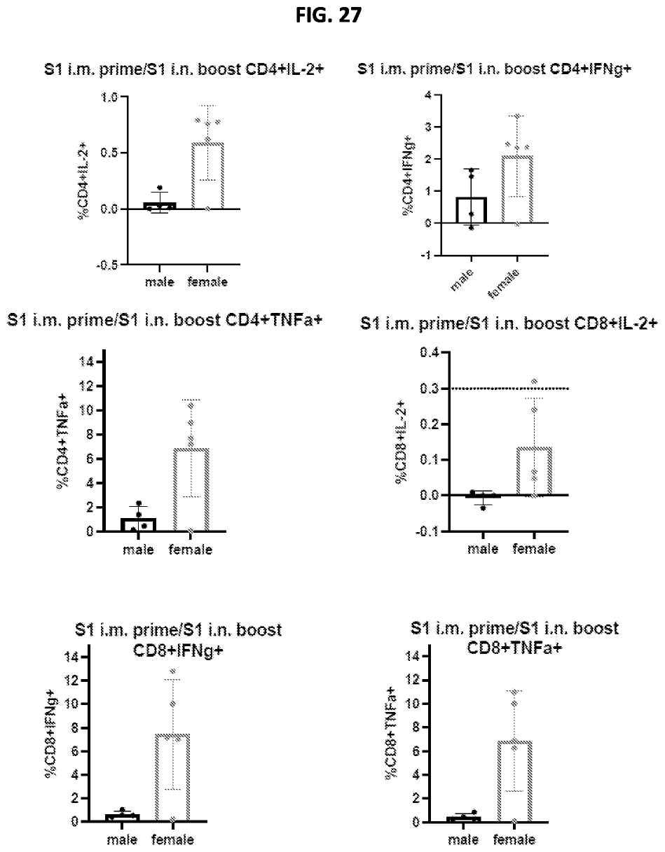

As shown in FIG. 27, only group 2, which were primed intramuscularly with S1+alum and boosted intranasally with S1+CP15, had detectable SARS-CoV-2-specific T cell responses in the lungs.

In view of the many possible embodiments to which the principles of the disclosed invention may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

1. An immunogenic composition, comprising
(a) a SARS-CoV-2 protein comprising
a SARS-COV-2 Spike 1 (S1) protein comprising at least 80%, least 85%, at least 90%, at least 95%, at least 96%, at least 97%, at least 98%, at least 99%, or 100% sequence identity to SEQ ID NO: 2, 3, 4, 5, 6, 12 or 15;
a SARS-COV-2 S2 protein comprising at least 80%, least 85%, at least 90%, at least 95%, at least 96%, at least 97%, at least 98%, at least 99%, or 100% sequence identity to SEQ ID NO: 9, 13 or 16; or
a SARS-COV-2 Spike (S) protein comprising at least 80%, least 85%, at least 90%, at least 95%, at least 96%, at least 97%, at least 98%, at least 99%, or 100% sequence identity to SEQ ID NO: 1, 11 or 14, or to amino acids 16-1208 of SEQ ID NO: 1, amino acids 16-1205 of SEQ ID NO: 11, or amino acids 16-1206 of SEQ ID NO: 14; and
(b) CpG oligodeoxynucleotide, Poly I:C and IL-15; or
CpG oligodeoxynucleotide, Poly I:C, IL-15, or combinations thereof.

2. The immunogenic composition of claim 1, wherein the immunogenic composition comprises CpG oligodeoxynucleotide, Poly I:C and IL-15.

3. (canceled)

4. The immunogenic composition of claim 1, further comprising a pharmaceutically acceptable carrier.

5. A nanoparticle, comprising
(a) a SARS-CoV-2 Spike (S) protein comprising
a SARS-COV-2 S1 protein comprising at least 80%, least 85%, at least 90%, at least 95%, at least 96%, at least 97%, at least 98%, at least 99%, or 100% sequence identity to SEQ ID NO: 2, 3, 4, 5, 6, 12 or 15;
a SARS-COV-2 S2 protein comprising at least 80%, least 85%, at least 90%, at least 95%, at least 96%, at least 97%, at least 98%, at least 99%, or 100% sequence identity to SEQ ID NO: 9, 13 or 16; or
a SARS-COV-2 S protein comprising at least 80%, least 85%, at least 90%, at least 95%, at least 96%, at least 97%, at least 98%, at least 99%, or 100% sequence identity to SEQ ID NO: 1, 11 or 14, or to amino acids 16-1208 of SEQ ID NO: 1, amino acids 16-1205 of SEQ ID NO: 11, or amino acids 16-1206 of SEQ ID NO: 14; and
(b) CpG oligodeoxynucleotide, Poly I:C and IL-15; or
CpG oligodeoxynucleotide, Poly I:C, IL-15, or combinations thereof.

6.-7. (canceled)

8. The nanoparticle of claim 5, wherein the nanoparticle comprises poly(d,l-lactide-co-glycolide) (PLGA) or 1,2-dioleoyl-sn-glycero-3-phosphoethanolamine (DOTAP).

9. An immunogenic composition comprising the nanoparticle of claim 5 and a pharmaceutically acceptable carrier.

10. A glass vial, plastic vial, or syringe, comprising the immunogenic composition of claim 1.

11. A method of eliciting an immune response against SARS-CoV-2 in a subject, comprising:
administering to the subject an effective amount of a prime dose of the immunogenic composition comprising a SARS-CoV-2 S protein, S1 protein, or S2 protein and an adjuvant; and
subsequently administering to the subject an effective amount of one or more booster doses of the immunogenic composition of claim 9;
thereby eliciting the immune response.

12. A method of eliciting an immune response against SARS-CoV-2 in a subject, comprising:
administering to the subject an effective amount of a prime dose of a SARS-CoV-2 vaccine; and
subsequently administering to the subject an effective amount of one or more booster doses of the immunogenic composition of claim 9;
thereby eliciting the immune response.

13. The method of claim 11, wherein a first booster dose is administered at least 2 weeks, at least 3 weeks, at least 4 weeks, at least 5 weeks, at least 6 weeks, at least 7 weeks, at least 8 weeks, at least 10 weeks, at least 12 weeks, at least 16 weeks, at least 6 months, at least 9 months, at least 12 months, at least 18 months, at least 24 months, or at least 36 months after administering the primary dose.

14. The method of claim 11, wherein the one or more booster doses comprises 2, 3, 4, 5, 6, 7, 8, 9 or 10 booster doses.

15. The method of claim 11, wherein the immune response:
inhibits or prevents SARS-CoV-2 infection in the subject;
inhibits or prevents severe COVID19 disease in the subject;
reduces the risk of transmission of SARS-CoV-2 to other subjects;
increases production of dimeric IgA specific for SARS-CoV-2 S1 and IFNα;
provides 100% protection against subgenomic viral RNA from SARS-CoV-2 viral challenges in the subject, for example in the upper and lower respiratory tracts;
induces a neutralizing antibody titer of at least 100, at least 200, at least 300, at least 350;
or combinations thereof.

16. (canceled)

17. The method of claim 1, wherein administering the primary dose comprises intramuscular administration and wherein administering the one or more booster doses comprises intranasal administration.

18. The method of claim 1, wherein generating the immune response inhibits replication of the SARS-CoV-2 in the subject.

19. The method of claim 12, wherein the SARS-CoV-2 vaccine is:
an mRNA vaccine, such as Pfizer-BioNTech's BNT162b2 vaccine, or Moderna's mRNA-1273 vaccine;
a chimpanzee adenovirus-vectored vaccine, such as AstraZeneca's ChAdOx1 nCoV-19 vaccine (AZC1222),
a protein vaccine, such as Novavax's NVX-CoV2373 vaccine;
an adenovirus serotype 26 vectored vaccine, such as Johnson & Johnson's JNJ-78436735 vaccine; or
a DNA vaccine, such as Inovio's INO-4800 vaccine.

20.-21. (canceled)

22. A method of eliciting an immune response against SARS-CoV-2 in a subject, comprising:
administering to the subject an effective amount of a primary dose an immunogenic composition comprising the SARS-CoV-2 S1 protein of SEQ ID NO: 2, and alum; and
subsequently administering to the subject an effective amount of one or more booster doses of nanoparticles comprising the SARS-CoV-2 S1 protein of SEQ ID NO: 2, CpG oligodeoxynucleotide, Poly I:C, and IL-15.

23. The method of claim 22, wherein nanoparticles in a first booster dose comprise PLGA or DOTAP.

24.-26. (canceled)

27. The method of claim 11, further comprising administering to the subject a COVID-19 treatment, such as remdesivir, galidesivir, lenzilumab, molnupiravir, hydroxychloroquine, dexamethasone, arbidol, favipiravir, baricitinib, lopinavir/ritonavir, zinc ions, and interferon beta-1b.

28. The immunogenic composition of claim 1, wherein the SARS-CoV-2 S protein, S1 protein, or S2 protein is from SARS-CoV-2 variant alpha; beta; delta; gamma; epsilon; eta (B.1.525); iota (B.1.526); kappa (B.1.617.1); 1.617.3; mu (B.1.621, B.1.621.1), zeta (P.2), or omicron (B.1.1.529).


SEQUENCE LISTING (16 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 670 aa, Severe acute respiratory syndrome coronavirus 2
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 3 - protein, 668 aa, Severe acute respiratory syndrome coronavirus 2 B.1.1.7
VNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSHRRAR

SEQ ID NO: 4 - protein, 670 aa, Severe acute respiratory syndrome coronavirus 2 B.1.351
VNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAR

SEQ ID NO: 5 - protein, 670 aa, Severe acute respiratory syndrome coronavirus 2 P.1
VNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRAR

SEQ ID NO: 6 - protein, 670 aa, Artificial Sequence
  SARS-CoV-2 S1 with  E484K and N501Y substitutions
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 7 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - DNA, 3822 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 9 - protein, 522 aa, Severe acute respiratory syndrome coronavirus 2
VASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 10 - DNA, 24 bp, Artificial Sequence
  CpG sequence
tcgtcgttttgtcgttttgtcgtt

SEQ ID NO: 11 - protein, 1285 aa, Severe acute respiratory syndrome coronavirus 2 B.1.1.529
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 12 - protein, 667 aa, Severe acute respiratory syndrome coronavirus 2 B.1.1.529
VNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSAS

SEQ ID NO: 13 - protein, 522 aa, Severe acute respiratory syndrome coronavirus 2 B.1.1.529
VASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 14 - protein, 1271 aa, Severe acute respiratory syndrome coronavirus 2 B.1.617.2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGFCSCGSCCKFDEDDSEPVLKGVKLHYT

SEQ ID NO: 15 - protein, 668 aa, Severe acute respiratory syndrome coronavirus 2 B.1.617.2
VNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRAR

SEQ ID NO: 16 - protein, 522 aa, Severe acute respiratory syndrome coronavirus 2 B.1.617.2
VASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ